(19) United States
(12) Patent Application Publication
(10) Pub. No.: US 2025/0197478 A1
(43) Pub. Date: Jun. 19, 2025

(54) COMPOSITIONS FOR INCREASING HALF-LIFE OF A THERAPEUTIC AGENT IN LIVESTOCK ANIMALS AND METHODS OF USE

(71) Applicant: Invetx, Inc., Boston, MA (US)

(72) Inventors: William BRONDYK, Brewster, MA (US); Juergen HORN, Marblehead, MA (US)

(21) Appl. No.: 18/816,282

(22) Filed: Aug. 27, 2024

Related U.S. Application Data
(63) Continuation of application No. 17/540,044, filed on Dec. 1, 2021, now Pat. No. 12,103,961.
(60) Provisional application No. 63/122,365, filed on Dec. 7, 2020.

(51) Int. Cl. C07K 16/00

(52) U.S. Cl. CPC C07K 16/00; C07K 2317/72; C07K 2317/92; C07K 2317/94

(57) ABSTRACT

Provided are compositions for increasing the half-life of a polypeptide or polypeptides in a livestock animal and methods of their use. The compositions involve variant IgG Fc regions.

### FIELD

This disclosure relates generally to polypeptides (e.g., fusion polypeptides such as polypeptide-Fc region fusions; or binding molecules such as antibodies or ligand-binding portions of receptor-Fc fusions) that have increased half-life in livestock animals compared to their wild type counterparts.

### SEQUENCE LISTING

The present application contains a Sequence Listing which has been submitted in XML format and is hereby incorporated by reference in its entirety. Said XML copy, created on Mar. 10, 2025, is named 51682-005003_Sequence_Listing_3_10_25 and is 39,224 bytes in size.

### BACKGROUND

The Fc region of antibodies plays a number of functional roles, including, but not limited to, protecting the antibody from degradation through the lysosomal pathway and mediating antibody effector functions. With the increasing use of antibodies as therapeutic agents in livestock animals, there has been an enhanced focus on not just selecting an optimal Fab, but also combining it with an appropriate Fc for desired half-life and effector functions.

There is little guidance in the art relating to increasing half-life of polypeptide therapeutics (e.g., antibodies) for use in livestock animals. This disclosure remedies that failing by providing Fc region variants that improve the serum persistence of polypeptides (e.g., antibodies) in livestock animals.

### SUMMARY

Provided herein are Fc (e.g., IgG Fc region variant) of livestock animals or FcRn binding fragments thereof that are useful in therapeutic polypeptides. This disclosure features polypeptides that have increased binding to FcRn of livestock animals than control polypeptides (e.g., the wild type counterpart IgG Fc regions). In some instances, these polypeptides have increased binding to FcRn livestock animals when compared to control polypeptides at pH 5.5, pH 6.0, and/or pH 6.5. In some instances, these polypeptides can, e.g., bind to FcRn of livestock animals at a higher level at acidic pH (e.g., pH 5.5, pH 6.0, or pH 6.5) than at a neutral pH (e.g., pH 7.0, 7.1, 7.2, 7.3, 7.4, or 7.5). In some instances, these polypeptides bind to FcRn of livestock animals at a higher level at pH 5.5 and/or 6.0 than at pH 7.4. This disclosure relates, in part, to polypeptides that have increased half-life in livestock animals than their wild type counterparts. For example, provided are binding molecules (e.g., antibodies or ligand-binding portions of receptors) with increased half-life relative to versions of these binding molecules not attached to the Fc regions or FcRn binding regions thereof disclosed herein. Also provided are enzyme-Fc region fusions, ligand-Fc region fusions, nanobody-Fc fusions, and peptide-Fc region fusions, wherein the fusions have increased half-life compared with their wild type counterparts. The Fc regions, in addition to having a substitution or substitutions (relative to the wild type Fc region of a livestock animal of the same species) that increase half-life, may also include other substitutions that, e.g., increase effector function, decrease effector function, increase binding to Protein A, and/or decrease heterogeneity of the polypeptide (e.g., by removing one or more post-translational modifications in the Fc region). The Fc region sequences of livestock animals can be from any antibody of the livestock animal. In some instances, the Fc region sequences of the livestock animal are from an IgG, subclasses of which are described elsewhere herein.

The disclosure features a recombinant protein comprising (1) a binding domain, or a fragment thereof, that specifically binds to a ligand, or an epitope of a protein, wherein the binding domain is attached to (2) a domain comprising an Fc region (CH2+CH3 region) of a livestock animal, or an FcRn binding region thereof, as disclosed herein. In some instances, the binding domain comprises (i) the six complementarity determining regions (CDRs), for example, of a livestock animal or human/humanized antibody; (ii) a nanobody; (iii) a soluble receptor-binding domain that binds a ligand, or a ligand-binding fragment thereof, and (iv) an extracellular domain of a livestock animal receptor protein.

The disclosure also provides a composition comprising: (1) a first polypeptide comprising a first Fc region (e.g., a CH2 domain, a CH3 domain, a CH2+CH3 domain) comprising a livestock animal IgG Fc region variant described herein; and (2) a second polypeptide comprising a second Fc region comprising a livestock animal IgG Fc region variant described herein. The first and second polypeptide can be associated through the first and second Fc regions. In some instances, the amino acid sequences of the first and second Fc regions are the same. In other instances, the amino acid sequences of the first and second Fc regions are different (e.g., by 1, 2, 3, 4, 5, 6, 7, 8, 9, 10, 11, 12, 13, 14, 15, 16, 17, 18, 19, 20, 21, 22, 23, 24, or 25 amino acids).

In some embodiments, the Fc region variant is an Fc variant of a porcine IgG selected from the group consisting of IgG1a, IgG1b, IgG2a, IgG2b, IgG3, IgG4a, IgG4b, IgG5a, IgG5b, IgG6a, and IgG6b.

In some embodiments, the Fc region variant is an Fc variant of a bovine IgG selected from the group consisting of IgG1, IgG2, and IgG3.

In some embodiments, the Fc region variant is an Fc variant of an equine IgG selected from the group consisting of IgG1, IgG2, IgG3, IgG4, IgG5, IgG6, and IgG7.

In some embodiments, the Fc region variant is an Fc variant of an ovine IgG selected from the group consisting of IgG1, IgG2, and IgG3.

In some embodiments, the Fc region variant is an Fc variant of a caprine IgG selected from the group consisting of IgG1, IgG2, and IgG3.

Also disclosed is a fusion molecule comprising a livestock animal IgG Fc region variant disclosed herein and a polypeptide. In some instances, the livestock animal IgG Fc region variant is covalently attached to the polypeptide (e.g., through a hinge region or a linker). In some instances, the polypeptide is a ligand binding domain of a livestock animal receptor protein, an extracellular domain of a livestock animal receptor protein, or an antigen-binding domain. In some instances, the polypeptide is selected from the ligand binding domain or extracellular domain of an IL-13Rα1, or IL-13Rα2, a livestock animal erythropoietin (EPO), a livestock animal CTLA4, a livestock animal LFA3, a livestock animal VEGFR1/VEGFR3, a livestock animal IL-1R, a livestock animal GLP-1 receptor agonist, and a livestock animal thrombopoietin binding peptide. In some instances, the polypeptide is an scFv, a nanobody, or single domain antibody. In some instances, the IgG Fc region variant is a variant of a livestock animal IgG1a antibody Fc region. In some instances, the IgG Fc region variant is a variant of a livestock animal IgG1b antibody Fc region. In some instances, the IgG Fc region variant is a variant of a livestock animal IgG2 antibody Fc region.

In some aspects, the disclosure provides a polypeptide comprising an IgG Fc region variant of a livestock animal, or an FcRn-binding region thereof, wherein the polypeptide comprises an amino acid substitution at at least one position selected from the group consisting of:
(i) a position that corresponds to amino acid position 250 of a wild type IgG of the livestock animal, wherein the amino acid substitution is selected from the group consisting of S250Q, S250E, T250Q, T250E, I250Q, I250E, A250Q, A250E, V250Q, and V250E;
(ii) a position that corresponds to amino acid position 252 of a wild type IgG of the livestock animal, wherein the amino acid substitution is selected from the group consisting of M252Y, M252W, M252F, T252Y, T252W, T252F, K252Y, K252W, and K252F;
(iii) a position that corresponds to amino acid position 254 of a wild type IgG of the livestock animal, wherein the amino acid substitution is selected from the group consisting of S254T, S254R, S254K, S254H, T254R, T254K, and T254H;
(iv) a position that corresponds to amino acid position 256 of a wild type IgG of the livestock animal, wherein the amino acid substitution is selected from the group consisting of T256A, T256D, T256E, M256A, M256D, M256E, K256A, K256D, and K256E;
(v) a position that corresponds to amino acid position 286 of a wild type IgG of the livestock animal, wherein the amino acid substitution is selected from the group consisting of S286Y, S286F, S286W, S286L, S286D, S286E, T286Y, T286F, T286W, T286L, T286D, and T286E;
(vi) a position that corresponds to amino acid position 309 of a wild type IgG of the livestock animal, wherein the amino acid substitution is selected from the group consisting of Q309V, Q309D, and Q309E;
(vii) a position that corresponds to amino acid position 311 of a wild type IgG of the livestock animal, wherein the amino acid substitution is selected from the group consisting of Q311V, Q311K, Q311R, Q311L, Q311H, E311V, E311K, E311R, E311L, E311H, K311V, K311R, K311L, K311H, D311V, D311K, D311R, D311L, and D311H;
(viii) a position that corresponds to amino acid position 426 of a wild type IgG of the livestock animal, wherein the amino acid substitution is selected from the group consisting of A426Y, A426F, A426H, V426Y, V426F, V426H, G426Y, G426F, and G426H;
(ix) a position that corresponds to amino acid position 428 of a wild type IgG of the livestock animal, wherein the amino acid substitution is selected from the group consisting of M428L, M428Y, M428F, H428L, H428Y, and H428F;
(x) a position that corresponds to amino acid position 434 of a wild type IgG of the livestock animal, wherein the amino acid substitution is selected from the group consisting of N434A, N434H, N434F, N434S, N434W, N434Y, H434A, H434F, H434S, H434W, and H434Y; and
(x) a position that corresponds to amino acid position 436 of a wild type IgG of the livestock animal, wherein the amino acid substitution is selected from the group consisting of Y436H, T436H, F436H, and V436H;
wherein the amino acid positions are based on EU numbering, and wherein the polypeptide has increased binding affinity to an FcRn of the livestock animal when compared to an Fc domain of the wild type IgG.

In some embodiments, the livestock animal is a porcine.

In some embodiments, the polypeptide comprises an amino acid substitution at at least one position selected from the group consisting of:
(i) a position that corresponds to amino acid position 250 of a wild type IgG of the livestock animal, wherein the amino acid substitution is selected from the group consisting of T250Q, T250E, I250Q, and I250E;
(ii) a position that corresponds to amino acid position 252 of a wild type IgG of the livestock animal, wherein the amino acid substitution is selected from the group consisting of M252Y, M252W, and M252F;
(iii) a position that corresponds to amino acid position 254 of a wild type IgG of the livestock animal, wherein the amino acid substitution is selected from the group consisting of S254T, S254R, S254K, and S254H;
(iv) a position that corresponds to amino acid position 256 of a wild type IgG of the livestock animal, wherein the amino acid substitution is selected from the group consisting of T256A, T256D, and T256E;
(v) a position that corresponds to amino acid position 286 of a wild type IgG of the livestock animal, wherein the amino acid substitution is selected from the group consisting of T286Y, T286F, T286W, T286L, T286D, and T286E;
(vi) a position that corresponds to amino acid position 309 of a wild type IgG of the livestock animal, wherein the amino acid substitution is selected from the group consisting of Q309V, Q309D, and Q309E;
(vii) a position that corresponds to amino acid position 311 of a wild type IgG of the livestock animal, wherein the amino acid substitution is selected from the group consisting of Q311V, Q311K, Q311R, Q311L, Q311H, E311V, E311K, E311R, E311L, and E311H;
(viii) a position that corresponds to amino acid position 426 of a wild type IgG of the livestock animal, wherein the amino acid substitution is selected from the group consisting of A426Y, A426F, A426H, V426Y, V426F, and V426H;
(ix) a position that corresponds to amino acid position 428 of a wild type IgG of the livestock animal, wherein the amino acid substitution is selected from the group consisting of M428L, M428Y, M428F, H428L, H428Y, and H428F;
(x) a position that corresponds to amino acid position 434 of a wild type IgG of the livestock animal, wherein the amino acid substitution is selected from the group consisting of N434A, N434H, N434F, N434S, N434W, N434Y, H434A, H434F, H434S, H434W, and H434Y; and
(xi) a position that corresponds to amino acid position 436 of a wild type IgG of the livestock animal, wherein the amino acid substitution is selected from the group consisting of Y436H and T436H.

In some embodiments, the polypeptide comprises an amino acid substitution at at least one position selected from the group consisting of:
(i) a position that corresponds to amino acid position 252 of a wild type IgG of the livestock animal, wherein the amino acid substitution is M252Y;
(ii) a position that corresponds to amino acid position 286 of a wild type IgG of the livestock animal, wherein the amino acid substitution is T286E;
(iii) a position that corresponds to amino acid position 309 of a wild type IgG of the livestock animal, wherein the amino acid substitution is Q309D or Q309V;
(iv) a position that corresponds to amino acid position 311 of a wild type IgG of the livestock animal, wherein the amino acid substitution is Q311V;
(v) a position that corresponds to amino acid position 426 of a wild type IgG of the livestock animal, wherein the amino acid substitution is A426Y;
(vi) a position that corresponds to amino acid position 428 of a wild type IgG of the livestock animal, wherein the amino acid substitution is M428Y; and
(vii) a position that corresponds to amino acid position 434 of a wild type IgG of the livestock animal, wherein the amino acid substitution is selected from the group consisting of N434Y.

In some embodiments, the wild type IgG Fc is a porcine IgG Fc comprising an amino acid sequence selected from the group consisting of SEQ ID NOs: 1 to 11.

In some embodiments, the livestock animal is a bovine.

In some embodiments, the polypeptide comprises an amino acid substitution at at least one position selected from the group consisting of:
(i) a position that corresponds to amino acid position 250 of a wild type IgG of the livestock animal, wherein the amino acid substitution is selected from the group consisting of T250Q and T250E;
(ii) a position that corresponds to amino acid position 252 of a wild type IgG of the livestock animal, wherein the amino acid substitution is selected from the group consisting of M252Y, M252W, M252F, T252Y, T252W, and T252F;
(iii) a position that corresponds to amino acid position 254 of a wild type IgG of the livestock animal, wherein the amino acid substitution is selected from the group consisting of S254T, S254R, S254K, S254H, T254R, T254K, and T254H;
(iv) a position that corresponds to amino acid position 256 of a wild type IgG of the livestock animal, wherein the amino acid substitution is selected from the group consisting of T256A, T256D, and T256E;
(v) a position that corresponds to amino acid position 286 of a wild type IgG of the livestock animal, wherein the amino acid substitution is selected from the group consisting of T286Y, T286F, T286W, T286L, T286D, and T286E;
(vi) a position that corresponds to amino acid position 309 of a wild type IgG of the livestock animal, wherein the amino acid substitution is selected from the group consisting of Q309V, Q309D, and Q309E;
(vii) a position that corresponds to amino acid position 311 of a wild type IgG of the livestock animal, wherein the amino acid substitution is selected from the group consisting of Q311V, Q311K, Q311R, Q311L, and Q311H;
(viii) a position that corresponds to amino acid position 426 of a wild type IgG of the livestock animal, wherein the amino acid substitution is selected from the group consisting of A426Y, A426F, A426H, V426Y, V426F, and V426H;
(ix) a position that corresponds to amino acid position 434 of a wild type IgG of the livestock animal, wherein the amino acid substitution is selected from the group consisting of N434A, N434H, N434F, N434S, N434W, and N434Y; and
(x) a position that corresponds to amino acid position 436 of a wild type IgG of the livestock animal, wherein the amino acid substitution is Y436H.

In some embodiments, the polypeptide comprises an amino acid substitution at at least one position selected from the group consisting of:
(i) a position that corresponds to amino acid position 252 of a wild type IgG of the livestock animal, wherein the amino acid substitution is T252Y;
(ii) a position that corresponds to amino acid position 309 of a wild type IgG of the livestock animal, wherein the amino acid substitution is Q309V;
(iii) a position that corresponds to amino acid position 311 of a wild type IgG of the livestock animal, wherein the amino acid substitution is Q311V;
(iv) a position that corresponds to amino acid position 434 of a wild type IgG of the livestock animal, wherein the amino acid substitution is selected from the group consisting of N434Y; and
(v) a position that corresponds to amino acid position 436 of a wild type IgG of the livestock animal, wherein the amino acid substitution is selected from the group consisting of Y436H.

In some embodiments, the wild type IgG Fc is a bovine IgG Fc comprising an amino acid sequence selected from the group consisting of SEQ ID NOs: 12 to 14.

In some embodiments, the livestock animal is an equine.

In some embodiments, the polypeptide comprises an amino acid substitution at at least one position selected from the group consisting of:
(i) a position that corresponds to amino acid position 250 of a wild type IgG of the livestock animal, wherein the amino acid substitution is selected from the group consisting of T250Q, T250E, A250Q, A250E, V250Q, and V250E;
(ii) a position that corresponds to amino acid position 252 of a wild type IgG of the livestock animal, wherein the amino acid substitution is selected from the group consisting of M252Y, M252W, M252F, K252Y, K252W, and K252F;
(iii) a position that corresponds to amino acid position 254 of a wild type IgG of the livestock animal, wherein the amino acid substitution is selected from the group consisting of S254T, S254R, S254K, S254H, T254R, T254K, and T254H;
(iv) a position that corresponds to amino acid position 256 of a wild type IgG of the livestock animal, wherein the amino acid substitution is selected from the group consisting of T256A, T256D, T256E, M256A, M256D, M256E, K256A, K256D, and K256E;
(v) a position that corresponds to amino acid position 286 of a wild type IgG of the livestock animal, wherein the amino acid substitution is selected from the group consisting of S286Y, S286F, S286W, S286L, S286D, S286E, T286Y, T286F, T286W, T286L, T286D, and T286E;
(vi) a position that corresponds to amino acid position 309 of a wild type IgG of the livestock animal, wherein the amino acid substitution is selected from the group consisting of Q309V, Q309D, and Q309E;
(vii) a position that corresponds to amino acid position 311 of a wild type IgG of the livestock animal, wherein the amino acid substitution is selected from the group consisting of Q311V, Q311K, Q311R, Q311L, Q311H, K311V, K311R, K311L, and K311H;
(viii) a position that corresponds to amino acid position 426 of a wild type IgG of the livestock animal, wherein the amino acid substitution is selected from the group consisting of A426Y, A426F, A426H, V426Y, V426F, V426H, G426Y, G426F, and G426H;
(ix) a position that corresponds to amino acid position 428 of a wild type IgG of the livestock animal, wherein the amino acid substitution is selected from the group consisting of M428L, M428Y, and M428F;
(x) a position that corresponds to amino acid position 434 of a wild type IgG of the livestock animal, wherein the amino acid substitution is selected from the group consisting of N434A, N434H, N434F, N434S, N434W, N434Y, H434A, H434F, H434S, H434W, and H434Y; and
(xi) a position that corresponds to amino acid position 436 of a wild type IgG of the livestock animal, wherein the amino acid substitution is selected from the group consisting of Y436H, F436H, and V436H.

In some embodiments, the polypeptide comprises an amino acid substitution at at least one position selected from the group consisting of:
(i) a position that corresponds to amino acid position 252 of a wild type IgG of the livestock animal, wherein the amino acid substitution is M252Y;
(ii) a position that corresponds to amino acid position 286 of a wild type IgG of the livestock animal, wherein the amino acid substitution is T286E
(iii) a position that corresponds to amino acid position 309 of a wild type IgG of the livestock animal, wherein the amino acid substitution is Q309V;
(iv) a position that corresponds to amino acid position 311 of a wild type IgG of the livestock animal, wherein the amino acid substitution is Q311V;
(v) a position that corresponds to amino acid position 426 of a wild type IgG of the livestock animal, wherein the amino acid substitution is G426Y; and
(vi) a position that corresponds to amino acid position 434 of a wild type IgG of the livestock animal, wherein the amino acid substitution is selected from the group consisting of N434Y.

In some embodiments, the wild type IgG Fc is an equine IgG Fc comprising an amino acid sequence selected from the group consisting of SEQ ID NOs: 15 to 21.

In some embodiments, the livestock animal is an ovine.

In some embodiments, the polypeptide comprises an amino acid substitution at at least one position selected from the group consisting of:
(i) a position that corresponds to amino acid position 250 of a wild type IgG of the livestock animal, wherein the amino acid substitution is selected from the group consisting of S250Q, S250E, T250Q, and T250E;
(ii) a position that corresponds to amino acid position 252 of a wild type IgG of the livestock animal, wherein the amino acid substitution is selected from the group consisting of M252Y, M252W, M252F, T252Y, T252W, and T252F;
(iii) a position that corresponds to amino acid position 254 of a wild type IgG of the livestock animal, wherein the amino acid substitution is selected from the group consisting of S254R, S254K, S254H, T254R, T254K, and T254H;
(iv) a position that corresponds to amino acid position 256 of a wild type IgG of the livestock animal, wherein the amino acid substitution is selected from the group consisting of T256A, T256D, and T256E;
(v) a position that corresponds to amino acid position 286 of a wild type IgG of the livestock animal, wherein the amino acid substitution is selected from the group consisting of T286Y, T286F, T286W, T286L, T286D, and T286E;
(vi) a position that corresponds to amino acid position 309 of a wild type IgG of the livestock animal, wherein the amino acid substitution is selected from the group consisting of Q309V, Q309D, and Q309E;
(vii) a position that corresponds to amino acid position 311 of a wild type IgG of the livestock animal, wherein the amino acid substitution is selected from the group consisting of Q311V, Q311K, Q311R, Q311L, Q311H, D311V, D311K, D311R, D311L, and D311H;
(viii) a position that corresponds to amino acid position 426 of a wild type IgG of the livestock animal, wherein the amino acid substitution is selected from the group consisting of V426Y, V426F, and V426H;
(ix) a position that corresponds to amino acid position 428 of a wild type IgG of the livestock animal, wherein the amino acid substitution is selected from the group consisting of M428L, M428Y, and M428F;
(x) a position that corresponds to amino acid position 434 of a wild type IgG of the livestock animal, wherein the amino acid substitution is selected from the group consisting of N434A, N434H, N434F, N434S, N434W, and N434Y; and
(xi) a position that corresponds to amino acid position 436 of a wild type IgG of the livestock animal, wherein the amino acid substitution is Y436H.

In some embodiments, the wild type IgG Fc is an ovine IgG Fc comprising an amino acid sequence selected from the group consisting of SEQ ID NOs: 22 to 24.

In some embodiments, the polypeptide comprises an amino acid substitution at at least one position selected from the group consisting of:
(i) a position that corresponds to amino acid position 252 of a wild type IgG of the livestock animal, wherein the amino acid substitution is T252Y;
(ii) a position that corresponds to amino acid position 309 of a wild type IgG of the livestock animal, wherein the amino acid substitution is Q309V;
(iii) a position that corresponds to amino acid position 311 of a wild type IgG of the livestock animal, wherein the amino acid substitution is Q311V;
(iv) a position that corresponds to amino acid position 428 of a wild type IgG of the livestock animal, wherein the amino acid substitution is selected from the group consisting of M428Y; and
(v) a position that corresponds to amino acid position 434 of a wild type IgG of the livestock animal, wherein the amino acid substitution is selected from the group consisting of N434Y.

In some embodiments, the wild type IgG Fc is an ovine IgG Fc comprising an amino acid sequence selected from the group consisting of SEQ ID NOs: 22 to 24.

In some embodiments, the livestock animal is a caprine.

In some embodiments, the polypeptide comprises an amino acid substitution at at least one position selected from the group consisting of:
(i) a position that corresponds to amino acid position 250 of a wild type IgG of the livestock animal, wherein the amino acid substitution is selected from the group consisting of S250Q, S250E, T250Q, and T250E;
(ii) a position that corresponds to amino acid position 252 of a wild type IgG of the livestock animal, wherein the amino acid substitution is selected from the group consisting of M252Y, M252W, M252F, T252Y, T252W, and T252F;
(iii) a position that corresponds to amino acid position 254 of a wild type IgG of the livestock animal, wherein the amino acid substitution is selected from the group consisting of S254R, S254K, S254H, T254R, T254K, and T254H;
(iv) a position that corresponds to amino acid position 256 of a wild type IgG of the livestock animal, wherein the amino acid substitution is selected from the group consisting of T256A, T256D, T256E, K256A, K256D, and K256E;
(v) a position that corresponds to amino acid position 286 of a wild type IgG of the livestock animal, wherein the amino acid substitution is selected from the group consisting of T286Y, T286F, T286W, T286L, T286D, and T286E;
(vi) a position that corresponds to amino acid position 309 of a wild type IgG of the livestock animal, wherein the amino acid substitution is selected from the group consisting of Q309V, Q309D, and Q309E;
(vii) a position that corresponds to amino acid position 311 of a wild type IgG of the livestock animal, wherein the amino acid substitution is selected from the group consisting of Q311V, Q311K, Q311R, Q311L, Q311H, K311V, K311R, K311L, K311H, D311V, D311K, D311R, D311L, and D311H;
(viii) a position that corresponds to amino acid position 426 of a wild type IgG of the livestock animal, wherein the amino acid substitution is selected from the group consisting of A426Y, A426F, A426H, V426Y, V426F, and V426H;
(ix) a position that corresponds to amino acid position 428 of a wild type IgG of the livestock animal, wherein the amino acid substitution is selected from the group consisting of M428L, M428Y, and M428F;
(x) a position that corresponds to amino acid position 434 of a wild type IgG of the livestock animal, wherein the amino acid substitution is selected from the group consisting of N434A, N434H, N434F, N434S, N434W, and N434Y; and
(xi) a position that corresponds to amino acid position 436 of a wild type IgG of the livestock animal, wherein the amino acid substitution is Y436H.

In some embodiments, the wild type IgG Fc is a caprine IgG Fc comprising an amino acid sequence selected from the group consisting of SEQ ID NOs: 25 to 27.

In some embodiments, the polypeptide comprises at two or more of the at least one amino acid substitutions, wherein the two or more amino acid substitutions are at different positions, and wherein the polypeptide has increased binding affinity to an FcRn of the livestock animal when compared to (a) an Fc domain of the wild type IgG of the livestock animal, and (b) a polypeptide comprising only one of the two or more amino acid substitutions.

In some embodiments, the polypeptide further comprises a binding domain. In some embodiments, the binding domain comprises (i) six complementarity determining regions (CDRs) of an immunoglobulin molecule; (ii) a ligand binding domain of a receptor protein of the livestock animal; (iii) a nanobody; or (iv) an extracellular domain of a receptor protein of the livestock animal.

In some embodiments, the binding domain specifically binds to an antigen selected from the group consisting of NGF, TrKA, ADAMTS, IL-1, IL-2, IL-4, IL-4R, Angiotensin type 1 (AT1) receptor, Angiotensin type 2 (AT2) receptor, IL-5, IL-12, IL-13, IL-31, IL-33, CD3, CD20, CD47, CD52, and complement system complex.

In some embodiments, a specific binding between a binding domain (e.g., a binding domain of an antibody) and an antigen is represented by a K_{D} value. K_{D} is the ratio of the antibody dissociation rate (k_{off}) to the antibody association rate (kₒₙ) of the antibody. The K_{D} values can be determined by measuring the kₒₙ and k_{off} rates of a specific antibody/antigen interaction and then using a ratio of these values to calculate the K_{D} value. The smaller the K_{D} value the greater the affinity of the antibody (or a binding domain thereof) for its target. In some embodiments, the K_{D} value for a specific binding ranges from about 10⁻⁶ to about 10⁻¹² (e.g., 10⁻⁶, 10⁻⁷, 10⁻⁸, 10⁻⁹, 10⁻¹⁰, 10⁻¹¹, or 10⁻¹²).

In some embodiments, the polypeptide further comprises a protein selected from the group consisting of EPO, CTLA4, LFA3, VEGFR1/VEGFR3, IL-1R, IL-4R, GLP-1 receptor agonist, and Thrombopoietin binding peptide.

In some embodiments, the polypeptide binds to an FcRn of the livestock animal at a higher level at an acidic pH than at a neutral pH.

In some embodiments, the polypeptide binds to an FcRn of the livestock animal at a higher level at pH 5.5 than at pH 7.4.

In some embodiments, the polypeptide binds to an FcRn of the livestock animal at a higher level at pH 6.0 than at pH 7.4.

Also disclosed herein is a pharmaceutical composition comprising (i) the polypeptide as described herein, and (ii) a pharmaceutically acceptable excipient.

The present disclosure also extends to a nucleic acid or nucleic acids encoding the polypeptide as described herein.

In another aspect disclosed herein, there is provided an expression vector or expression vectors comprising the nucleic acid or nucleic acids as described herein. The present disclosure also extends to a host cell comprising the nucleic acid or nucleic acids disclosed herein or the expression vector or expression vectors disclosed herein.

Also disclosed herein is a method of making a polypeptide, the method comprising:
(a) providing a nucleic acid or nucleic acids as disclosed herein;
(b) expressing the nucleic acid or nucleic acids in a host cell culture, thereby producing the polypeptide; and
(c) collecting the polypeptide produced in (b) from the host cell culture.

In some embodiments, the method further comprises formulating the polypeptide as a pharmaceutical formulation.

The present disclosure also extends to a method of treating a disease or disorder in a livestock animal in need thereof, the method comprising administering an effective amount of a composition comprising the pharmaceutical composition as disclosed herein to the livestock animal.

The present disclosure also extends to a method of preventing a disease or disorder in a livestock animal in need thereof, the method comprising administering an effective amount of a composition comprising the pharmaceutical composition as disclosed herein to the livestock animal.

In some embodiments, the disease or disorder is an allergy, chronic pain, acute pain, an inflammatory disease, an autoimmune disease, an endocrine disease, a gastrointestinal disease, a cardiovascular disease, a renal disease, a fertility related disorder, an infectious disease, or a cancer.

In some embodiments, the disease or disorder is atopic dermatitis, allergic dermatitis, osteoarthritic pain, arthritis, anemia, or obesity.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

Other features and advantages of the invention will be apparent from the following detailed description and from the claims.

### BRIEF DESCRIPTION OF THE DRAWINGS

FIG. 1 shows the alignment of the amino acid sequences of the wild-type Fc regions of porcine IgG1a, ovine IgG1, bovine IgG1, equine IgG1, feline IgG1a, canine IgGB, and human IgG1. These sequences are assigned SEQ ID NOs: 30-36, respectively.

FIGS. 2A-2J show the purity of the porcine antibodies with the Fc variants determined by scanning densitometry of Coomassie blue-stained SDS/PAGE gels. The purity of each antibody was >90%.

FIGS. 3A-3J show the purity of the ovine antibodies with the Fc variants determined by scanning densitometry of Coomassie blue-stained SDS/PAGE gels. The purity of each antibody was >90%.

FIGS. 4A-4J show the purity of the bovine antibodies with the Fc variants determined by scanning densitometry of Coomassie blue-stained SDS/PAGE gels. The purity of each antibody was >90%.

FIGS. 5A-5J show the purity of the equine antibodies with the Fc variants determined by scanning densitometry of Coomassie blue-stained SDS/PAGE gels. The purity of each antibody was >90%.

FIGS. 6A-6J show the sensorgrams of kinetic binding data for the porcine IgG variants and wild-type.

FIGS. 7A-7J show the sensorgrams of kinetic binding data for the ovine IgG variants and wild-type.

FIGS. 8A-8J show the sensorgrams of kinetic binding data for the bovine IgG variants and wild-type.

FIGS. 9A-9J show the sensorgrams of kinetic binding data for the equine IgG variants and wild-type.

### DETAILED DESCRIPTION

With the increasing use of polypeptide (e.g., antibodies, ligand-binding domains of receptors, enzymes, ligands, peptides) as therapeutics for the prevention and treatment of a wide variety of diseases that affect livestock animals, it is important to develop polypeptides with extended half-life, especially for the prevention or treatment of chronic diseases in which a polypeptide must be administered repetitively.

Accordingly, this disclosure features immunoglobulin Fc regions of livestock animals or FcRn-binding regions thereof comprising mutations that enhance the half-life of the polypeptide or polypeptides comprising these sequences in the livestock animal. Also disclosed are polypeptides comprising these domains and methods of their use. These peptides can be used for various therapeutic and diagnostic purposes.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated. All numerical designations, e.g., pH, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

The term “about,” as used herein when referring to a measurable value such as an amount or concentration and the like, is meant to encompass variations of 20%, 10%, 5%, 1%, 0.5%, or even 0.1% of the specified amount.

### Porcine Antibodies

Pigs typically have eleven IgG heavy chains referred to as IgG1a, IgG1b, IgG2a, IgG2b, IgG3, IgG4a, IgG4b, IgG5a, IgG5b, IgG6a, and IgG6b. These heavy chains represent eleven different subclasses of porcine IgG. The amino acid and DNA sequences for these heavy chains are available from the GENBANK® database. Illustrative examples of the amino acid sequences of the CH2 and CH3 domains of each of the eleven porcine IgG heavy chain Fc regions are shown below (CH2 domains are underlined), and the GENBANK® accession number (for the amino acid sequence or mRNA sequence from which the amino acid sequence is derived) of each heavy chain Fc region is indicated:

Porcine (Sus scrofa) IgG Fc sequences
IgG1a (GenBank®® AAA52219.1)
(SEQ ID NO: 1)
GCEVAGPSVFIFPPKPKDTLMISQTPEVTCVVVDVSKEHAEVQFSWYVDGV
EVHTAETRPKEEQFNSTYRVVSVLPIQHQDWLKGKEFKCKVNNVDLPAPIT
RTISKAIGQSREPQVYTLPPPAEELSRSKVTVTCLVIGFYPPDIHVEWKSN
GQPEPEGNYRTTPPQQDVDGTFFLYSKLAVDKARWDHGETFECAVMHEALH
NHYTQKSISKTQGK
IgG1b (GenBank® AAA52216.1)
(SEQ ID NO: 2)
GCEVAGPSVFIFPPKPKDTLMISQTPEVTCVVVDVSKEHAEVQFSWYVDGV
EVHTAETRPKEEQFNSTYRVVSVLPIQHQDWLKGKEFKCKVNNVDLPAPIT
RTISKAIGQSREPQVYTLPPPAEELSRSKVTLTCLVIGFYPPDIHVEWKSN
GQPEPENTYRTTPPQQDVDGTFFLYSKLAVDKARWDHGDKFECAVMHEALH
NHYTQKSISKTQGK
IgG2a (GenBank® U03779)
(SEQ ID NO: 3)
ACESPGPSVFIFPPKPKDTLMISRTPQVTCVVVDVSQENPEVQFSWYVDGV
EVHTAQTRPKEEQFNSTYRVVSVLPIQHQDWLNGKEFKCKVNNKDLPAPIT
RIISKAKGQTREPQVYTLPPHAEELSRSKVSITCLVIGFYPPDIDVEWQRN
GQPEPEGNYRTTPPQQDVDGTYFLYSKFSVDKASWQGGGIFQCAVMHEALH
NHYTQKSISKTPGK
IgG2b (GenBank® U03780)
(SEQ ID NO: 4)
ACESPGPSVFIFPPKPKDTLMISRTPQVTCVVVDVSQENPEVQFSWYVDGV
EVHTAQTRPKEEQFNSTYRVVSVLPIQHQDWLNGKEFKCKVNNKDLPAPIT
RIISKAKGQTREPQVYTLPPHAEELSRSKVSITCLVIGFYPPDIDVEWQRN
GQPEPEGNYRTTPPQQDVDGTYFLYSKFSVDKASWQGGGIFQCAVMHEALH
NHYTQKSISKTPGK
IgG3 (GenBank® ABY85810.1)
(SEQ ID NO: 5)
AAEVLGAPSVFLFPPKPKDILMISRTPKVTCVVVDVSQEEAEVQFSWYVDG
VQLYTAQTRPMEEQFNSTYRVVSVLPIQHQDWLKGKEFKCKVNNKDLLSPI
TRTISKATGPSRVPQVYTLPPAWEELSKSKVSITCLVTGFYPPDIDVEWQS
NGQQEPEGNYRTTPPQQDVDGTYFLYSKLAVDKVRWQRGDLFQCAVMHEAL
HNHYTQKSISKTQGK
IgG4a (GenBank® AAA52220.1)
(SEQ ID NO: 6)
ACEGPGPSAFIFPPKPKDTLMISRTPKVTCVVVDVSQENPEVQFSWYVDGV
EVHTAQTRPKEEQFNSTYRVVSVLPIQHQDWLNGKEFKCKVNNKDLPAPIT
RIISKAKGQTREPQVYTLPPPTEELSRSKVTLTCLVTGFYPPDIDVEWQRN
GQPEPEGNYRTTPPQQDVDGTYFLYSKLAVDKASWQRGDTFQCAVMHEALH
NHYTQKSIFKTPGK
IgG4b (GenBank® ABY85806.1)
(SEQ ID NO: 7)
ACEGPGPSAFIFPPKPKDTLMISRTPKVTCVVVDVSQENPEVQFSWYVDGV
EVHTAQTRPKEEQFNSTYRVVSVLLIQHQDWLNGKEFKCKVNNKDLPAPIT
RIISKAKGQTREPQVYTLPPPTEELSRSKVTLTCLVTGFYPPDIDVEWQRN
GQPEPEGNYRTTPPQQDVDGTYFLYSKLAVDKASWQRGDTFQCAVMHEALH
NHYT
IgG5a (GenBank® ABY85809.1)
(SEQ ID NO: 8)
GCEVAGPSVFIFPPKPKDILMISRTPEVTCVVVDVSKEHAEVQFSWYVDGE
EVHTAETRPKEEQFNSTYRVVSVLPIQHEDWLKGKEFECKVNNEDLPGPIT
RTISKAKGVVRSPEVYTLPPPAEELSKSIVTLTCLVKSIFPPFIHVEWKIN
GKPEPENAYRTTPPQEDEDRTYFLYSKLAVDKARWDHGETFECAVMHEALH
NHYTQKSISKTQGK
IgG5b (GenBank® AB699687)
(SEQ ID NO: 9)
ICPVAGPSVFIFPPKPKDILMISRTPEVTCVVVDVSKEHAEVQFSWYVDGE
EVHTAETRPKEEQFNSTYRVVSVLPIQHEDWLKGKEFECKVNNEDLPGPIT
RTISKAKGVVRSPEVYTLPPPAEELSKSIVTLTCLVKSFFPPFIHVEWKIN
GKPEPENAYRTTPPQEDEDGTYFLYSKFSVEKFRWHSGGIHCAVMHEALHN
HYTEKSVSQTPGK
IgG6a (GenBank® AB699687)
(SEQ ID NO: 10)
ACEGPGPSAFIFPPKPKDTLMISRTPKVTCVVVDVSQENPEVQFSWYVDGV
EVHTAQTRPKEEQFNSTYRVVSVLPIQHQDWLNGKEFKCKVNNKDLPAPIT
RIISKAKGQTREPQVYTLPPPTEELSRSKLSVTCLITGFYPPDIDVEWQRN
GQPEPEGNYRTTPPQQDVDGTYFLYSKLAVDKASWQRGDPFQCAVMHEALH
NHYTQKSIFKTPGN
IgG6b (GenBank® ABY85805.1)
(SEQ ID NO: 11)
ACEGNGPSVFIFPPKPKDTLMISRTPEVTCVVVDVSQENPEVQFSWYVDGE
EVHTAETRPKEEQFNSTYRVVSVLPIQHQDWLKGKEFECKVNNKDLPAPIT
RIISKAKGPSREPQVYTLSPSAEELSRSKVSITCLVTGFYPPDIDVEWKSN
GQPEPEGNYRSTPPQEDEDGTYFLYSKLAVDKARLQSGGIHCAVMHEALHN
HYTQKSISKT

The CH2 region of a porcine antibody comprises or consists of amino acids 231 to 340 (according to EU numbering) of a porcine IgG antibody. It is to be understood that the CH2 region may include one to six (e.g., 1, 2, 3, 4, 5, 6) additional amino acids or deletions at their N and/or C-terminus.

The CH3 region of a porcine antibody comprises or consists of amino acids 341 to 447 (according to EU numbering) of a porcine IgG antibody. It is to be understood that the CH3 region may include one to six (e.g., 1, 2, 3, 4, 5, 6) additional amino acids or deletions at their N and/or C-terminus.

The Fc region of a porcine IgG antibody comprises or consists of amino acids 231 to 447 (according to EU numbering) of a porcine IgG antibody.

Table 1 below compares the amino acid sequences of the CH2 and CH3 domains of human IgG1, canine IgGB and feline IgG1a with each of the eleven porcine IgG isotypes, based on EU numbering:

TABLE 1

| | CH2 Domain | | | | | | | | | | | | | |
|---|---|---|---|---|---|---|---|---|---|---|---|---|---|---|
| EU | Human | Canine | Feline | Pig | Pig | Pig | Pig | Pig | Pig | Pig | Pig | Pig | Pig | Pig |
| number | IgG1 | IgGB | IgG1a | IgG1a | IgG1b | IgG2a | IgG2b | IgG3 | IgG4a | IgG4b | IgG5a | IgG5b | IgG6a | IgG6b |
| 231 | A | A | P | | | | | A | | | | | | |
| 232 | P | P | P | G | G | A | A | A | A | A | G | I | A | A |
| 233 | E | E | E | C | C | C | C | E | C | C | C | C | C | C |
| 234 | L | M | M | E | E | E | E | V | E | E | E | P | E | E |
| 235 | L | L | L | V | V | S | S | L | G | G | V | V | G | G |
| 236 | G | G | G | A | A | P | P | G | P | P | A | A | P | N |
| 237 | G | G | G | G | G | G | G | A | G | G | G | G | G | G |
| 238 | P | P | P | P | P | P | P | P | P | P | P | P | P | P |
| 239 | S | S | S | S | S | S | S | S | S | S | S | S | S | S |
| 240 | V | V | I | V | V | V | V | V | A | A | V | V | A | V |
| 241 | F | F | F | F | F | F | F | F | F | F | F | F | F | F |
| 242 | L | I | I | I | I | I | I | L | I | I | I | I | I | I |
| 243 | F | F | F | F | F | F | F | F | F | F | F | F | F | F |
| 244 | P | P | P | P | P | P | P | P | P | P | P | P | P | P |
| 245 | P | P | P | P | P | P | P | P | P | P | P | P | P | P |
| 246 | K | K | K | K | K | K | K | K | K | K | K | K | K | K |
| 247 | P | P | P | P | P | P | P | P | P | P | P | P | P | P |
| 248 | K | K | K | K | K | K | K | K | K | K | K | K | K | K |
| 249 | D | D | D | D | D | D | D | D | D | D | D | D | D | D |
| 250 | T | T | T | T | T | T | T | I | T | T | I | I | T | T |
| 251 | L | L | L | L | L | L | L | L | L | L | L | L | L | L |
| 252 | M | L | S | M | M | M | M | M | M | M | M | M | M | M |
| 253 | I | I | I | I | I | I | I | I | I | I | I | I | I | I |
| 254 | S | A | S | S | S | S | S | S | S | S | S | S | S | S |
| 255 | R | R | R | Q | Q | R | R | R | R | R | R | R | R | R |
| 256 | T | T | T | T | T | T | T | T | T | T | T | T | T | T |
| 257 | P | P | P | P | P | P | P | P | P | P | P | P | P | P |
| 258 | E | E | E | E | E | Q | Q | K | K | K | E | E | K | E |
| 259 | V | V | V | V | V | V | V | V | V | V | V | V | V | V |
| 260 | T | T | T | T | T | T | T | T | T | T | T | T | T | T |
| 261 | C | C | C | C | C | C | C | C | C | C | C | C | C | C |
| 262 | V | V | L | V | V | V | V | V | V | V | V | V | V | V |
| 263 | V | V | V | V | V | V | V | V | V | V | V | V | V | V |
| 264 | V | V | V | V | V | V | V | V | V | V | V | V | V | V |
| 265 | D | D | D | D | D | D | D | D | D | D | D | D | D | D |
| 266 | V | L | L | V | V | V | V | V | V | V | V | V | V | V |
| 267 | S | D | G | S | S | S | S | S | S | S | S | S | S | S |
| 268 | H | P | P | K | K | Q | Q | Q | Q | Q | K | K | Q | Q |
| 269 | E | E | D | E | E | E | E | E | E | E | E | E | E | E |
| 270 | D | D | D | H | H | N | N | E | N | N | H | H | N | N |
| 271 | P | P | S | A | A | P | P | A | P | P | A | A | P | P |
| 272 | E | E | D | E | E | E | E | E | E | E | E | E | E | E |
| 273 | V | V | V | V | V | V | V | V | V | V | V | V | V | V |
| 274 | K | Q | Q | Q | Q | Q | Q | Q | Q | Q | Q | Q | Q | Q |
| 275 | F | I | I | F | F | F | F | F | F | F | F | F | F | F |
| 276 | N | S | T | S | S | S | S | S | S | S | S | S | S | S |
| 277 | W | W | W | W | W | W | W | W | W | W | W | W | W | W |
| 278 | Y | F | F | Y | Y | Y | Y | Y | Y | Y | Y | Y | Y | Y |
| 279 | V | V | V | V | V | V | V | V | V | V | V | V | V | V |
| 280 | D | D | D | D | D | D | D | D | D | D | D | D | D | D |
| 282 | G | G | N | G | G | G | G | G | G | G | G | G | G | G |
| 282 | V | K | T | V | V | V | V | V | V | V | E | E | V | E |
| 283 | E | Q | Q | E | E | E | E | Q | E | E | E | E | E | E |
| 284 | V | M | V | V | V | V | V | L | V | V | V | V | V | V |
| 285 | H | Q | Y | H | H | H | H | Y | H | H | H | H | H | H |
| 286 | N | T | T | T | T | T | T | T | T | T | T | T | T | T |
| 287 | A | A | A | A | A | A | A | A | A | A | A | A | A | A |
| 288 | K | K | K | E | E | Q | Q | Q | Q | Q | E | E | Q | E |
| 289 | T | T | T | T | T | T | T | T | T | T | T | T | T | T |
| 290 | K | Q | S | R | R | R | R | R | R | R | R | R | R | R |
| 291 | P | P | P | P | P | P | P | P | P | P | P | P | P | P |
| 292 | R | R | R | K | K | K | K | M | K | K | K | K | K | K |
| 293 | E | E | E | E | E | E | E | E | E | E | E | E | E | E |
| 294 | E | E | E | E | E | E | E | E | E | E | E | E | E | E |
| 295 | Q | Q | Q | Q | Q | Q | Q | Q | Q | Q | Q | Q | Q | Q |
| 296 | Y | F | F | F | F | F | F | F | F | F | F | F | F | F |
| 297 | N | N | N | N | N | N | N | N | N | N | N | N | N | N |
| 298 | S | G | S | S | S | S | S | S | S | S | S | S | S | S |
| 299 | T | T | T | T | T | T | T | T | T | T | T | T | T | T |
| 300 | Y | Y | Y | Y | Y | Y | Y | Y | Y | Y | Y | Y | Y | Y |
| 301 | R | R | R | R | R | R | R | R | R | R | R | R | R | R |
| 302 | V | V | V | V | V | V | V | V | V | V | V | V | V | V |
| 303 | V | V | V | V | V | V | V | V | V | V | V | V | V | V |
| 304 | S | S | S | S | S | S | S | S | S | S | S | S | S | S |
| 305 | V | V | V | V | V | V | V | V | V | V | V | V | V | V |
| 306 | L | L | L | L | L | L | L | L | L | L | L | L | L | L |
| 307 | T | P | P | P | P | P | P | P | P | L | P | P | P | P |
| 308 | V | I | I | I | I | I | I | I | I | I | I | I | I | I |
| 309 | L | G | L | Q | Q | Q | Q | Q | Q | Q | Q | Q | Q | Q |
| 310 | H | H | H | H | H | H | H | H | H | H | H | H | H | H |
| 311 | Q | Q | Q | Q | Q | Q | Q | Q | Q | Q | E | E | Q | Q |
| 312 | D | D | D | D | D | D | D | D | D | D | D | D | D | D |
| 313 | W | W | W | W | W | W | W | W | W | W | W | W | W | W |
| 314 | L | L | L | L | L | L | L | L | L | L | L | L | L | L |
| 315 | N | K | K | K | K | N | N | K | N | N | K | K | N | K |
| 316 | G | G | G | G | G | G | G | G | G | G | G | G | G | G |
| 317 | K | K | K | K | K | K | K | K | K | K | K | K | K | K |
| 318 | E | Q | E | E | E | E | E | E | E | E | E | E | E | E |
| 319 | Y | F | F | F | F | F | F | F | F | F | F | F | F | F |
| 320 | K | T | K | K | K | K | K | K | K | K | E | E | K | E |
| 321 | C | C | C | C | C | C | C | C | C | C | C | C | C | C |
| 322 | K | K | K | K | K | K | K | K | K | K | K | K | K | K |
| 323 | V | V | V | V | V | V | V | V | V | V | V | V | V | V |
| 324 | S | N | N | N | N | N | N | N | N | N | N | N | N | N |
| 325 | N | N | S | N | N | N | N | N | N | N | N | N | N | N |
| 326 | K | K | K | V | V | K | K | K | K | K | E | E | K | K |
| 327 | A | A | S | D | D | D | D | D | D | D | D | D | D | D |
| 328 | L | L | L | L | L | L | L | L | L | L | L | L | L | L |
| 329 | P | P | P | P | P | P | P | L | P | P | P | P | P | P |
| 330 | A | S | S | A | A | A | A | S | A | A | G | G | A | A |
| 331 | P | P | P | P | P | P | P | P | P | P | P | P | P | P |
| 332 | I | I | I | I | I | I | I | I | I | I | I | I | I | I |
| 333 | E | E | E | T | T | T | T | T | T | T | T | T | T | T |
| 334 | K | R | R | R | R | R | R | R | R | R | R | R | R | R |
| 335 | T | T | T | T | T | I | I | T | I | I | T | T | I | I |
| 336 | I | I | I | I | I | I | I | I | I | I | I | I | I | I |
| 337 | S | S | S | S | S | S | S | S | S | S | S | S | S | S |
| 338 | K | K | K | K | K | K | K | K | K | K | K | K | K | K |
| 339 | A | A | A | A | A | A | A | A | A | A | A | A | A | A |
| 340 | K | R | K | I | I | K | K | T | K | K | K | K | K | K |
| | CH3 Domain | | | | | | | | | | | | | |
| EU | Human | Canine | Feline | Pig | Pig | Pig | Pig | Pig | Pig | Pig | Pig | Pig | Pig | Pig |
| number | IgG1 | IgGB | IgG1a | IgG1a | IgG1b | IgG2a | IgG2b | IgG3 | IgG4a | IgG4b | IgG5a | IgG5b | IgG6a | IgG6b |
| 341 | G | G | G | G | G | G | G | G | G | G | G | G | G | G |
| 342 | Q | Q | Q | Q | Q | Q | Q | P | Q | Q | V | V | Q | P |
| 343 | P | A | P | S | S | T | T | S | T | T | V | V | T | S |
| 344 | R | H | H | R | R | R | R | R | R | R | R | R | R | R |
| 345 | E | Q | E | E | E | E | E | V | E | E | S | S | E | E |
| 346 | P | P | P | P | P | P | P | P | P | P | P | P | P | P |
| 347 | Q | S | Q | Q | Q | Q | Q | Q | Q | Q | E | E | Q | Q |
| 348 | V | V | V | V | V | V | V | V | V | V | V | V | V | V |
| 349 | Y | Y | Y | Y | Y | Y | Y | Y | Y | Y | Y | Y | Y | Y |
| 350 | T | V | V | T | T | T | T | T | T | T | T | T | T | T |
| 351 | L | L | L | L | L | L | L | L | L | L | L | L | L | L |
| 352 | P | P | P | P | P | P | P | P | P | P | P | P | P | S |
| 353 | P | P | P | P | P | P | P | P | P | P | P | P | P | P |
| 354 | S | S | A | P | P | H | H | A | P | P | P | P | P | S |
| 355 | R | R | Q | A | A | A | A | W | T | T | A | A | T | A |
| 356 | D | E | E | E | E | E | E | E | E | E | E | E | E | E |
| 357 | E | E | E | E | E | E | E | E | E | E | E | E | E | E |
| 358 | L | L | L | L | L | L | L | L | L | L | L | L | L | L |
| 359 | T | S | S | S | S | S | S | S | S | S | S | S | S | S |
| 360 | K | K | R | R | R | R | R | K | R | R | K | K | R | R |
| 361 | N | N | N | S | S | S | S | S | S | S | S | S | S | S |
| 362 | Q | T | K | K | K | K | K | K | K | K | I | I | K | K |
| 363 | V | V | V | V | V | V | V | V | V | V | V | V | L | V |
| 364 | S | S | S | T | T | S | S | S | T | T | T | T | S | S |
| 365 | L | L | V | V | L | I | I | I | L | L | L | L | V | I |
| 366 | T | T | T | T | T | T | T | T | T | T | T | T | T | T |
| 367 | C | C | C | C | C | C | C | C | C | C | C | C | C | C |
| 368 | L | L | L | L | L | L | L | L | L | L | L | L | L | L |
| 369 | V | I | I | V | V | V | V | V | V | V | V | V | I | V |
| 370 | K | K | K | I | I | I | I | T | T | T | K | K | T | T |
| 371 | G | D | S | G | G | G | G | G | G | G | S | S | G | G |
| 372 | F | F | F | F | F | F | F | F | F | F | I | F | F | F |
| 373 | Y | F | H | Y | Y | Y | Y | Y | Y | Y | F | F | Y | Y |
| 374 | P | P | P | P | P | P | P | P | P | P | P | P | P | P |
| 375 | S | P | P | P | P | P | P | P | P | P | P | P | P | P |
| 376 | D | D | D | D | D | D | D | D | D | D | F | F | D | D |
| 377 | I | I | I | I | I | I | I | I | I | I | I | I | I | I |
| 378 | A | D | A | H | H | D | D | D | D | D | H | H | D | D |
| 379 | V | V | V | V | V | V | V | V | V | V | V | V | V | V |
| 380 | E | E | E | E | E | E | E | E | E | E | E | E | E | E |
| 381 | W | W | W | W | W | W | W | W | W | W | W | W | W | W |
| 382 | E | Q | E | K | K | Q | Q | Q | Q | Q | K | K | Q | K |
| 383 | S | S | I | S | S | R | R | S | R | R | I | I | R | S |
| 384 | N | N | T | N | N | N | N | N | N | N | N | N | N | N |
| 385 | G | G | G | G | G | G | G | G | G | G | G | G | G | G |
| 386 | Q | Q | Q | Q | Q | Q | Q | Q | Q | Q | K | K | Q | Q |
| 387 | P | Q | P | P | P | P | P | Q | P | P | P | P | P | P |
| 388 | E | E | E | E | E | E | E | E | E | E | E | E | E | E |
| 389(a) | N | P | P | P | P | P | P | P | P | P | P | P | P | P |
| 389b | | E | E | E | E | E | E | E | E | E | E | E | E | E |
| 389c | | S | N | G | N | G | G | G | G | G | N | N | G | G |
| 390 | N | K | N | N | T | N | N | N | N | N | A | A | N | N |
| 391 | Y | Y | Y | Y | Y | Y | Y | Y | Y | Y | Y | Y | Y | Y |
| 392 | K | R | R | R | R | R | R | R | R | R | R | R | R | R |
| 393 | T | T | T | T | T | T | T | T | T | T | T | T | T | S |
| 394 | T | T | T | T | T | T | T | T | T | T | T | T | T | T |
| 395 | P | P | P | P | P | P | P | P | P | P | P | P | P | P |
| 396 | P | P | P | P | P | P | P | P | P | P | P | P | P | P |
| 397 | V | Q | Q | Q | Q | Q | Q | Q | Q | Q | Q | Q | Q | Q |
| 398 | L | L | L | Q | Q | Q | Q | Q | Q | Q | E | E | Q | E |
| 399 | D | D | D | D | D | D | D | D | D | D | D | D | D | D |
| 400 | S | E | S | V | V | V | V | V | V | V | E | E | V | E |
| 401 | D | D | D | D | D | D | D | D | D | D | D | D | D | D |
| 402 | G | G | G | G | G | G | G | G | G | G | R | G | G | G |
| 403 | S | S | T | T | T | T | T | T | T | T | T | T | T | T |
| 404 | F | Y | Y | F | F | Y | Y | Y | Y | Y | Y | Y | Y | Y |
| 405 | F | F | F | F | F | F | F | F | F | F | F | F | F | F |
| 406 | L | L | V | L | L | L | L | L | L | L | L | L | L | L |
| 407 | Y | Y | Y | Y | Y | Y | Y | Y | Y | Y | Y | Y | Y | Y |
| 408 | S | S | S | S | S | S | S | S | S | S | S | S | S | S |
| 409 | K | K | K | K | K | K | K | K | K | K | K | K | K | K |
| 410 | L | L | L | L | L | F | F | L | L | L | L | F | L | L |
| 411 | T | S | S | A | A | S | S | A | A | A | A | S | A | A |
| 412 | V | V | V | V | V | V | V | V | V | V | V | V | V | V |
| 413 | D | D | D | D | D | D | D | D | D | D | D | E | D | D |
| 414 | K | K | R | K | K | K | K | K | K | K | K | K | K | K |
| 415 | S | S | S | A | A | A | A | V | A | A | A | F | A | A |
| 416 | R | R | H | R | R | S | S | R | S | S | R | R | S | R |
| 417 | W | W | W | W | W | W | W | W | W | W | W | W | W | L |
| 418 | Q | Q | Q | D | D | Q | Q | Q | Q | Q | D | H | Q | Q |
| 419 | Q | R | R | H | H | G | G | R | R | R | H | S | R | S |
| 420 | G | G | G | G | G | G | G | G | G | G | G | G | G | G |
| 421 | N | D | N | E | D | G | G | D | D | D | E | G | D | G |
| 422 | V | T | T | T | K | I | I | L | T | T | T | I | P | I |
| 423 | F | F | Y | F | F | F | F | F | F | F | F | H | F | H |
| 424 | S | I | T | E | E | Q | Q | Q | Q | Q | E | | Q | C |
| 425 | C | C | C | C | C | C | C | C | C | C | C | C | C | A |
| 426 | S | A | S | A | A | A | A | A | A | A | A | A | A | V |
| 427 | V | V | V | V | V | V | V | V | V | V | V | V | V | M |
| 428 | M | M | S | M | M | M | M | M | M | M | M | M | M | H |
| 429 | H | H | H | H | H | H | H | H | H | H | H | H | H | E |
| 430 | E | E | E | E | E | E | E | E | E | E | E | E | E | A |
| 431 | A | A | A | A | A | A | A | A | A | A | A | A | A | L |
| 432 | L | L | L | L | L | L | L | L | L | L | L | L | L | H |
| 433 | H | H | H | H | H | H | H | H | H | H | H | H | H | N |
| 434 | N | N | S | N | N | N | N | N | N | N | N | N | N | H |
| 435 | H | H | H | H | H | H | H | H | H | H | H | H | H | Y |
| 436 | Y | Y | H | Y | Y | Y | Y | Y | Y | Y | Y | Y | Y | T |
| 437 | T | T | T | T | T | T | T | T | T | T | T | T | T | Q |
| 438 | Q | Q | Q | Q | Q | Q | Q | Q | Q | | Q | E | Q | K |
| 439 | K | E | K | K | K | K | K | K | K | | K | K | K | S |
| 440 | S | S | S | S | S | S | S | S | S | | S | S | S | I |
| 441 | L | L | L | I | I | I | I | I | I | | I | V | I | S |
| 442 | S | S | T | S | S | S | S | S | F | | S | S | F | K |
| 443 | L | H | Q | K | K | K | K | K | K | | K | Q | K | T |
| 444 | S | S | S | T | T | T | T | T | T | | T | T | T | |
| 445 | P | P | P | Q | Q | P | P | Q | P | | Q | P | P | |
| 446 | G | G | G | G | G | G | G | G | G | | G | G | G | |
| 447 | K | K | K | K | K | K | K | K | K | | K | K | N | |

Substitutions in Porcine IgG Fc that Improve Half-Life

Increased serum persistence is a beneficial property for therapeutic polypeptides. This disclosure features substitutions in wild type porcine IgG1a, IgG1b, IgG2a, IgG2b, IgG3, IgG4a, IgG4b, IgG5a, IgG5b, IgG6a, and IgG6b Fc regions that enhance the half-life of a polypeptide or polypeptides comprising these Fc regions in a pig relative to a control polypeptide or control polypeptides, wherein the control polypeptide or control polypeptides are identical to the polypeptide or polypeptides except for having the corresponding wild type porcine IgG Fc region in place of the IgG Fc region variant. The substitutions to increase half-life may be made in one or more of a porcine CH2 region, a porcine CH3 region, or in the context of a porcine Fc (e.g., a CH2+CH3) region.

The present disclosure provides a polypeptide comprising a porcine IgG Fc region variant, or a porcine FcRn-binding region thereof, wherein the polypeptide comprises an amino acid substitution at at least one (e.g., 1, 2, 3, 4, 5, 6, 7, 8, 9, 10, or 11) position selected from the group consisting of:
(i) a position that corresponds to amino acid position 250 of a wild type porcine IgG, wherein the amino acid substitution is selected from the group consisting of T250Q, T250E, I250Q, and I250E;
(ii) a position that corresponds to amino acid position 252 of a wild type porcine IgG, wherein the amino acid substitution is selected from the group consisting of M252Y, M252W, and M252F;
(iii) a position that corresponds to amino acid position 254 of a wild type porcine IgG, wherein the amino acid substitution is selected from the group consisting of S254T, S254R, S254K, and S254H;
(iv) a position that corresponds to amino acid position 256 of a wild type porcine IgG, wherein the amino acid substitution is selected from the group consisting of T256A, T256D, and T256E;
(v) a position that corresponds to amino acid position 286 of a wild type porcine IgG, wherein the amino acid substitution is selected from the group consisting of T286Y, T286F, T286W, T286L, T286D, and T286E;
(vi) a position that corresponds to amino acid position 309 of a wild type porcine IgG, wherein the amino acid substitution is selected from the group consisting of Q309V, Q309D, and Q309E;
(vii) a position that corresponds to amino acid position 311 of a wild type porcine IgG, wherein the amino acid substitution is selected from the group consisting of Q311V, Q311K, Q311R, Q311L, Q311H, E311V, E311K, E311R, E311L, and E311H;
(viii) a position that corresponds to amino acid position 426 of a wild type porcine IgG, wherein the amino acid substitution is selected from the group consisting of A426Y, A426F, A426H, V426Y, V426F, and V426H;
(ix) a position that corresponds to amino acid position 428 of a wild type porcine IgG, wherein the amino acid substitution is selected from the group consisting of M428L, M428Y, M428F, H428L, H428Y, and H428F;
(x) a position that corresponds to amino acid position 434 of a wild type porcine IgG, wherein the amino acid substitution is selected from the group consisting of N434A, N434H, N434F, N434S, N434W, N434Y, H434A, H434F, H434S, H434W, and H434Y; and
(xi) a position that corresponds to amino acid position 436 of a wild type porcine IgG, wherein the amino acid substitution is selected from the group consisting of Y436H and T436H.
wherein the amino acid positions are based on EU numbering, and wherein the polypeptide has increased binding affinity to bovine FcRn when compared to an Fc domain of the wild type porcine IgG.

In some embodiments, the polypeptide comprises an amino acid substitution at at least one (e.g., 1, 2, 3, 4, 5, 6, or 7) position selected from the group consisting of.
(i) a position that corresponds to amino acid position 252 of a wild type IgG of the livestock animal, wherein the amino acid substitution is M252Y;
(ii) a position that corresponds to amino acid position 286 of a wild type IgG of the livestock animal, wherein the amino acid substitution is T286E;
(iii) a position that corresponds to amino acid position 309 of a wild type IgG of the livestock animal, wherein the amino acid substitution is Q309D or Q309V;
(iv) a position that corresponds to amino acid position 311 of a wild type IgG of the livestock animal, wherein the amino acid substitution is Q311V;
(v) a position that corresponds to amino acid position 426 of a wild type IgG of the livestock animal, wherein the amino acid substitution is A426Y;
(vi) a position that corresponds to amino acid position 428 of a wild type IgG of the livestock animal, wherein the amino acid substitution is M428Y; and
(vii) a position that corresponds to amino acid position 434 of a wild type IgG of the livestock animal, wherein the amino acid substitution is selected from the group consisting of N434Y.

In some embodiments, the polypeptide has increased binding affinity to porcine FcRn at a pH of about 5.0 to about 6.5 (e.g., about 5.5 or about 6.0) when compared to an Fc domain of the wild type porcine IgG at the same pH.

In some embodiments, the polypeptide comprises an amino acid sequence that is at least 80% (e.g., at least 85%, 90%, 92%, 94%, 95%, 96%, 97%, 98%, or 99%) identical to an amino acid sequence selected from the group consisting of SEQ ID NOs: 1 to 11.

In some instances, this disclosure provides a porcine IgG CH2 domain variant comprising an amino acid sequence that is at least 75%, at least 80%, at least 85%, at least 90%, at least 95%, at least 96%, at least 97%, at least 98% or at least 99% identical to the CH2 domains shown in any one of SEQ ID NOs: 1 to 11. Also provided are porcine IgG CH2 domain variants comprising an amino acid sequence that varies from any one of the CH2 domains shown in SEQ ID NOs: 1 to 11 by 1 to 15 (e.g., by 1, 2, 3, 4, 5, 6, 7, 8, 9, 10, 11, 12, 13, 14, or 15) amino acids.

In some instances, this disclosure features a porcine IgG CH3 domain variant comprising an amino acid sequence that is at least 75%, at least 80%, at least 85%, at least 90%, at least 95%, at least 96%, at least 97% or at least 98% or at least 99%, identical to the amino acid sequence of the CH3 domain shown in any one of SEQ ID NOs: 1 to 11. Also featured are porcine IgG CH3 domain variants comprising an amino acid sequence that varies from any one of the CH3 domain sequences shown in any one of SEQ ID NOs: 1 to 11 by 1 to 15 (e.g., by 1, 2, 3, 4, 5, 6, 7, 8, 9, 10, 11, 12, 13, 14, or 15) amino acids.

In some instances, this disclosure features a porcine IgG Fc region variant comprising an amino acid sequence that is at least 75%, at least 80%, at least 85%, at least 90%, at least 95%, at least 96%, at least 97%, at least 98% or at least 99% identical to the amino acid sequence set forth in any one of SEQ ID NOs: 1 to 11. Also disclosed are porcine IgG Fc region variants comprising an amino acid sequence that varies from any one of SEQ ID NOs: 1 to 11 by 1 to 20 (e.g., by 1, 2, 3, 4, 5, 6, 7, 8, 9, 10, 11, 12, 13, 14, 15, 16, 17, 18, 19, or 20) amino acids.

In some embodiments, provided are a polypeptide or polypeptides comprising a porcine IgG Fc CH2 domain variant, the CH2 domain variant comprising an amino acid sequence that is at least 75%, at least 80%, at least 85%, at least 90%, at least 95%, at least 96%, at least 97%, at least 98% or at least 99%, identical to the amino acid sequence of the CH2 domain shown in any one of SEQ ID NOs: 1 to 11.

In some embodiments, featured are a polypeptide or polypeptides comprising a porcine IgG Fc CH3 domain variant, the CH3 domain variant comprising an amino acid sequence that is at least 75%, at least 80%, at least 85%, at least 90%, at least 95%, at least 96%, at least 97%, at least 98% or at least 99% identical to the amino acid sequence of the CH3 domain shown in any one of SEQ ID NOs: 1 to 11.

In some embodiments, featured are a polypeptide or polypeptides comprising a porcine IgG Fc region variant, the Fc region variant comprising an amino acid sequence that is at least 75%, at least 80%, at least 85%, at least 90%, at least 95%, at least 96%, at least 97%, at least 98% or at least 99% identical to the amino acid sequence set forth in any one of SEQ ID NOs: 1 to 11.

### Bovine Antibodies

Bovine typically have three IgG heavy chains referred to as IgG1, IgG2, and IgG3. These heavy chains represent three different subclasses of bovine IgG. The amino acid and DNA sequences for these heavy chains are available from the GENBANK® database. Illustrative examples of the amino acid sequences of the CH2 and CH3 domains of each of the three bovine IgG heavy chain Fc regions are shown below (CH2 domains are underlined), and the GENBANK® accession number (for the amino acid sequence or mRNA sequence from which the amino acid sequence is derived) of each heavy chain Fc region is indicated:

Bovine (Bos Taurus)
IgG1 (GenBank® ABE68619.1)
(SEQ ID NO: 12)
PPELPGGPSVFIFPPKPKDTLTISGTPEVTCVVVDVGHDDPEVKFSWFVDN
VEVNTATTKPREEQFNSTYRVVSALRIQHQDWTGGKEFKCKVHNEGLPAPI
VRTISRTKGPAREPQVYVLAPPQEELSKSTVSLTCMVTSFYPDYIAVEWQR
NGQPESEDKYGTTPPQLDADSSYFLYSKLRVDRNSWQEGDTYTCVVMHEAL
HNHYTQKSTSKSA
IgG2 (GenBank® AQT27057.1)
(SEQ ID NO: 13)
CVREPSVFIFPPKPKDTLMITGTPEVTCVVVNVGHDNPEVQFSWFVDDVEV
HTARTKPREEQFNSTYRVVSALPIQHQDWTGGKEFKCKVNIKGLSASIVRI
ISRSKGPAREPQVYVLDPPKEELSKSTVSVTCMVIGFYPEDVDVEWQRDRQ
TESEDKYRTTPPQLDADRSYFLYSKLRVDRNSWQRGGTYTCVVMHEALHNH
YMQKSTSKSAGK
IgG3 (GenBank® AAC48761.1)
(SEQ ID NO: 14)
EPLGGLSVFIFPPKPKDTLTISGTPEVTCVVVDVGQDDPEVQFSWFVDDVE
VHTARTKPREEQFNSTYRVVSALRIQHQDWLQGKEFKCKVNNKGLPAPIVR
TISRTKGQAREPQVYVLAPPREELSKSTLSLTCLITGFYPEEIDVEWQRNG
QPESEDKYHTTAPQLDADGSYFLYSKLRVNKSSWQEGDHYTCAVMHEALRN
HYKEKSISRSPGK

The CH2 region of a bovine antibody comprises or consists of amino acids 231 to 340 (according to EU numbering) of a bovine IgG antibody. It is to be understood that the CH2 region may include one to six (e.g., 1, 2, 3, 4, 5, 6) additional amino acids or deletions at their N and/or C-terminus.

The CH3 region of a bovine antibody comprises or consists of amino acids 341 to 447 (according to EU numbering) of a bovine IgG antibody. It is to be understood that the CH3 region may include one to six (e.g., 1, 2, 3, 4, 5, 6) additional amino acids or deletions at their N and/or C-terminus.

The Fc region of a bovine IgG antibody comprises or consists of amino acids 231 to 447 (according to EU numbering) of a bovine IgG antibody.

Table 2 below compares the amino acid sequences of the CH2 and CH3 domains of human IgG1, canine IgGB, and feline IgG1a with each of the three bovine IgG isotypes, based on EU numbering:

TABLE 2

| EU | Human | Canine | Feline | Bovine | Bovine | Bovine |
|---|---|---|---|---|---|---|
| number | IgG1 | IgGB | IgG1a | IgG1 | IgG2 | IgG3 |
| | CH2 Domain | | | | | |
| 231 | A | A | P | P | | |
| 232 | P | P | P | P | | |
| 233 | E | E | E | E | | E |
| 234 | L | M | M | L | | P |
| 235 | L | L | L | P | | L |
| 236 | G | G | G | G | | G |
| 237 | G | G | G | G | | G |
| 238 | P | P | P | P | P | L |
| 239 | S | S | S | S | S | S |
| 240 | V | V | I | V | V | V |
| 241 | F | F | F | F | F | F |
| 242 | L | I | I | I | I | I |
| 243 | F | F | F | F | F | F |
| 244 | P | P | P | P | P | P |
| 245 | P | P | P | P | P | P |
| 246 | K | K | K | K | K | K |
| 247 | P | P | P | P | P | P |
| 248 | K | K | K | K | K | K |
| 249 | D | D | D | D | D | D |
| 250 | T | T | T | T | T | T |
| 251 | L | L | L | L | L | L |
| 252 | M | L | S | T | M | T |
| 253 | I | I | I | I | I | I |
| 254 | S | A | S | S | T | S |
| 255 | R | R | R | G | G | G |
| 256 | T | T | T | T | T | T |
| 257 | P | P | P | P | P | P |
| 258 | E | E | E | E | E | E |
| 259 | V | V | V | V | V | V |
| 260 | T | T | T | T | T | T |
| 261 | C | C | C | C | C | C |
| 262 | V | V | L | V | V | V |
| 263 | V | V | V | V | V | V |
| 264 | V | V | V | V | V | V |
| 265 | D | D | D | D | N | D |
| 266 | V | L | L | V | V | V |
| 267 | S | D | G | G | G | G |
| 268 | H | P | P | H | H | Q |
| 269 | E | E | D | D | D | D |
| 270 | D | D | D | D | N | D |
| 271 | P | P | S | P | P | P |
| 272 | E | E | D | E | E | E |
| 273 | V | V | V | V | V | V |
| 274 | K | Q | Q | K | Q | Q |
| 275 | F | I | I | F | F | F |
| 276 | N | S | T | S | S | S |
| 277 | W | W | W | W | W | W |
| 278 | Y | F | F | F | F | F |
| 279 | V | V | V | V | V | V |
| 280 | D | D | D | D | D | D |
| 282 | G | G | N | N | D | D |
| 282 | V | K | T | V | V | V |
| 283 | E | Q | Q | E | E | E |
| 284 | V | M | V | V | V | V |
| 285 | H | Q | Y | N | H | H |
| 286 | N | T | T | T | T | T |
| 287 | A | A | A | A | A | A |
| 288 | K | K | K | T | R | R |
| 289 | T | T | T | T | T | T |
| 290 | K | Q | S | K | K | K |
| 291 | P | P | P | P | P | P |
| 292 | R | R | R | R | R | R |
| 293 | E | E | E | E | E | E |
| 294 | E | E | E | E | E | E |
| 295 | Q | Q | Q | Q | Q | Q |
| 296 | Y | F | F | F | F | F |
| 297 | N | N | N | N | N | N |
| 298 | S | G | S | S | S | S |
| 299 | T | T | T | T | T | T |
| 300 | Y | Y | Y | Y | Y | Y |
| 301 | R | R | R | R | R | R |
| 302 | V | V | V | V | V | V |
| 303 | V | V | V | V | V | V |
| 304 | S | S | S | S | S | S |
| 305 | V | V | V | A | A | A |
| 306 | L | L | L | L | L | L |
| 307 | T | P | P | R | P | R |
| 308 | V | I | I | I | I | I |
| 309 | L | G | L | Q | Q | Q |
| 310 | H | H | H | H | H | H |
| 311 | Q | Q | Q | Q | Q | Q |
| 312 | D | D | D | D | D | D |
| 313 | W | W | W | W | W | W |
| 314 | L | L | L | T | T | L |
| 315 | N | K | K | G | G | Q |
| 316 | G | G | G | G | G | G |
| 317 | K | K | K | K | K | K |
| 318 | E | Q | E | E | E | E |
| 319 | Y | F | F | F | F | F |
| 320 | K | T | K | K | K | K |
| 321 | C | C | C | C | C | C |
| 322 | K | K | K | K | K | K |
| 323 | V | V | V | V | V | V |
| 324 | S | N | N | H | N | N |
| 325 | N | N | S | N | I | N |
| 326 | K | K | K | E | K | K |
| 327 | A | A | S | G | G | G |
| 328 | L | L | L | L | L | L |
| 329 | P | P | P | P | S | P |
| 330 | A | S | S | A | A | A |
| 331 | P | P | P | P | S | P |
| 332 | I | I | I | I | I | I |
| 333 | E | E | E | V | V | V |
| 334 | K | R | R | R | R | R |
| 335 | T | T | T | T | I | T |
| 336 | I | I | I | I | I | I |
| 337 | S | S | S | S | S | S |
| 338 | K | K | K | R | R | R |
| 339 | A | A | A | T | S | T |
| 340 | K | R | K | K | K | K |
| | CH3 Domain | | | | | |
| 341 | G | G | G | G | G | G |
| 342 | Q | Q | Q | P | P | Q |
| 343 | P | A | P | A | A | A |
| 344 | R | H | H | R | R | R |
| 345 | E | Q | E | E | E | E |
| 346 | P | P | P | P | P | P |
| 347 | Q | S | Q | Q | Q | Q |
| 348 | V | V | V | V | V | V |
| 349 | Y | Y | Y | Y | Y | Y |
| 350 | T | V | V | V | V | V |
| 351 | L | L | L | L | L | L |
| 352 | P | P | P | A | D | A |
| 353 | P | P | P | P | P | P |
| 354 | S | S | A | P | P | P |
| 355 | R | R | Q | Q | K | R |
| 356 | D | E | E | E | E | E |
| 357 | E | E | E | E | E | E |
| 358 | L | L | L | L | L | L |
| 359 | T | S | S | S | S | S |
| 360 | K | K | R | K | K | K |
| 361 | N | N | N | S | S | S |
| 362 | Q | T | K | T | T | T |
| 363 | V | V | V | V | V | L |
| 364 | S | S | S | S | S | S |
| 365 | L | L | V | L | V | L |
| 366 | T | T | T | T | T | T |
| 367 | C | C | C | C | C | C |
| 368 | L | L | L | M | M | L |
| 369 | V | I | I | V | V | I |
| 370 | K | K | K | T | I | T |
| 371 | G | D | S | S | G | G |
| 372 | F | F | F | F | F | F |
| 373 | Y | F | H | Y | Y | Y |
| 374 | P | P | P | P | P | P |
| 375 | S | P | P | D | E | E |
| 376 | D | D | D | Y | D | E |
| 377 | I | I | I | I | V | I |
| 378 | A | D | A | A | D | D |
| 379 | V | V | V | V | V | V |
| 380 | E | E | E | E | E | E |
| 381 | W | W | W | W | W | W |
| 382 | E | Q | E | Q | Q | Q |
| 383 | S | S | I | R | R | R |
| 384 | N | N | T | N | D | N |
| 385 | G | G | G | G | R | G |
| 386 | Q | Q | Q | Q | Q | Q |
| 387 | P | Q | P | P | T | P |
| 388 | E | E | E | E | E | E |
| 389(a) | N | P | P | S | S | S |
| 389b | | E | E | E | E | E |
| 389c | | S | N | D | D | D |
| 390 | N | K | N | K | K | K |
| 391 | Y | Y | Y | Y | Y | Y |
| 392 | K | R | R | G | R | H |
| 393 | T | T | T | T | T | T |
| 394 | T | T | T | T | T | T |
| 395 | P | P | P | P | P | A |
| 396 | P | P | P | P | P | P |
| 397 | v | Q | Q | Q | Q | Q |
| 398 | L | L | L | L | L | L |
| 399 | D | D | D | D | D | D |
| 400 | S | E | S | A | A | A |
| 401 | D | D | D | D | D | D |
| 402 | G | G | G | S | R | G |
| 403 | S | S | T | S | S | S |
| 404 | F | Y | Y | Y | Y | Y |
| 405 | F | F | F | F | F | F |
| 406 | L | L | V | L | L | L |
| 407 | Y | Y | Y | Y | Y | Y |
| 408 | S | S | S | S | S | S |
| 409 | K | K | K | K | K | K |
| 410 | L | L | L | L | L | L |
| 411 | T | S | S | R | R | R |
| 412 | V | V | V | V | V | V |
| 413 | D | D | D | D | D | N |
| 414 | K | K | R | R | R | K |
| 415 | S | S | S | N | N | S |
| 416 | R | R | H | S | S | S |
| 417 | W | W | W | W | W | W |
| 418 | Q | Q | Q | Q | Q | Q |
| 419 | Q | R | R | E | R | E |
| 420 | G | G | G | G | G | G |
| 421 | N | D | N | D | G | D |
| 422 | V | T | T | T | T | H |
| 423 | F | F | Y | Y | Y | Y |
| 424 | S | I | T | T | T | T |
| 425 | C | C | C | C | C | C |
| 426 | S | A | S | V | V | A |
| 427 | V | V | V | V | V | V |
| 428 | M | M | S | M | M | M |
| 429 | H | H | H | H | H | H |
| 430 | E | E | E | E | E | E |
| 431 | A | A | A | A | A | A |
| 432 | L | L | L | L | L | L |
| 433 | H | H | H | H | H | R |
| 434 | N | N | S | N | N | N |
| 435 | H | H | H | H | H | H |
| 436 | Y | Y | H | Y | Y | Y |
| 437 | T | T | T | T | M | K |
| 438 | Q | Q | Q | Q | Q | E |
| 439 | K | E | K | K | K | K |
| 440 | S | S | S | S | S | S |
| 441 | L | L | L | T | T | I |
| 442 | S | S | T | S | S | S |
| 443 | L | H | Q | K | K | R |
| 444 | S | S | S | S | S | S |
| 445 | P | P | P | A | A | P |
| 446 | G | G | G | G | G | G |
| 447 | K | K | K | K | K | K |

Substitutions in Bovine IgG Fc that Improve Half-Life

Increased serum persistence is a beneficial property for therapeutic polypeptides. This disclosure features substitutions in wild type bovine IgG1, IgG2, and IgG3 Fc regions that enhance the half-life of a polypeptide or polypeptides comprising these Fc regions in a bovine relative to a control polypeptide or control polypeptides, wherein the control polypeptide or control polypeptides are identical to the polypeptide or polypeptides except for having the corresponding wild type bovine IgG Fc region in place of the IgG Fc region variant. The substitutions to increase half-life may be made in one or more of a bovine CH2 domain, a bovine CH3 domain, or in the context of a bovine Fc (e.g., a CH2+CH3) region.

The present disclosure provides a polypeptide comprising a bovine IgG Fc region variant, or a bovine FcRn-binding region thereof, wherein the polypeptide comprises an amino acid substitution at at least one (e.g., 1, 2, 3, 4, 5, 6, 7, 8, 9, 10, or 11) position selected from the group consisting of:
(i) a position that corresponds to amino acid position 250 of a wild type bovine IgG, wherein the amino acid substitution is selected from the group consisting of T250Q and T250E;
(ii) a position that corresponds to amino acid position 252 of a wild type bovine IgG, wherein the amino acid substitution is selected from the group consisting of M252Y, M252W, M252F, T252Y, T252W, and T252F;
(iii) a position that corresponds to amino acid position 254 of a wild type bovine IgG, wherein the amino acid substitution is selected from the group consisting of S254T, S254R, S254K, S254H, T254R, T254K, and T254H;
(iv) a position that corresponds to amino acid position 256 of a wild type bovine IgG, wherein the amino acid substitution is selected from the group consisting of T256A, T256D, and T256E;
(v) a position that corresponds to amino acid position 286 of a wild type bovine IgG, wherein the amino acid substitution is selected from the group consisting of T286Y, T286F, T286W, T286L, T286D, and T286E;
(vi) a position that corresponds to amino acid position 309 of a wild type bovine IgG, wherein the amino acid substitution is selected from the group consisting of Q309V, Q309D, and Q309E;
(vii) a position that corresponds to amino acid position 311 of a wild type bovine IgG, wherein the amino acid substitution is selected from the group consisting of Q311V, Q311K, Q311R, Q311L, and Q311H;
(viii) a position that corresponds to amino acid position 426 of a wild type bovine IgG, wherein the amino acid substitution is selected from the group consisting of A426Y, A426F, A426H, V426Y, V426F, and V426H;
(ix) a position that corresponds to amino acid position 434 of a wild type bovine IgG, wherein the amino acid substitution is selected from the group consisting of N434A, N434H, N434F, N434S, N434W, and N434Y; and
(x) a position that corresponds to amino acid position 436 of a wild type bovine IgG, wherein the amino acid substitution is Y436H, wherein the amino acid positions are based on EU numbering, and wherein the polypeptide has increased binding affinity to bovine FcRn when compared to an Fc domain of the wild type bovine IgG.

In some embodiments, the polypeptide comprises an amino acid substitution at at least one (e.g., 1, 2, 3, 4, or 5) position selected from the group consisting of:
(i) a position that corresponds to amino acid position 252 of a wild type IgG of the livestock animal, wherein the amino acid substitution is T252Y;
(ii) a position that corresponds to amino acid position 309 of a wild type IgG of the livestock animal, wherein the amino acid substitution is Q309V;
(iii) a position that corresponds to amino acid position 311 of a wild type IgG of the livestock animal, wherein the amino acid substitution is Q311V;
(iv) a position that corresponds to amino acid position 434 of a wild type IgG of the livestock animal, wherein the amino acid substitution is selected from the group consisting of N434Y; and
(v) a position that corresponds to amino acid position 436 of a wild type IgG of the livestock animal, wherein the amino acid substitution is selected from the group consisting of Y436H.

In some embodiments, the polypeptide has increased binding affinity to bovine FcRn at a pH of about 5.0 to about 6.5 (e.g., about 5.5 or about 6.0) when compared to an Fc domain of the wild type bovine IgG at the same pH.

In some embodiments, the polypeptide comprises an amino acid sequence that is at least 80% (e.g., at least 85%, 90%, 92%, 94%, 95%, 96%, 97%, 98%, or 99%) identical to an amino acid sequence selected from the group consisting of SEQ ID NOs: 12 to 14.

In some instances, this disclosure provides a bovine IgG CH2 domain variant comprising an amino acid sequence that is at least 75%, at least 80%, at least 85%, at least 90%, at least 95%, at least 96%, at least 97%, at least 98% or at least 99% identical to the CH2 domains shown in any one of SEQ ID NOs: 12 to 14. Also provided are bovine IgG CH2 domain variants comprising an amino acid sequence that varies from any one of the CH2 domains shown in SEQ ID NOs: 12 to 14 by 1 to 15 (e.g., by 1, 2, 3, 4, 5, 6, 7, 8, 9, 10, 11, 12, 13, 14, or 15) amino acids.

In other instances, this disclosure features a bovine IgG CH3 domain variant comprising an amino acid sequence that is at least 75%, at least 80%, at least 85%, at least 90%, at least 95%, at least 96%, at least 97% or at least 98% or at least 99%, identical to the amino acid sequence of the CH3 domain shown in any one of SEQ ID NOs: 12 to 14. Also featured are bovine IgG CH3 domain variants comprising an amino acid sequence that varies from any one of the CH3 domain sequences shown in any one of SEQ ID NOs: 12 to 14 by 1 to 15 (e.g., by 1, 2, 3, 4, 5, 6, 7, 8, 9, 10, 11, 12, 13, 14, or 15) amino acids.

In certain instances, this disclosure features a bovine IgG Fc region variant comprising an amino acid sequence that is at least 75%, at least 80%, at least 85%, at least 90%, at least 95%, at least 96%, at least 97%, at least 98% or at least 99% identical to the amino acid sequence set forth in any one of SEQ ID NOs: 12 to 14. Also disclosed are bovine IgG Fc region variants comprising an amino acid sequence that varies from any one of SEQ ID NOs: 12 to 14 by 1 to 20 (e.g., by 1, 2, 3, 4, 5, 6, 7, 8, 9, 10, 11, 12, 13, 14, 15, 16, 17, 18, 19, or 20) amino acids.

In some embodiments, provided are a polypeptide or polypeptides comprising a bovine IgG Fc CH2 domain variant, the CH2 domain variant comprising an amino acid sequence that is at least 75%, at least 80%, at least 85%, at least 90%, at least 95%, at least 96%, at least 97%, at least 98% or at least 99%, identical to the amino acid sequence of the CH2 domain shown in any one of SEQ ID NOs: 12 to 14.

In some embodiments, featured are a polypeptide or polypeptides comprising a bovine IgG Fc CH3 domain variant, the CH3 domain variant comprising an amino acid sequence that is at least 75%, at least 80%, at least 85%, at least 90%, at least 95%, at least 96%, at least 97%, at least 98% or at least 99% identical to the amino acid sequence of the CH3 domain shown in any one of SEQ ID NOs: 12 to 14.

In some embodiments, featured are a polypeptide or polypeptides comprising a bovine IgG Fc region variant, the Fc region variant comprising an amino acid sequence that is at least 75%, at least 80%, at least 85%, at least 90%, at least 95%, at least 96%, at least 97%, at least 98% or at least 99% identical to the amino acid sequence set forth in any one of SEQ ID NOs: 12 to 14.

### Equine Antibodies

Equine typically have seven IgG heavy chains referred to as IgG1, IgG2, IgG3, IgG4, IgG5, IgG6, and IgG7. These heavy chains represent seven different subclasses of equine IgG. The amino acid and DNA sequences for these heavy chains are available from the GENBANK® database. Illustrative examples of the amino acid sequences of the CH2 and CH3 domains of each of the seven equine IgG heavy chain Fc regions are shown below (CH2 domains are underlined), and the GENBANK® accession number (for the amino acid sequence or mRNA sequence from which the amino acid sequence is derived) of each heavy chain Fc region is indicated:

Horse (Equus caballus)
IgG1 (GenBank® CAC44760.1)
(SEQ ID NO: 15)
APELLGGPSVFIFPPNPKDTLMITRTPEVTCVVVDVSQENPDVKFNWYMDG
VEVRTATTRPKEEQFNSTYRVVSVLRIQHQDWLSGKEFKCKVNNQALPQPI
ERTITKTKGRSQEPQVYVLAPHPDELSKSKVSVTCLVKDFYPPEINIEWQS
NGQPELETKYSTTQAQQDSDGSYFLYSKLSVDRNRWQQGTTFTCGVMHEAL
HNHYTQKNVSKNPGK
IgG2 (GenBank® CAC44761.1)
(SEQ ID NO: 16)
GPSVFIFPPNPKDALMISRTPVVTCVVVNLSDQYPDVQFSWYVDNTEVHSA
ITKQREAQFNSTYRVVSVLPIQHQDWLSGKEFKCSVTNVGVPQPISRAISR
GKGPSRVPQVYVLPPHPDELAKSKVSVTCLVKDFYPPDISVEWQSNRWPEL
EGKYSTTPAQLDGDGSYFLYSKLSLETSRWQQVESFTCAVMHEALHNHFTK
TDISESLGK
IgG3 (GenBank® AAP82181.1)
(SEQ ID NO: 17)
APELLGGPSVFIFPPKPKDVLMITRMPEVTCLVVDVSHDSSDVLFTWYVDG
TEVKTAKTMPNEEQNNSTYRVVSVLRIQHQDWLNGKKFKCKVNNQALPAPV
ERTISKATGQTRVPQVYVLAPHPDELSKNKVSVTCLVKDFYPTDITVEWQS
NEHPEPEGKYRTTEAQKDSDGSYFLYSKLTVEKDRWQQGTTFTCVVMHEAL
HNHVMQKNISKNPGK
IgG4 (GenBank® AA518415.1)
(SEQ ID NO: 18)
ECLQVGPSVFIFPPKPKDVLMISRTPTVTCVVVDVGHDFPDVQFNWYVDGV
ETHTATTEPKQEQFNSTYRVVSVLPIQHKDWLSGKEFKCKVNNKALPAPVE
RTISKPTGQPREPQVYVLAPHRDELSKNKVSVTCLVKDFYPTDIDIEWKSN
GQPEPETKYSTTPAQLDSDGSYFLYSKLTVETNRWQQGTTFTCAVMHEALH
NHYTEKSVSKSPGK
IgG5 (GenBank® CAC86340.1)
(SEQ ID NO: 19)
APELPGGPSVFIFPPKPKDVLKISRKPEVTCVVVDLGHDDPDVQFTWFVDG
VETHTATTEPKEEQFNSTYRVVSVLPIQHQDWLSGKEFKCSVTNKALPAPV
ERTTSKAKGQLRVPQVYVLAPHPDELAKNTVSVTCLVKDFYPPEIDVEWQS
NEHPEPEGKYSTTPAQLNSDGSYFLYSKLSVETSRWKQGESFTCGVMHEAV
ENHYTQKNVSHSPGK
IgG6 (GenBank® CAC86341.1)
(SEQ ID NO: 20)
DSKFLGRPSVFIFPPNPKDTLMISRTPEVTCVVVDVSQENPDVKFNWYVDG
VEAHTATTKAKEKQDNSTYRVVSVLPIQHQDWRRGKEFKCKVNNRALPAPV
ERTITKAKGELQDPKVYILAPHREEVTKNTVSVTCLVKDFYPPDINVEWQS
NEEPEPEVKYSTTPAQLDGDGSYFLYSKLTVETDRWEQGESFTCVVMHEAI
RHTYRQKSITNFPGK
IgG7 (GenBank® AAS18414.1)
(SEQ ID NO: 21)
ECLSVGPSVFIFPPKPKDVLMISRTPTVTCVVVDVGHDFPDVQFNWYVDGV
ETHTATTEPKQEQNNSTYRVVSILAIQHKDWLSGKEFKCKVNNQALPAPVQ
KTISKPTGQPREPQVYVLAPHRDELSKNKVSVTCLVKDFYPTDIDIEWKSN
GQPEPETKYSTTPAQLDSDGSYFLYSKLTVETNRWQQGTTFTCAVMHEALH
NHYTEKSVSKSPGK

The CH2 region of an equine antibody comprises or consists of amino acids 231 to 340 (according to EU numbering) of an equine IgG antibody. It is to be understood that the CH2 region may include one to six (e.g., 1, 2, 3, 4, 5, 6) additional amino acids or deletions at their N and/or C-terminus.

The CH3 region of an equine antibody comprises or consists of amino acids 341 to 447 (according to EU numbering) of an equine IgG antibody. It is to be understood that the CH3 region may include one to six (e.g., 1, 2, 3, 4, 5, 6) additional amino acids or deletions at their N and/or C-terminus.

The Fc region of an equine IgG antibody comprises or consists of amino acids 231 to 447 (according to EU numbering) of an equine IgG antibody.

Table 3 below compares the amino acid sequences of the CH2 and CH3 domains of human IgG1, canine IgGB, and feline IgG1a with each of the seven equine IgG isotypes, based on EU numbering:

TABLE 3

| | | | | | | | | | | |
|---|---|---|---|---|---|---|---|---|---|---|
| EU | Human | Canine | Feline | Horse | Horse | Horse | Horse | Horse | Horse | Horse |
| number | IgG1 | IgGB | IgG1a | IgG1 | IgG2 | IgG3 | IgG4 | IgG5 | IgG6 | IgG7 |

CH2 Domain

| 231 | A | A | P | A | | A | | A | D | |
|---|---|---|---|---|---|---|---|---|---|---|
| 232 | P | P | P | P | | P | E | P | S | E |
| 233 | E | E | E | E | | E | C | E | K | C |
| 234 | L | M | M | L | | L | L | L | F | L |
| 235 | L | L | L | L | | L | Q | P | L | S |
| 236 | G | G | G | G | | G | V | G | G | V |
| 237 | G | G | G | G | G | G | G | G | R | G |
| 238 | P | P | P | P | P | P | P | P | P | P |
| 239 | S | S | S | S | S | S | S | S | S | S |
| 240 | V | V | I | V | V | V | V | V | V | V |
| 241 | F | F | F | F | F | F | F | F | F | F |
| 242 | L | I | I | I | I | I | I | I | I | I |
| 243 | F | F | F | F | F | F | F | F | F | F |
| 244 | P | P | P | P | P | P | P | P | P | P |
| 245 | P | P | P | P | P | P | P | P | P | P |
| 246 | K | K | K | N | N | K | K | K | N | K |
| 247 | P | P | P | P | P | P | P | P | P | P |
| 248 | K | K | K | K | K | K | K | K | K | K |
| 249 | D | D | D | D | D | D | D | D | D | D |
| 250 | T | T | T | T | A | V | V | V | T | V |
| 251 | L | L | L | L | L | L | L | L | L | L |
| 252 | M | L | S | M | M | M | M | K | M | M |
| 253 | I | I | I | I | I | I | I | I | I | I |
| 254 | S | A | S | T | S | T | S | S | S | S |
| 255 | R | R | R | R | R | R | R | R | R | R |
| 256 | T | T | T | T | T | M | T | K | T | T |
| 257 | P | P | P | P | P | P | P | P | P | P |
| 258 | E | E | E | E | V | E | T | E | E | T |
| 259 | V | V | V | V | V | V | V | V | V | V |
| 260 | T | T | T | T | T | T | T | T | T | T |
| 261 | C | C | C | C | C | C | C | C | C | C |
| 262 | V | V | L | V | V | L | V | V | V | V |
| 263 | V | V | V | V | V | V | V | V | V | V |
| 264 | V | V | V | V | V | V | V | V | V | V |
| 265 | D | D | D | D | N | D | D | D | D | D |
| 266 | V | L | L | V | L | V | V | L | V | V |
| 267 | S | D | G | S | S | S | G | G | S | G |
| 268 | H | P | P | Q | D | H | H | H | Q | H |
| 269 | E | E | D | E | Q | D | D | D | E | D |
| 270 | D | D | D | N | Y | S | F | D | N | F |
| 271 | P | P | S | P | P | S | P | P | P | P |
| 272 | E | E | D | D | D | D | D | D | D | D |
| 273 | V | V | V | V | V | V | V | V | V | V |
| 274 | K | Q | Q | K | Q | L | Q | Q | K | Q |
| 275 | F | I | I | F | F | F | F | F | F | F |
| 276 | N | S | T | N | S | T | N | T | N | N |
| 277 | W | W | W | W | W | W | W | W | W | W |
| 278 | Y | F | F | Y | Y | Y | Y | F | Y | Y |
| 279 | V | V | V | M | V | V | V | V | V | V |
| 280 | D | D | D | D | D | D | D | D | D | D |
| 282 | G | G | N | G | N | G | G | G | G | G |
| 282 | V | K | T | V | T | T | V | V | V | V |
| 283 | E | Q | Q | E | E | E | E | E | E | E |
| 284 | V | M | V | V | V | V | T | T | A | T |
| 285 | H | Q | Y | R | H | K | H | H | H | H |
| 286 | N | T | T | T | S | T | T | T | T | T |
| 287 | A | A | A | A | A | A | A | A | A | A |
| 288 | K | K | K | T | I | K | T | T | T | T |
| 289 | T | T | T | T | T | T | T | T | T | T |
| 290 | K | Q | S | R | K | M | E | E | K | E |
| 291 | P | P | P | P | Q | P | P | P | A | P |
| 292 | R | R | R | K | R | N | K | K | K | K |
| 293 | E | E | E | E | E | E | Q | E | E | Q |
| 294 | E | E | E | E | A | E | E | E | K | E |
| 295 | Q | Q | Q | Q | Q | Q | Q | Q | Q | Q |
| 296 | Y | F | F | F | F | N | F | F | D | N |
| 297 | N | N | N | N | N | N | N | N | N | N |
| 298 | S | G | S | S | S | S | S | S | S | S |
| 299 | T | T | T | T | T | T | T | T | T | T |
| 300 | Y | Y | Y | Y | Y | Y | Y | Y | Y | Y |
| 301 | R | R | R | R | R | R | R | R | R | R |
| 302 | V | V | V | V | V | V | V | V | V | V |
| 303 | V | V | V | V | V | V | V | V | V | V |
| 304 | S | S | S | S | S | S | S | S | S | S |
| 305 | V | V | V | V | V | V | V | V | V | I |
| 306 | L | L | L | L | L | L | L | L | L | L |
| 307 | T | P | P | R | P | R | P | P | P | A |
| 308 | V | I | I | I | I | I | I | I | I | I |
| 309 | L | G | L | Q | Q | Q | Q | Q | Q | Q |
| 310 | H | H | H | H | H | H | H | H | H | H |
| 311 | Q | Q | Q | Q | Q | Q | K | Q | Q | K |
| 312 | D | D | D | D | D | D | D | D | D | D |
| 313 | W | W | W | W | W | W | W | W | W | W |
| 314 | L | L | L | L | L | L | L | L | R | L |
| 315 | N | K | K | S | S | N | S | S | R | S |
| 316 | G | G | G | G | G | G | G | G | G | G |
| 317 | K | K | K | K | K | K | K | K | K | K |
| 318 | E | Q | E | E | E | K | E | E | E | E |
| 319 | Y | F | F | F | F | F | F | F | F | F |
| 320 | K | T | K | K | K | K | K | K | K | K |
| 321 | C | C | C | C | C | C | C | C | C | C |
| 322 | K | K | K | K | S | K | K | S | K | K |
| 323 | V | V | V | V | V | V | V | V | V | V |
| 324 | S | N | N | N | T | N | N | T | N | N |
| 325 | N | N | S | N | N | N | N | N | N | N |
| 326 | K | K | K | Q | v | Q | K | K | R | Q |
| 327 | A | A | S | A | G | A | A | A | A | A |
| 328 | L | L | L | L | V | L | L | L | L | L |
| 329 | P | P | P | P | P | P | P | P | P | P |
| 330 | A | S | S | Q | Q | A | A | A | A | A |
| 331 | P | P | P | P | P | P | P | P | P | P |
| 332 | I | I | I | I | I | V | V | V | V | V |
| 333 | E | E | E | E | S | E | E | E | E | Q |
| 334 | K | R | R | R | R | R | R | R | R | K |
| 335 | T | T | T | T | A | T | T | T | T | T |
| 336 | I | I | I | I | I | I | I | T | I | I |
| 337 | 5 | 5 | 5 | T | S | S | S | S | T | S |
| 338 | K | K | K | K | R | K | K | K | K | K |
| 339 | A | A | A | T | G | A | P | A | A | P |
| 340 | K | R | K | K | K | T | T | K | K | T |
| | CH3 Domain | | | | | | | | | |
| 341 | G | G | G | G | G | G | G | G | G | G |
| 342 | Q | Q | Q | R | P | Q | Q | Q | E | Q |
| 343 | P | A | P | S | S | T | P | L | L | P |
| 344 | R | H | H | Q | R | R | R | R | Q | R |
| 345 | E | Q | E | E | V | V | E | V | D | E |
| 346 | P | P | P | P | P | P | P | P | P | P |
| 347 | Q | s | Q | Q | Q | Q | Q | Q | K | Q |
| 348 | V | V | V | V | V | V | V | V | V | V |
| 349 | Y | Y | Y | Y | Y | Y | Y | Y | Y | Y |
| 350 | T | V | V | V | V | V | V | V | I | V |
| 351 | L | L | L | L | L | L | L | L | L | L |
| 352 | P | P | P | A | P | A | A | A | A | A |
| 353 | P | P | P | P | P | P | P | P | P | P |
| 354 | S | S | A | H | H | H | H | H | H | H |
| 355 | R | R | Q | P | P | P | R | P | R | R |
| 356 | D | E | E | D | D | D | D | D | E | D |
| 357 | E | E | E | E | E | E | E | E | E | E |
| 358 | L | L | L | L | L | L | L | L | V | L |
| 359 | T | S | S | S | A | S | S | A | T | S |
| 360 | K | K | R | K | K | K | K | K | K | K |
| 361 | N | N | N | S | S | N | N | N | N | N |
| 362 | Q | T | K | K | K | K | K | T | T | K |
| 363 | V | V | V | V | V | V | V | V | V | V |
| 364 | S | S | S | S | S | S | S | S | S | S |
| 365 | L | L | V | V | V | V | V | V | V | V |
| 366 | T | T | T | T | T | T | T | T | T | T |
| 367 | C | C | C | C | C | C | C | C | C | C |
| 368 | L | L | L | L | L | L | L | L | L | L |
| 369 | V | I | I | V | V | V | V | V | V | V |
| 370 | K | K | K | K | K | K | K | K | K | K |
| 371 | G | D | S | D | D | D | D | D | D | D |
| 372 | F | F | F | F | F | F | F | F | F | F |
| 373 | Y | F | H | Y | Y | Y | Y | Y | Y | Y |
| 374 | P | P | P | P | P | P | P | P | P | P |
| 375 | S | P | P | P | P | T | T | P | P | T |
| 376 | D | D | D | E | D | D | D | E | D | D |
| 377 | I | I | I | I | I | I | I | I | I | I |
| 378 | A | D | A | N | S | T | D | D | N | D |
| 379 | V | V | V | I | V | V | I | V | V | I |
| 380 | E | E | E | E | E | E | E | E | E | E |
| 381 | W | W | W | W | W | W | W | W | W | W |
| 382 | E | Q | E | Q | Q | Q | K | Q | Q | K |
| 383 | S | S | I | S | S | S | S | S | S | S |
| 384 | N | N | T | N | N | N | N | N | N | N |
| 385 | G | G | G | G | R | E | G | E | E | G |
| 386 | Q | Q | Q | Q | W | H | Q | H | E | Q |
| 387 | P | Q | P | P | P | P | P | P | P | P |
| 388 | E | E | E | E | E | E | E | E | E | E |
| 389(a) | N | P | P | L | L | P | P | P | P | P |
| 389h | | E | E | E | E | E | E | E | E | E |
| 389c | | S | N | T | G | G | T | G | V | T |
| 390 | N | K | N | K | K | K | K | K | K | K |
| 391 | Y | Y | Y | Y | Y | Y | Y | Y | Y | Y |
| 392 | K | R | R | S | S | R | S | S | S | S |
| 393 | T | T | T | T | T | T | T | T | T | T |
| 394 | T | T | T | T | T | T | T | T | T | T |
| 395 | P | P | P | Q | P | E | P | P | P | P |
| 396 | P | P | P | A | A | A | A | A | A | A |
| 397 | V | Q | Q | Q | Q | Q | Q | Q | Q | Q |
| 398 | L | L | L | Q | L | K | L | L | L | L |
| 399 | D | D | D | D | D | D | D | N | D | D |
| 400 | S | E | S | S | G | S | S | S | G | S |
| 401 | D | D | D | D | D | D | D | D | D | D |
| 402 | G | G | G | G | G | G | G | G | G | G |
| 403 | S | S | T | S | S | S | S | S | S | S |
| 404 | F | Y | Y | Y | Y | Y | Y | Y | Y | Y |
| 405 | F | F | F | F | F | F | F | F | F | F |
| 406 | L | L | V | L | L | L | L | L | L | L |
| 407 | Y | Y | Y | Y | Y | Y | Y | Y | Y | Y |
| 408 | S | S | S | S | S | S | S | S | S | S |
| 409 | K | K | K | K | K | K | K | K | K | K |
| 410 | L | L | L | L | L | L | L | L | L | L |
| 411 | T | S | S | S | S | T | T | S | T | T |
| 412 | V | V | V | V | L | V | V | V | V | V |
| 413 | D | D | D | D | E | E | E | E | E | E |
| 414 | K | K | R | R | T | K | T | T | T | T |
| 415 | S | S | S | N | S | D | N | S | D | N |
| 416 | R | R | H | R | R | R | R | R | R | R |
| 417 | W | W | W | W | W | W | W | W | W | W |
| 418 | Q | Q | Q | Q | Q | Q | Q | K | E | Q |
| 419 | Q | R | R | Q | Q | Q | Q | Q | Q | Q |
| 420 | G | G | G | G | V | G | G | G | G | G |
| 421 | N | D | N | T | E | T | T | E | E | T |
| 422 | V | T | T | T | S | T | T | S | S | T |
| 423 | F | F | Y | F | F | F | F | F | F | F |
| 424 | S | I | T | T | T | T | T | T | T | T |
| 425 | C | C | C | C | C | C | C | C | C | C |
| 426 | S | A | S | G | A | V | A | G | V | A |
| 427 | V | V | V | V | V | V | V | V | V | V |
| 428 | M | M | S | M | M | M | M | M | M | M |
| 429 | H | H | H | H | H | H | H | H | H | H |
| 430 | E | E | E | E | E | E | E | E | E | E |
| 431 | A | A | A | A | A | A | A | A | A | A |
| 432 | L | L | L | L | L | L | L | V | I | L |
| 433 | H | H | H | H | H | H | H | E | R | H |
| 434 | N | N | S | N | N | N | N | N | H | N |
| 435 | H | H | H | H | H | H | H | H | T | H |
| 436 | Y | Y | H | Y | F | V | Y | Y | Y | Y |
| 437 | T | T | T | T | T | M | T | T | R | T |
| 438 | Q | Q | Q | Q | K | Q | E | Q | Q | E |
| 439 | K | E | K | K | T | K | K | K | K | K |
| 440 | S | S | S | N | D | N | S | N | S | S |
| 441 | L | L | L | V | I | I | V | V | I | V |
| 442 | S | S | T | S | S | S | S | S | T | S |
| 443 | L | H | Q | K | E | K | K | H | N | K |
| 444 | S | S | S | N | S | N | S | S | F | S |
| 445 | P | P | P | P | L | P | P | P | P | P |
| 446 | G | G | G | G | G | G | G | G | G | G |
| 447 | K | K | K | K | K | K | K | K | K | K |

Substitutions in Equine IgG Fc that Improve Half-Life

Increased serum persistence is a beneficial property for therapeutic polypeptides. This disclosure features substitutions in wild type equine IgG1, IgG2, IgG3, IgG4, IgG5, IgG6, and IgG7 Fc regions that enhance the half-life of a polypeptide or polypeptides comprising these Fc regions in an equine relative to a control polypeptide or control polypeptides, wherein the control polypeptide or control polypeptides are identical to the polypeptide or polypeptides except for having the corresponding wild type equine IgG Fc region in place of the IgG Fc region variant. The substitutions to increase half-life may be made in one or more of an equine CH2 domain, an equine CH3 domain, or in the context of an equine Fc (e.g., a CH2+CH3) region.

The present disclosure provides a polypeptide comprising an equine IgG Fc region variant, or an equine FcRn-binding region thereof, wherein the polypeptide comprises an amino acid substitution at at least one (e.g., 1, 2, 3, 4, 5, 6, 7, 8, 9, 10, or 11) position selected from the group consisting of:
(i) a position that corresponds to amino acid position 250 of a wild type equine IgG, wherein the amino acid substitution is selected from the group consisting of T250Q, T250E, A250Q, A250E, V250Q, and V250E;
(ii) a position that corresponds to amino acid position 252 of a wild type equine IgG, wherein the amino acid substitution is selected from the group consisting of M252Y, M252W, M252F, K252Y, K252W, and K252F;
(iii) a position that corresponds to amino acid position 254 of a wild type equine IgG, wherein the amino acid substitution is selected from the group consisting of S254T, S254R, S254K, S254H, T254R, T254K, and T254H;
(iv) a position that corresponds to amino acid position 256 of a wild type equine IgG, wherein the amino acid substitution is selected from the group consisting of T256A, T256D, T256E, M256A, M256D, M256E, K256A, K256D, and K256E;
(v) a position that corresponds to amino acid position 286 of a wild type equine IgG, wherein the amino acid substitution is selected from the group consisting of S286Y, S286F, S286W, S286L, S286D, S286E, T286Y, T286F, T286W, T286L, T286D, and T286E;
(vi) a position that corresponds to amino acid position 309 of a wild type equine IgG, wherein the amino acid substitution is selected from the group consisting of Q309V, Q309D, and Q309E;
(vii) a position that corresponds to amino acid position 311 of a wild type equine IgG, wherein the amino acid substitution is selected from the group consisting of Q311V, Q311K, Q311R, Q311L, Q311H, K311V, K311R, K311L, and K311H;
(viii) a position that corresponds to amino acid position 426 of a wild type equine IgG, wherein the amino acid substitution is selected from the group consisting of A426Y, A426F, A426H, V426Y, V426F, V426H, G426Y, G426F, and G426H;
(ix) a position that corresponds to amino acid position 428 of a wild type equine IgG, wherein the amino acid substitution is selected from the group consisting of M428L, M428Y, and M428F;
(x) a position that corresponds to amino acid position 434 of a wild type equine IgG, wherein the amino acid substitution is selected from the group consisting of N434A, N434H, N434F, N434S, N434W, N434Y, H434A, H434F, H434S, H434W, and H434Y; and
(xi) a position that corresponds to amino acid position 436 of a wild type equine IgG, wherein the amino acid substitution is selected from the group consisting of Y436H, F436H, and V436H,
wherein the amino acid positions are based on EU numbering, and wherein the polypeptide has increased binding affinity to equine FcRn when compared to an Fc domain of the wild type equine IgG.

In some embodiments, the polypeptide comprises an amino acid substitution at at least one (e.g., 1, 2, 3, 4, 5, or 6) position selected from the group consisting of.
(i) a position that corresponds to amino acid position 252 of a wild type IgG of the livestock animal, wherein the amino acid substitution is M252Y;
(ii) a position that corresponds to amino acid position 286 of a wild type IgG of the livestock animal, wherein the amino acid substitution is T286E
(iii) a position that corresponds to amino acid position 309 of a wild type IgG of the livestock animal, wherein the amino acid substitution is Q309V;
(iv) a position that corresponds to amino acid position 311 of a wild type IgG of the livestock animal, wherein the amino acid substitution is Q311V;
(v) a position that corresponds to amino acid position 426 of a wild type IgG of the livestock animal, wherein the amino acid substitution is G426Y; and
(vi) a position that corresponds to amino acid position 434 of a wild type IgG of the livestock animal, wherein the amino acid substitution is selected from the group consisting of N434Y.

In some embodiments, the polypeptide has increased binding affinity to equine FcRn at a pH of about 5.0 to about 6.5 (e.g., about 5.5 or about 6.0) when compared to an Fc domain of the wild type equine IgG at the same pH.

In some embodiments, the polypeptide comprises an amino acid sequence that is at least 80% (e.g., at least 85%, 90%, 92%, 94%, 95%, 96%, 97%, 98%, or 99%) identical to an amino acid sequence selected from the group consisting of SEQ ID NOs: 15 to 21.

In some instances, this disclosure provides an equine IgG CH2 domain variant comprising an amino acid sequence that is at least 75%, at least 80%, at least 85%, at least 90%, at least 95%, at least 96%, at least 97%, at least 98% or at least 99% identical to the CH2 domains shown in any one of SEQ ID NOs: 15 to 21. Also provided are equine IgG CH2 domain variants comprising an amino acid sequence that varies from any one of the CH2 domains shown in SEQ ID NOs: 15 to 21 by 1 to 15 (e.g., by 1, 2, 3, 4, 5, 6, 7, 8, 9, 10, 11, 12, 13, 14, or 15) amino acids.

In some instances, this disclosure features an equine IgG CH3 domain variant comprising an amino acid sequence that is at least 75%, at least 80%, at least 85%, at least 90%, at least 95%, at least 96%, at least 97% or at least 98% or at least 99%, identical to the amino acid sequence of the CH3 domain shown in any one of SEQ ID NOs: 15 to 21. Also featured are equine IgG CH3 domain variants comprising an amino acid sequence that varies from any one of the CH3 domain sequences shown in any one of SEQ ID NOs: 15 to 21 by 1 to 15 (e.g., by 1, 2, 3, 4, 5, 6, 7, 8, 9, 10, 11, 12, 13, 14, or 15) amino acids.

In some instances, this disclosure features an equine IgG Fc region variant comprising an amino acid sequence that is at least 75%, at least 80%, at least 85%, at least 90%, at least 95%, at least 96%, at least 97%, at least 98% or at least 99% identical to the amino acid sequence set forth in any one of SEQ ID NOs: 15 to 21. Also disclosed are equine IgG Fc region variants comprising an amino acid sequence that varies from any one of SEQ ID NOs: 15 to 21 by 1 to 20 (e.g., by 1, 2, 3, 4, 5, 6, 7, 8, 9, 10, 11, 12, 13, 14, 15, 16, 17, 18, 19, or 20) amino acids.

In some embodiments, provided are a polypeptide or polypeptides comprising an equine IgG Fc CH2 domain variant, the CH2 domain variant comprising an amino acid sequence that is at least 75%, at least 80%, at least 85%, at least 90%, at least 95%, at least 96%, at least 97%, at least 98% or at least 99%, identical to the amino acid sequence of the CH2 domain shown in any one of SEQ ID NOs: 15 to 21.

In some embodiments, featured are a polypeptide or polypeptides comprising an equine IgG Fc CH3 domain variant, the CH3 domain variant comprising an amino acid sequence that is at least 75%, at least 80%, at least 85%, at least 90%, at least 95%, at least 96%, at least 97%, at least 98% or at least 99% identical to the amino acid sequence of the CH3 domain shown in any one of SEQ ID NOs: 15 to 21.

In some embodiments, featured are a polypeptide or polypeptides comprising an equine IgG Fc region variant, the Fc region variant comprising an amino acid sequence that is at least 75%, at least 80%, at least 85%, at least 90%, at least 95%, at least 96%, at least 97%, at least 98% or at least 99% identical to the amino acid sequence set forth in any one of SEQ ID NOs: 15 to 21.

### Ovine Antibodies

Ovine typically have three IgG heavy chains referred to as IgG1, IgG2, and IgG3. These heavy chains represent three different subclasses of ovine IgG. The amino acid and DNA sequences for these heavy chains are available from the GENBANK® database. Illustrative examples of the amino acid sequences of the CH2 and CH3 domains of each of the three ovine IgG heavy chain Fc regions are shown below (CH2 domains are underlined), and the GENBANK® accession number (for the amino acid sequence or mRNA sequence from which the amino acid sequence is derived) of each heavy chain Fc region is indicated:

Sheep (Ovis aries)
IgG1 (GENBANK ® X69797)
(SEQ ID NO: 22)
PELPGGPSVFIFPPKPKDTLTISGTPEVTCVVVDVGQDDPEVQFSWFVDN
VEVRTARTKPREEQFNSTFRVVSALPIQHQDWTGGKEFKCKVHNEALPAP
IVRTISRTKGQAREPQVYVLAPPQEELSKSTLSVTCLVTGFYPDYIAVEW
QKNGQPESEDKYGTTTSQLDADGSYFLYSRLRVDKNSWQEGDTYACVVMH
EALHNHYTQKSISKPPGK
IgG2 (GENBANK ® X70983)
(SEQ ID NO: 23)
VSRPSVFIFPPKPKDSLMITGTPEVTCVVVDVGQGDPEVQFSWFVDNVEV
RTARTKPREEQFNSTFRVVSALPIQHDHWTGGKEFKCKVHSKGLPAPIVR
TISRAKGQAREPQVYVLAPPQEELSKSTLSVTCLVTGFYPDYIAVEWQRA
RQPESEDKYGTTTSQLDADGSYFLYSRLRVDKSSWQRGDTYACVVMHEAL
HNHYTQKSISKPPGK
IgG3
(Schwartz et al, 2018, Immunogenetics, 70: 317)
(SEQ ID NO: 24)
PEPLGGLSVFIFPPKPKDTLTISGTPEVTCVVVDVGQDDPEVQFSWFVDN
VEVRTARTKPREEQFNSTFRVVSALPIQHQDWLRGKEIKCKVHNKGLPAP
IVRTISRTKGQAREPQVYVLAPPQEELSKSTLSVTCLVTGFYPDYIAVEW
QKNGQPESEDKYGTTTSQLDADGSYFLYSRLRVDKNSWQEGDTYACVVMH
EALHNHYTQKSISKPPGK

The CH2 region of an ovine antibody comprises or consists of amino acids 231 to 340 (according to EU numbering) of an ovine IgG antibody. It is to be understood that the CH2 region may include one to six (e.g., 1, 2, 3, 4, 5, 6) additional amino acids or deletions at their N and/or C-terminus.

The CH3 region of an ovine antibody comprises or consists of amino acids 341 to 447 (according to EU numbering) of an ovine IgG antibody. It is to be understood that the CH3 region may include one to six (e.g., 1, 2, 3, 4, 5, 6) additional amino acids or deletions at their N and/or C-terminus.

The Fc region of an ovine IgG antibody comprises or consists of amino acids 231 to 447 (according to EU numbering) of an ovine IgG antibody.

Table 4 below compares the amino acid sequences of the CH2 and CH3 domains of human IgG1, canine IgGB, and feline IgG1a with each of the three ovine IgG isotypes, based on EU numbering:

TABLE 4

| EU | Human | Canine | Feline | Sheep | Sheep | Sheep |
|---|---|---|---|---|---|---|
| number | IgG1 | IgGB | IgG1a | IgG1 | IgG2 | IgG3 |
| | CH2 Domain | | | | | |
| 231 | A | A | P | | | |
| 232 | P | P | P | P | | P |
| 233 | E | E | E | E | | E |
| 234 | L | M | M | L | | P |
| 235 | L | L | L | P | V | L |
| 236 | G | G | G | G | S | G |
| 237 | G | G | G | G | R | G |
| 238 | P | P | P | P | P | L |
| 239 | S | S | S | S | S | S |
| 240 | V | V | I | V | V | V |
| 241 | F | F | F | F | F | F |
| 242 | L | I | I | I | I | I |
| 243 | F | F | F | F | F | F |
| 244 | P | P | P | P | P | P |
| 245 | P | P | P | P | P | P |
| 246 | K | K | K | K | K | K |
| 247 | P | P | P | P | P | P |
| 248 | K | K | K | K | K | K |
| 249 | D | D | D | D | D | D |
| 250 | T | T | T | T | S | T |
| 251 | L | L | L | L | L | L |
| 252 | M | L | S | T | M | T |
| 253 | I | I | I | I | I | I |
| 254 | S | A | S | S | T | S |
| 255 | R | R | R | G | G | G |
| 256 | T | T | T | T | T | T |
| 257 | P | P | P | P | P | P |
| 258 | E | E | E | E | E | E |
| 259 | V | V | V | V | V | V |
| 260 | T | T | T | T | T | T |
| 261 | C | C | C | C | C | C |
| 262 | V | V | L | V | V | V |
| 263 | V | V | V | V | V | V |
| 264 | V | V | V | V | V | V |
| 265 | D | D | D | D | D | D |
| 266 | V | L | L | V | V | V |
| 267 | S | D | G | G | G | G |
| 268 | H | P | P | Q | Q | Q |
| 269 | E | E | D | D | G | D |
| 270 | D | D | D | D | D | D |
| 271 | P | P | S | P | P | P |
| 272 | E | E | D | E | E | E |
| 273 | V | V | V | V | V | V |
| 274 | K | Q | Q | Q | Q | Q |
| 275 | F | I | I | F | F | F |
| 276 | N | S | T | S | S | S |
| 277 | W | W | W | W | W | W |
| 278 | Y | F | F | F | F | F |
| 279 | V | V | V | V | V | V |
| 280 | D | D | D | D | D | D |
| 282 | G | G | N | N | N | N |
| 282 | V | K | T | V | V | V |
| 283 | E | Q | Q | E | E | E |
| 284 | V | M | V | V | V | V |
| 285 | H | Q | Y | R | R | R |
| 286 | N | T | T | T | T | T |
| 287 | A | A | A | A | A | A |
| 288 | K | K | K | R | R | R |
| 289 | T | T | T | T | T | T |
| 290 | K | Q | S | K | K | K |
| 291 | P | P | P | P | P | P |
| 292 | R | R | R | R | R | R |
| 293 | E | E | E | E | E | E |
| 294 | E | E | E | E | E | E |
| 295 | Q | Q | Q | Q | Q | Q |
| 296 | Y | F | F | F | F | F |
| 297 | N | N | N | N | N | N |
| 298 | S | G | S | S | S | S |
| 299 | T | T | T | T | T | T |
| 300 | Y | Y | Y | F | F | F |
| 301 | R | R | R | R | R | R |
| 302 | V | V | V | V | V | V |
| 303 | V | V | V | V | V | V |
| 304 | S | S | S | S | S | S |
| 305 | V | V | V | A | A | A |
| 306 | L | L | L | L | L | L |
| 307 | T | P | P | P | P | P |
| 308 | V | I | I | I | I | I |
| 309 | L | G | L | Q | Q | Q |
| 310 | H | H | H | H | H | H |
| 311 | Q | Q | Q | Q | D | Q |
| 312 | D | D | D | D | H | D |
| 313 | W | W | W | W | W | W |
| 314 | L | L | L | T | T | L |
| 315 | N | K | K | G | G | R |
| 316 | G | G | G | G | G | G |
| 317 | K | K | K | K | K | K |
| 318 | E | Q | E | E | E | E |
| 319 | Y | F | F | F | F | I |
| 320 | K | T | K | K | K | K |
| 321 | C | C | C | C | C | C |
| 322 | K | K | K | K | K | K |
| 323 | V | V | V | V | V | V |
| 324 | S | N | N | H | H | H |
| 325 | N | N | S | N | S | N |
| 326 | K | K | K | E | K | K |
| 327 | A | A | S | A | G | G |
| 328 | L | L | L | L | L | L |
| 329 | P | P | P | P | P | P |
| 330 | A | S | S | A | A | A |
| 331 | P | P | P | P | P | P |
| 332 | I | I | I | I | I | I |
| 333 | E | E | E | V | V | V |
| 334 | K | R | R | R | R | R |
| 335 | T | T | T | T | T | T |
| 336 | I | I | I | I | I | I |
| 337 | S | S | S | S | S | S |
| 338 | K | K | K | R | R | R |
| 339 | A | A | A | T | A | T |
| 340 | K | R | K | K | K | K |
| | CH3 Domain | | | | | |
| 341 | G | G | G | G | G | G |
| 342 | Q | Q | Q | Q | Q | Q |
| 343 | P | A | P | A | A | A |
| 344 | R | H | H | R | R | R |
| 345 | E | Q | E | E | E | E |
| 346 | P | P | P | P | P | P |
| 347 | Q | s | Q | Q | Q | Q |
| 348 | V | V | V | V | V | V |
| 349 | Y | Y | Y | Y | Y | Y |
| 350 | T | V | V | V | V | V |
| 351 | L | L | L | L | L | L |
| 352 | P | P | P | A | A | A |
| 353 | P | P | P | P | P | P |
| 354 | S | S | A | P | P | P |
| 355 | R | R | Q | Q | Q | Q |
| 356 | D | E | E | E | E | E |
| 357 | E | E | E | E | E | E |
| 358 | L | L | L | L | L | L |
| 359 | T | S | S | S | S | S |
| 360 | K | K | R | K | K | K |
| 361 | N | N | N | S | S | S |
| 362 | Q | T | K | T | T | T |
| 363 | V | V | V | L | L | L |
| 364 | S | S | S | S | S | S |
| 365 | L | L | V | V | V | V |
| 366 | T | T | T | T | T | T |
| 367 | C | C | C | C | C | C |
| 368 | L | L | L | L | L | L |
| 369 | V | I | I | V | V | V |
| 370 | K | K | K | T | T | T |
| 371 | G | D | S | G | G | G |
| 372 | F | F | F | F | F | F |
| 373 | Y | F | H | Y | Y | Y |
| 374 | P | P | P | P | P | P |
| 375 | S | P | P | D | D | D |
| 376 | D | D | D | Y | Y | Y |
| 377 | I | I | I | I | I | I |
| 378 | A | D | A | A | A | A |
| 379 | V | V | V | V | V | V |
| 380 | E | E | E | E | E | E |
| 381 | W | W | W | W | W | W |
| 382 | E | Q | E | Q | Q | Q |
| 383 | S | S | I | K | R | K |
| 384 | N | N | T | N | A | N |
| 385 | G | G | G | G | R | G |
| 386 | Q | Q | Q | Q | Q | Q |
| 387 | P | Q | P | P | P | P |
| 388 | E | E | E | E | E | E |
| 389(a) | N | P | P | S | S | S |
| 389b | | E | E | E | E | E |
| 389c | | S | N | D | D | D |
| 390 | N | K | N | K | K | K |
| 391 | Y | Y | Y | Y | Y | Y |
| 392 | K | R | R | G | G | G |
| 393 | T | T | T | T | T | T |
| 394 | T | T | T | T | T | T |
| 395 | P | P | P | T | T | T |
| 396 | P | P | P | S | S | S |
| 397 | v | Q | Q | Q | Q | Q |
| 398 | L | L | L | L | L | L |
| 399 | D | D | D | D | D | D |
| 400 | S | E | S | A | A | A |
| 401 | D | D | D | D | D | D |
| 402 | G | G | G | G | G | G |
| 403 | S | S | T | S | S | S |
| 404 | F | Y | Y | Y | Y | Y |
| 405 | F | F | F | F | F | F |
| 406 | L | L | V | L | L | L |
| 407 | Y | Y | Y | Y | Y | Y |
| 408 | 5 | 5 | 5 | 5 | 5 | 5 |
| 409 | K | K | K | R | R | R |
| 410 | L | L | L | L | L | L |
| 411 | T | S | S | R | R | R |
| 412 | V | V | V | V | V | V |
| 413 | D | D | D | D | D | D |
| 414 | K | K | R | K | K | K |
| 415 | S | S | S | N | S | N |
| 416 | R | R | H | S | S | S |
| 417 | W | W | W | W | W | W |
| 418 | Q | Q | Q | Q | Q | Q |
| 419 | Q | R | R | E | R | E |
| 420 | G | G | G | G | G | G |
| 421 | N | D | N | D | D | D |
| 422 | V | T | T | T | T | T |
| 423 | F | F | Y | Y | Y | Y |
| 424 | S | I | T | A | A | A |
| 425 | C | C | C | C | C | C |
| 426 | S | A | S | V | V | V |
| 427 | V | V | V | V | V | V |
| 428 | M | M | S | M | M | M |
| 429 | H | H | H | H | H | H |
| 430 | E | E | E | E | E | E |
| 431 | A | A | A | A | A | A |
| 432 | L | L | L | L | L | L |
| 433 | H | H | H | H | H | H |
| 434 | N | N | S | N | N | N |
| 435 | H | H | H | H | H | H |
| 436 | Y | Y | H | Y | Y | Y |
| 437 | T | T | T | T | T | T |
| 438 | Q | Q | Q | Q | Q | Q |
| 439 | K | E | K | K | K | K |
| 440 | S | S | S | S | S | S |
| 441 | L | L | L | I | I | I |
| 442 | S | S | T | S | S | S |
| 443 | L | H | Q | K | K | K |
| 444 | S | S | S | P | P | P |
| 445 | P | P | P | P | P | P |
| 446 | G | G | G | G | G | G |
| 447 | K | K | K | K | K | K |

Substitutions in Ovine IgG Fc that Improve Half-Life

Increased serum persistence is a beneficial property for therapeutic polypeptides. This disclosure features substitutions in wild type ovine IgG1, IgG2, and IgG3 Fc regions that enhance the half-life of a polypeptide or polypeptides comprising these Fc regions in an ovine relative to a control polypeptide or control polypeptides, wherein the control polypeptide or control polypeptides are identical to the polypeptide or polypeptides except for having the corresponding wild type ovine IgG Fc region in place of the IgG Fc region variant. The substitutions to increase half-life may be made in one or more of an ovine CH2 domain, an ovine CH3 domain, or in the context of an ovine Fc (e.g., a CH2+CH3) region.

The present disclosure provides a polypeptide comprising an ovine IgG Fc region variant, or an ovine FcRn-binding region thereof, wherein the polypeptide comprises an amino acid substitution at at least one (e.g., 1, 2, 3, 4, 5, 6, 7, 8, 9, 10, or 11) position selected from the group consisting of:
(i) a position that corresponds to amino acid position 250 of a wild type ovine IgG, wherein the amino acid substitution is selected from the group consisting of S250Q, S250E, T250Q, and T250E;
(ii) a position that corresponds to amino acid position 252 of a wild type ovine IgG, wherein the amino acid substitution is selected from the group consisting of M252Y, M252W, M252F, T252Y, T252W, and T252F;
(iii) a position that corresponds to amino acid position 254 of a wild type ovine IgG wherein the amino acid substitution is selected from the group consisting of S254R, S254K, S254H, T254R, T254K, and T254H;
(iv) a position that corresponds to amino acid position 256 of a wild type ovine IgG, wherein the amino acid substitution is selected from the group consisting of T256A, T256D, and T256E;
(v) a position that corresponds to amino acid position 286 of a wild type ovine IgG, wherein the amino acid substitution is selected from the group consisting of T286Y, T286F, T286W, T286L, T286D, and T286E;
(vi) a position that corresponds to amino acid position 309 of a wild type ovine IgG, wherein the amino acid substitution is selected from the group consisting of Q309V, Q309D, and Q309E;
(vii) a position that corresponds to amino acid position 311 of a wild type ovine IgG, wherein the amino acid substitution is selected from the group consisting of Q311V, Q311K, Q311R, Q311L, Q311H, D311V, D311K, D311R, D311L, and D311H;
(viii) a position that corresponds to amino acid position 426 of a wild type ovine IgG, wherein the amino acid substitution is selected from the group consisting of V426Y, V426F, and V426H;
(ix) a position that corresponds to amino acid position 428 of a wild type ovine IgG, wherein the amino acid substitution is selected from the group consisting of M428L, M428Y, and M428F;
(x) a position that corresponds to amino acid position 434 of a wild type ovine IgG, wherein the amino acid substitution is selected from the group consisting of N434A, N434H, N434F, N434S, N434W, and N434Y; and
(xi) a position that corresponds to amino acid position 436 of a wild type ovine IgG, wherein the amino acid substitution is Y436H,
wherein the amino acid positions are based on EU numbering, and wherein the polypeptide has increased binding affinity to ovine FcRn when compared to an Fc domain of the wild type ovine IgG.

In some embodiments, the polypeptide comprises an amino acid substitution at at least one (e.g., 1, 2, 3, 4, or 5) position selected from the group consisting of:
(i) a position that corresponds to amino acid position 252 of a wild type IgG of the livestock animal, wherein the amino acid substitution is T252Y;
(ii) a position that corresponds to amino acid position 309 of a wild type IgG of the livestock animal, wherein the amino acid substitution is Q309V;
(iii) a position that corresponds to amino acid position 311 of a wild type IgG of the livestock animal, wherein the amino acid substitution is Q311V;
(iv) a position that corresponds to amino acid position 428 of a wild type IgG of the livestock animal, wherein the amino acid substitution is selected from the group consisting of M428Y; and
(v) a position that corresponds to amino acid position 434 of a wild type IgG of the livestock animal, wherein the amino acid substitution is selected from the group consisting of N434Y.

In some embodiments, the polypeptide has increased binding affinity to ovine FcRn at a pH of about 5.0 to about 6.5 (e.g., about 5.5 or about 6.0) when compared to an Fc domain of the wild type ovine IgG at the same pH.

In some embodiments, the polypeptide comprises an amino acid sequence that is at least 80% (e.g., at least 85%, 90%, 92%, 94%, 95%, 96%, 97%, 98%, or 99%) identical to an amino acid sequence selected from the group consisting of SEQ ID NOs: 22 to 24.

In some instances, this disclosure provides an ovine IgG CH2 domain variant comprising an amino acid sequence that is at least 75%, at least 80%, at least 85%, at least 90%, at least 95%, at least 96%, at least 97%, at least 98% or at least 99% identical to the CH2 domains shown in any one of SEQ ID NOs: 22 to 24. Also provided are ovine IgG CH2 domain variants comprising an amino acid sequence that varies from any one of the CH2 domains shown in SEQ ID NOs: 22 to 24 by 1 to 15 (e.g., by 1, 2, 3, 4, 5, 6, 7, 8, 9, 10, 11, 12, 13, 14, or 15) amino acids.

In some instances, this disclosure features an ovine IgG CH3 domain variant comprising an amino acid sequence that is at least 75%, at least 80%, at least 85%, at least 90%, at least 95%, at least 96%, at least 97% or at least 98% or at least 99%, identical to the amino acid sequence of the CH3 domain shown in any one of SEQ ID NOs: 22 to 24. Also featured are ovine IgG CH3 domain variants comprising an amino acid sequence that varies from any one of the CH3 domain sequences shown in any one of SEQ ID NOs: 22 to 24 by 1 to 15 (e.g., by 1, 2, 3, 4, 5, 6, 7, 8, 9, 10, 11, 12, 13, 14, or 15) amino acids.

In some instances, this disclosure features an ovine IgG Fc region variant comprising an amino acid sequence that is at least 75%, at least 80%, at least 85%, at least 90%, at least 95%, at least 96%, at least 97%, at least 98% or at least 99% identical to the amino acid sequence set forth in any one of SEQ ID NOs: 22 to 24. Also disclosed are ovine IgG Fc region variants comprising an amino acid sequence that varies from any one of SEQ ID NOs: 22 to 24 by 1 to 20 (e.g., by 1, 2, 3, 4, 5, 6, 7, 8, 9, 10, 11, 12, 13, 14, 15, 16, 17, 18, 19, or 20) amino acids.

In some embodiments, provided are a polypeptide or polypeptides comprising an ovine IgG Fc CH2 domain variant, the CH2 domain variant comprising an amino acid sequence that is at least 75%, at least 80%, at least 85%, at least 90%, at least 95%, at least 96%, at least 97%, at least 98% or at least 99%, identical to the amino acid sequence of the CH2 domain shown in any one of SEQ ID NOs: 22 to 24.

In some embodiments, featured are a polypeptide or polypeptides comprising an ovine IgG Fc CH3 domain variant, the CH3 domain variant comprising an amino acid sequence that is at least 75%, at least 80%, at least 85%, at least 90%, at least 95%, at least 96%, at least 97%, at least 98% or at least 99% identical to the amino acid sequence of the CH3 domain shown in any one of SEQ ID NOs: 22 to 24.

In some embodiments, featured are a polypeptide or polypeptides comprising an ovine IgG Fc region variant, the Fc region variant comprising an amino acid sequence that is at least 75%, at least 80%, at least 85%, at least 90%, at least 95%, at least 96%, at least 97%, at least 98% or at least 99% identical to the amino acid sequence set forth in any one of SEQ ID NOs: 22 to 24.

### Caprine Antibodies

Caprine typically have three IgG heavy chains referred to as IgG1, IgG2, and IgG3. These heavy chains represent three different subclasses of caprine IgG. The amino acid and DNA sequences for these heavy chains are available from the GENBANK® database. Illustrative examples of the amino acid sequences of the CH2 and CH3 domains of each of the three caprine IgG heavy chain Fc regions are shown below (CH2 domains are underlined):

Goat (Capra hircus)
IgG1 (Schwartz et al, 2018, Immunogenetics, 70:317)
(SEQ ID NO: 25)
PELPGGPSVFIFPPKPKDTLTISGKPEVTCVVVDVGQDDPEVQFSWFVDNV
EVHTARTKPREEQFNSTFRVVSALPIQHQDWTGGKEFKCKVHNEGLPAPIV
RTISRTKGQAREPQVYVLAPPQEELSKSTLSVTCLVTGFYPDYIAVEWQRN
GQPESEDKYGTTTSQLDADGSYFLYSRLRVNKSSWQEGDTYACVVMHEALH
NHYTQKSISKPPGK
IgG2 (Schwartz et al, 2018, Immunogenetics, 70:317)
(SEQ ID NO: 26)
VRGPSVFIFPPKPKDSLMITGTPEVTCVVVDVGQDDPEVQFSWFVDNVEVH
TARTKPREEQFNSTFRVVSALPIQHDHWTGGKEFKCKVNNKALPAPIVRTI
SRDKGQAREPQVYVLAPPQEELSKSTLSVTCLVTGFYPDYIAVEWQRARQP
ESEDKYGTTTSQLDADGSYFLYSRLRVDKSSWQEGDTYACVVMHEALHNHY
TQKSISKPPGK
IgG3 (Schwartz et al, 2018, Immunogenetics, 70:317)
(SEQ ID NO: 27)
PEPLGGLSVFIFPPKPKDTLTISGTPEVTCVVVDVGQDDPEVQFSWFMDNV
EVHTARTTPREEQFNSTFRVVSALPIQHKDWLQGKEFKCKVHNEGLPAPII
RTISRAKGQAREPQVYVLAPPREELSKSTLSVTCLITGFYPEEVDVEWQRD
GQPESEDKYHTAPPQLDADGSYFLYSRLRVNKSSWQEGDTYTCAVMHEALR
NHYKEKSISKSPGK

The CH2 region of a caprine antibody comprises or consists of amino acids 231 to 340 (according to EU numbering) of a caprine IgG antibody. It is to be understood that the CH2 region may include one to six (e.g., 1, 2, 3, 4, 5, 6) additional amino acids or deletions at their N and/or C-terminus.

The CH3 region of a caprine antibody comprises or consists of amino acids 341 to 447 (according to EU numbering) of a caprine IgG antibody. It is to be understood that the CH3 region may include one to six (e.g., 1, 2, 3, 4, 5, 6) additional amino acids or deletions at their N and/or C-terminus.

The Fc region of a caprine IgG antibody comprises or consists of amino acids 231 to 447 (according to EU numbering) of a caprine IgG antibody.

Table 5 below compares the amino acid sequences of the CH2 and CH3 domains of human IgG1, canine IgGB, and feline IgG1a with each of the three caprine IgG isotypes, based on EU numbering:

TABLE 5

| EU | Human | Canine | Feline | Goat | Goat | Goat |
|---|---|---|---|---|---|---|
| number | IgG1 | IgGB | IgGla | IgG1 | IgG2 | IgG3 |
| | CH2 Domain | | | | | |
| 231 | A | A | P | | | |
| 232 | P | P | P | P | | P |
| 233 | E | E | E | E | | E |
| 234 | L | M | M | L | | P |
| 235 | L | L | L | P | V | L |
| 236 | G | G | G | G | R | G |
| 237 | G | G | G | G | G | G |
| 238 | P | P | P | P | P | L |
| 239 | S | S | S | S | S | S |
| 240 | V | V | I | V | V | V |
| 241 | F | F | F | F | F | F |
| 242 | L | I | I | I | I | I |
| 243 | F | F | F | F | F | F |
| 244 | P | P | P | P | P | P |
| 245 | P | P | P | P | P | P |
| 246 | K | K | K | K | K | K |
| 247 | P | P | P | P | P | P |
| 248 | K | K | K | K | K | K |
| 249 | D | D | D | D | D | D |
| 250 | T | T | T | T | S | T |
| 251 | L | L | L | L | L | L |
| 252 | M | L | S | T | M | T |
| 253 | I | I | I | I | I | I |
| 254 | S | A | S | S | T | S |
| 255 | R | R | R | G | G | G |
| 256 | T | T | T | K | T | T |
| 257 | P | P | P | P | P | P |
| 258 | E | E | E | E | E | E |
| 259 | V | V | V | V | V | V |
| 260 | T | T | T | T | T | T |
| 261 | C | C | C | C | C | C |
| 262 | V | V | L | V | V | V |
| 263 | V | V | V | V | V | V |
| 264 | V | V | V | V | V | V |
| 265 | D | D | D | D | D | D |
| 266 | V | L | L | V | V | V |
| 267 | S | D | G | G | G | G |
| 268 | H | P | P | Q | Q | Q |
| 269 | E | E | D | D | D | D |
| 270 | D | D | D | D | D | D |
| 271 | P | P | S | P | P | P |
| 272 | E | E | D | E | E | E |
| 273 | V | V | V | V | V | V |
| 274 | K | Q | Q | Q | Q | Q |
| 275 | F | I | I | F | F | F |
| 276 | N | S | T | S | S | S |
| 277 | W | W | W | W | W | W |
| 278 | Y | F | F | F | F | F |
| 279 | V | V | V | V | V | M |
| 280 | D | D | D | D | D | D |
| 282 | G | G | N | N | N | N |
| 282 | V | K | T | V | V | V |
| 283 | E | Q | Q | E | E | E |
| 284 | V | M | V | V | V | V |
| 285 | H | Q | Y | H | H | H |
| 286 | N | T | T | T | T | T |
| 287 | A | A | A | A | A | A |
| 288 | K | K | K | R | R | R |
| 289 | T | T | T | T | T | T |
| 290 | K | Q | S | K | K | T |
| 291 | P | P | P | P | P | P |
| 292 | R | R | R | R | R | R |
| 293 | E | E | E | E | E | E |
| 294 | E | E | E | E | E | E |
| 295 | Q | Q | Q | Q | Q | Q |
| 296 | Y | F | F | F | F | F |
| 297 | N | N | N | N | N | N |
| 298 | S | G | S | S | S | S |
| 299 | T | T | T | T | T | T |
| 300 | Y | Y | Y | F | F | F |
| 301 | R | R | R | R | R | R |
| 302 | V | V | V | V | V | V |
| 303 | V | V | V | V | V | V |
| 304 | S | S | S | S | S | S |
| 305 | V | V | V | A | A | A |
| 306 | L | L | L | L | L | L |
| 307 | T | P | P | P | P | P |
| 308 | V | I | I | I | I | I |
| 309 | L | G | L | Q | Q | Q |
| 310 | H | H | H | H | H | H |
| 311 | Q | Q | Q | Q | D | K |
| 312 | D | D | D | D | H | D |
| 313 | W | W | W | W | W | W |
| 314 | L | L | L | T | T | L |
| 315 | N | K | K | G | G | Q |
| 316 | G | G | G | G | G | G |
| 317 | K | K | K | K | K | K |
| 318 | E | Q | E | E | E | E |
| 319 | Y | F | F | F | F | F |
| 320 | K | T | K | K | K | K |
| 321 | C | C | C | C | C | C |
| 322 | K | K | K | K | K | K |
| 323 | V | V | V | V | V | V |
| 324 | S | N | N | H | N | H |
| 325 | N | N | S | N | N | N |
| 326 | K | K | K | E | K | E |
| 327 | A | A | S | G | A | G |
| 328 | L | L | L | L | L | L |
| 329 | P | P | P | P | P | P |
| 330 | A | S | S | A | A | A |
| 331 | P | P | P | P | P | P |
| 332 | I | I | I | I | I | I |
| 333 | E | E | E | V | V | I |
| 334 | K | R | R | R | R | R |
| 335 | T | T | T | T | T | T |
| 336 | I | I | I | I | I | I |
| 337 | S | S | S | S | S | S |
| 338 | K | K | K | R | R | R |
| 339 | A | A | A | T | D | A |
| 340 | K | R | K | K | K | K |
| | CH3 Domain | | | | | |
| 341 | G | G | G | G | G | G |
| 342 | Q | Q | Q | Q | Q | Q |
| 343 | P | A | P | A | A | A |
| 344 | R | H | H | R | R | R |
| 345 | E | Q | E | E | E | E |
| 346 | P | P | P | P | P | P |
| 347 | Q | s | Q | Q | Q | Q |
| 348 | V | V | V | V | V | V |
| 349 | Y | Y | Y | Y | Y | Y |
| 350 | T | V | V | V | V | V |
| 351 | L | L | L | L | L | L |
| 352 | P | P | P | A | A | A |
| 353 | P | P | P | P | P | P |
| 354 | S | S | A | P | P | P |
| 355 | R | R | Q | Q | Q | R |
| 356 | D | E | E | E | E | E |
| 357 | E | E | E | E | E | E |
| 358 | L | L | L | L | L | L |
| 359 | T | S | S | S | S | S |
| 360 | K | K | R | K | K | K |
| 361 | N | N | N | S | S | S |
| 362 | Q | T | K | T | T | T |
| 363 | V | V | V | L | L | L |
| 364 | S | S | S | S | S | S |
| 365 | L | L | V | V | V | V |
| 366 | T | T | T | T | T | T |
| 367 | C | C | C | C | C | C |
| 368 | L | L | L | L | L | L |
| 369 | V | I | I | V | V | I |
| 370 | K | K | K | T | T | T |
| 371 | G | D | S | G | G | G |
| 372 | F | F | F | F | F | F |
| 373 | Y | F | H | Y | Y | Y |
| 374 | P | P | P | P | P | P |
| 375 | S | P | P | D | D | E |
| 376 | D | D | D | Y | Y | E |
| 377 | I | I | I | I | I | V |
| 378 | A | D | A | A | A | D |
| 379 | V | V | V | V | V | V |
| 380 | E | E | E | E | E | E |
| 381 | W | W | W | W | W | W |
| 382 | E | Q | E | Q | Q | Q |
| 383 | S | S | I | R | R | R |
| 384 | N | N | T | N | A | D |
| 385 | G | G | G | G | R | G |
| 386 | Q | Q | Q | Q | Q | Q |
| 387 | P | Q | P | P | P | P |
| 388 | E | E | E | E | E | E |
| 389(a) | N | P | P | S | S | S |
| 389b | | E | E | E | E | E |
| 389c | | S | N | D | D | D |
| 390 | N | K | N | K | K | K |
| 391 | Y | Y | Y | Y | Y | Y |
| 392 | K | R | R | G | G | H |
| 393 | T | T | T | T | T | T |
| 394 | T | T | T | T | T | A |
| 395 | P | P | P | T | T | P |
| 396 | P | P | P | S | S | P |
| 397 | v | Q | Q | Q | Q | Q |
| 398 | L | L | L | L | L | L |
| 399 | D | D | D | D | D | D |
| 400 | S | E | S | A | A | A |
| 401 | D | D | D | D | D | D |
| 402 | G | G | G | G | G | G |
| 403 | S | S | T | S | S | S |
| 404 | F | Y | Y | Y | Y | Y |
| 405 | F | F | F | F | F | F |
| 406 | L | L | V | L | L | L |
| 407 | Y | Y | Y | Y | Y | Y |
| 408 | S | S | S | S | S | S |
| 409 | K | K | K | R | R | R |
| 410 | L | L | L | L | L | L |
| 411 | T | S | S | R | R | R |
| 412 | V | V | V | V | V | V |
| 413 | D | D | D | N | D | N |
| 414 | K | K | R | K | K | K |
| 415 | S | S | S | S | S | S |
| 416 | R | R | H | S | S | S |
| 417 | W | W | W | W | W | W |
| 418 | Q | Q | Q | Q | Q | Q |
| 419 | Q | R | R | E | E | E |
| 420 | G | G | G | G | G | G |
| 421 | N | D | N | D | D | D |
| 422 | V | T | T | T | T | T |
| 423 | F | F | Y | Y | Y | Y |
| 424 | S | I | T | A | A | T |
| 425 | C | C | C | C | C | C |
| 426 | S | A | S | V | V | A |
| 427 | V | V | V | V | V | V |
| 428 | M | M | S | M | M | M |
| 429 | H | H | H | H | H | H |
| 430 | E | E | E | E | E | E |
| 431 | A | A | A | A | A | A |
| 432 | L | L | L | L | L | L |
| 433 | H | H | H | H | H | R |
| 434 | N | N | S | N | N | N |
| 435 | H | H | H | H | H | H |
| 436 | Y | Y | H | Y | Y | Y |
| 437 | T | T | T | T | T | K |
| 438 | Q | Q | Q | Q | Q | E |
| 439 | K | E | K | K | K | K |
| 440 | S | S | S | S | S | S |
| 441 | L | L | L | I | I | I |
| 442 | S | S | T | S | S | S |
| 443 | L | H | Q | K | K | K |
| 444 | S | S | S | P | P | S |
| 445 | P | P | P | P | P | P |
| 446 | G | G | G | G | G | G |
| 447 | K | K | K | K | K | K |

Substitutions in Caprine IgG Fc that Improve Half-Life

Increased serum persistence is a beneficial property for therapeutic polypeptides. This disclosure features substitutions in wild type caprine IgG1, IgG2, and IgG3 Fc regions that enhance the half-life of a polypeptide or polypeptides comprising these Fc regions in a caprine relative to a control polypeptide or control polypeptides, wherein the control polypeptide or control polypeptides are identical to the polypeptide or polypeptides except for having the corresponding wild type caprine IgG Fc region in place of the IgG Fc region variant. The substitutions to increase half-life may be made in one or more of a caprine CH2 domain, a caprine CH3 domain, or in the context of a caprine Fc (e.g., a CH2+CH3) region.

The present disclosure provides a polypeptide comprising a caprine IgG Fc region variant, or a caprine FcRn-binding region thereof, wherein the polypeptide comprises an amino acid substitution at at least one (e.g., 1, 2, 3, 4, 5, 6, 7, 8, 9, 10, or 11) position selected from the group consisting of:
(i) a position that corresponds to amino acid position 250 of a wild type caprine IgG, wherein the amino acid substitution is selected from the group consisting of S250Q, S250E, T250Q, and T250E;
(ii) a position that corresponds to amino acid position 252 of a wild type caprine IgG, wherein the amino acid substitution is selected from the group consisting of M252Y, M252W, M252F, T252Y, T252W, and T252F;
(iii) a position that corresponds to amino acid position 254 of a wild type caprine IgG, wherein the amino acid substitution is selected from the group consisting of S254R, S254K, S254H, T254R, T254K, and T254H;
(iv) a position that corresponds to amino acid position 256 of a wild type caprine IgG, wherein the amino acid substitution is selected from the group consisting of T256A, T256D, T256E, K256A, K256D, and K256E;
(v) a position that corresponds to amino acid position 286 of a wild type caprine IgG, wherein the amino acid substitution is selected from the group consisting of T286Y, T286F, T286W, T286L, T286D, and T286E;
(vi) a position that corresponds to amino acid position 309 of a wild type caprine IgG, wherein the amino acid substitution is selected from the group consisting of Q309V, Q309D, and Q309E;
(vii) a position that corresponds to amino acid position 311 of a wild type caprine IgG, wherein the amino acid substitution is selected from the group consisting of Q311V, Q311K, Q311R, Q311L, Q311H, K311V, K311R, K311L, K311H, D311V, D311K, D311R, D311L, and D311H;
(viii) a position that corresponds to amino acid position 426 of a wild type caprine IgG wherein the amino acid substitution is selected from the group consisting of A426Y, A426F, A426H, V426Y, V426F, and V426H;
(ix) a position that corresponds to amino acid position 428 of a wild type caprine IgG, wherein the amino acid substitution is selected from the group consisting of M428L, M428Y, and M428F;
(x) a position that corresponds to amino acid position 434 of a wild type caprine IgG, wherein the amino acid substitution is selected from the group consisting of N434A, N434H, N434F, N434S, N434W, and N434Y; and
(xi) a position that corresponds to amino acid position 436 of a wild type caprine IgG, wherein the amino acid substitution is Y436H,
wherein the amino acid positions are based on EU numbering, and wherein the polypeptide has increased binding affinity to caprine FcRn when compared to an Fc domain of the wild type caprine IgG.

In some embodiments, the polypeptide has increased binding affinity to caprine FcRn at a pH of about 5.0 to about 6.5 (e.g., about 5.5 or about 6.0) when compared to an Fc domain of the wild type caprine IgG at the same pH.

In some embodiments, the polypeptide comprises an amino acid sequence that is at least 80% (e.g., at least 85%, 90%, 92%, 94%, 95%, 96%, 97%, 98%, or 99%) identical to an amino acid sequence selected from the group consisting of SEQ ID NOs: 25 to 27.

In some instances, this disclosure provides a caprine IgG CH2 domain variant comprising an amino acid sequence that is at least 75%, at least 80%, at least 85%, at least 90%, at least 95%, at least 96%, at least 97%, at least 98% or at least 99% identical to the CH2 domains shown in any one of SEQ ID NOs: 25 to 27. Also provided are caprine IgG CH2 domain variants comprising an amino acid sequence that varies from any one of the CH2 domains shown in SEQ ID NOs: 25 to 27 by 1 to 15 (e.g., by 1, 2, 3, 4, 5, 6, 7, 8, 9, 10, 11, 12, 13, 14, or 15) amino acids.

In some instances, this disclosure features a caprine IgG CH3 domain variant comprising an amino acid sequence that is at least 75%, at least 80%, at least 85%, at least 90%, at least 95%, at least 96%, at least 97% or at least 98% or at least 99%, identical to the amino acid sequence of the CH3 domain shown in any one of SEQ ID NOs: 25 to 27. Also featured are caprine IgG CH3 domain variants comprising an amino acid sequence that varies from any one of the CH3 domain sequences shown in any one of SEQ ID NOs: 25 to 27 by 1 to 15 (e.g., by 1, 2, 3, 4, 5, 6, 7, 8, 9, 10, 11, 12, 13, 14, or 15) amino acids.

In some instances, this disclosure features a caprine IgG Fc region variant comprising an amino acid sequence that is at least 75%, at least 80%, at least 85%, at least 90%, at least 95%, at least 96%, at least 97%, at least 98% or at least 99% identical to the amino acid sequence set forth in any one of SEQ ID NOs: 25 to 27. Also disclosed are caprine IgG Fc region variants comprising an amino acid sequence that varies from any one of SEQ ID NOs: 25 to 27 by 1 to 20 (e.g., by 1, 2, 3, 4, 5, 6, 7, 8, 9, 10, 11, 12, 13, 14, 15, 16, 17, 18, 19, or 20) amino acids.

In some embodiments, provided are a polypeptide or polypeptides comprising a caprine IgG Fc CH2 domain variant, the CH2 domain variant comprising an amino acid sequence that is at least 75%, at least 80%, at least 85%, at least 90%, at least 95%, at least 96%, at least 97%, at least 98% or at least 99%, identical to the amino acid sequence of the CH2 domain shown in any one of SEQ ID NOs: 25 to 27.

In some embodiments, featured are a polypeptide or polypeptides comprising a caprine IgG Fc CH3 domain variant, the CH3 domain variant comprising an amino acid sequence that is at least 75%, at least 80%, at least 85%, at least 90%, at least 95%, at least 96%, at least 97%, at least 98% or at least 99% identical to the amino acid sequence of the CH3 domain shown in any one of SEQ ID NOs: 25 to 27.

In some embodiments, featured are a polypeptide or polypeptides comprising a caprine IgG Fc region variant, the Fc region variant comprising an amino acid sequence that is at least 75%, at least 80%, at least 85%, at least 90%, at least 95%, at least 96%, at least 97%, at least 98% or at least 99% identical to the amino acid sequence set forth in any one of SEQ ID NOs: 25 to 27.

The present disclosure extends to a polypeptide or polypeptides in which the amino acid substitutions are made on either one or both of a CH2 domain and a CH3 domain. In some instances, the substitutions on the CH2 domain and/or the CH3 domain are identical. In some instances, the substitutions on the CH2 domain and/or the CH3 domain are not identical. In some instances, the Fc region includes one or more additional substitutions that increase or decrease effector function and/or improve product heterogeneity.

Other Substitutions that can be Combined with the Half-Life Enhancing Substitutions

The development of a therapeutic polypeptide/protein (e.g., a monoclonal antibody) is a complex process that entails coordination of a complex set of activities to generate the desired polypeptide/protein. These include optimization of the specificity, affinity, functional activity, expression level in engineered cell lines, long-term stability, elimination or enhancement of effector functions and development of commercially viable manufacturing and purification methods. This disclosure encompasses any additional substitution that facilitates any one or more of the above goals.

In some embodiments, substitutions are introduced to a wild type IgG Fc region of the livestock animal to enhance binding to Protein A so as to facilitate purification by protein A chromatography. Such substitutions will be familiar to persons skilled in the art and may be at one or more (e.g., 1, 2, 3, 4, 5, 6, or 7) positions of the IgG.

In some embodiments, additional amino acid substitutions can be made to alter binding affinity to FcRn as compared to a parent polypeptide or a wild-type polypeptide (e.g., to increase or reduce binding affinity with FcRn).

In some embodiments, the polypeptide comprises a hinge region of an antibody of the livestock animal. In some embodiments, modifications can be made to the hinge region of the antibody of the livestock animal to increase half-life. Such modifications will be known to persons skilled in the art.

### Polypeptides Comprising the IgG Fc Variants

The disclosure encompasses any polypeptide that may benefit from having an increased half-life in a livestock animal. To increase half-life these polypeptides are designed to include an Fc region variant (e.g., a CH2 region, a CH3 region, a CH2+CH3 region) disclosed above.

Exemplary polypeptides include, but are not limited to, whole antibodies, scFvs, nanobodies, ligand-binding portions of a receptor, cytokines, growth factors, enzymes, and peptides. For example, a CH3 domain variant disclosed above may be attached to an scFv nanobody, ligand-binding portion of a receptor (e.g., the ligand-binding portion of IL-13Rα1 or IL-13Rα2), a cytokine, a growth factor, an enzyme, or a peptide. As used herein, the terms “nanobody”, “VHH”, “VHH antibody fragment”, and “single domain antibody” are used interchangeably herein to denote the variable domain of the single heavy chain of antibodies of the type of those found in Camelidae, which are typically found in nature to lack light chains. Suitable nanobodies will be familiar to persons skilled in the art, illustrated examples of which include nanobodies of camels, dromedaries, llamas, and alpacas. Alternatively, an Fc region variant disclosed above may be attached to these polypeptides. In another embodiment, an antibody from the livestock animal is modified to include an Fc region variant disclosed herein.

In some embodiments, the polypeptides of this disclosure include an antibody hinge region. The hinge region may be placed between the antigen or ligand-binding domain of the polypeptide and the Fc region variant. In some instances, the hinge region is attached to the C-terminus of a cytokine, a growth factor, an enzyme, or a peptide and the hinge region is attached to the N-terminus of the Fc region variant. Suitable hinge region sequences will be familiar to persons skilled in the art.

The hinge region, if used, in a polypeptide of this disclosure may include zero to six (i.e., 0, 1, 2, 3, 4, 5, or 6) amino acid substitutions relative to an amino acid sequence of the native hinge region. In some instances, the hinge region used in a recombinant protein of this disclosure is at least 80%, at least 85%, at least 90%, at least 95%, at least 96%, at least 97%, at least 98%, at least 99%, or 100% identical to an amino acid sequence of the native hinge region.

The polypeptide or polypeptides of this disclosure may comprise a binding domain. The binding domain can specifically bind to a protein, subunit, domain, motif, and/or epitope of a selected target described herein. In some embodiments, the polypeptide or polypeptides (e.g., fusion polypeptide) can comprise a protein, wherein the protein is a therapeutic protein described herein. In some embodiments, the target (e.g., for the target of the binding domain) or the therapeutic protein (e.g., for the fusion polypeptide) is selected from the group consisting of: 17-IA, 4-1BB, 4Dc, 6-keto-PGF1a, 8-iso-PGF2a, 8-oxo-dG, A1 Adenosine Receptor, A33, ACE, ACE-2, Activin, Activin A, Activin AB, Activin B, Activin C, Activin RIA, Activin RIA ALK-2, Activin RIB ALK-4, Activin RIIA, Activin RIIB, ADAM, ADAM10, ADAM12, ADAM15, ADAM17/TACE, ADAMS, ADAM9, ADAMTS, ADAMTS4, ADAMTS5, Addressins, aFGF, ALCAM, ALK, ALK-1, ALK-7, alpha-1-antitrypsin, alpha-V/beta-1 antagonist, ANG, Ang, APAF-1, APE, APJ, APP, APRIL, AR, IgE, Angiotensin type 1 (AT1) receptor, Angiotensin type 2 (AT2) receptor, ARC, ART, Artemin, anti-Id, ASPARTIC, Atrial natriuretic factor, av/b3 integrin, Axl, b2M, B7-1, B7-2, B7-H, B-lymphocyte Stimulator (BlyS), BACE, BACE-1, Bad, BAFF, BAFF-R, Bag-1, BAK, Bax, BCA-1, BCAM, Bcl, BCMA, BDNF, b-ECGF, bFGF, BID, Bik, BIM, BLC, BL-CAM, BLK, BMP, BMP-2 BMP-2a, BMP-3 Osteogenin, BMP-4 BMP-2b, BMP-5, BMP-6 Vgr-1, BMP-7 (OP-1), BMP-8 (BMP-8a, OP-2), BMPR, BMPR-IA (ALK-3), BMPR-IB (ALK-6), BRK-2, RPK-1, BMPR-II (BRK-3), BMPs, b-NGF, BOK, Bombesin, Bone-derived neurotrophic factor, BPDE, BPDE-DNA, BTC, complement factor 3 (C3), C3a, C4, C5, C5a, C10, CA125, CAD-8, Calcitonin, cAMP, carcinoembryonic antigen (CEA), carcinoma-associated antigen, Cathepsin A, Cathepsin B, Cathepsin C/DPPI, Cathepsin D, Cathepsin E, Cathepsin H, Cathepsin L, Cathepsin O, Cathepsin S, Cathepsin V, Cathepsin X/Z/P, CBL, CC1, CCK2, CCL, CCL1, CCL11, CCL12, CCL13, CCL14, CCL15, CCL16, CCL17, CCL18, CCL19, CCL2, CCL20, CCL21, CCL22, CCL23, CCL24, CCL25, CCL26, CCL27, CCL28, CCL3, CCL4, CCL5, CCL6, CCL7, CCL8, CCL9/10, CCR, CCR1, CCR10, CCR10, CCR2, CCR3, CCR4, CCR5, CCR6, CCR7, CCR8, CCR9, CD1, CD2, CD3, CD3E, CD4, CD5, CD6, CD7, CD8, CD10, CD11a, CD11b, CD11c, CD13, CD14, CD15, CD16, CD18, CD19, CD20, CD21, CD22, CD23, CD25, CD27L, CD28, CD29, CD30, CD30L, CD32, CD33 (p67 proteins), CD34, CD38, CD40, CD40L, CD44, CD45, CD46, CD47, CD49a, CD52, CD54, CD55, CD56, CD61, CD64, CD66e, CD74, CD80 (B7-1), CD89, CD95, CD123, CD137, CD138, CD140a, CD146, CD147, CD148, CD152, CD164, CEACAM5, CFTR, cGMP, CINC, Clostridium botulinum toxin, Clostridium perfringens toxin, CKb8-1, CLC, CMV, CMV UL, CNTF, CNTN-1, COX, C-Ret, CRG-2, CT-1, CTACK, CTGF, CTLA-4, CX3CL1, CX3CR1, CXCL, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCR, CXCR1, CXCR2, CXCR3, CXCR4, CXCR5, CXCR6, cytokeratin tumor-associated antigen, DAN, DCC, DcR3, DC-SIGN, Decay accelerating factor, des(1-3)-IGF-I (brain IGF-1), Dhh, digoxin, DNAM-1, Dnase, Dpp, DPPIV/CD26, Dtk, ECAD, EDA, EDA-A1, EDA-A2, EDAR, EGF, EGFR (ErbB-1), EMA, EMMPRIN, ENA, endothelin receptor, Enkephalinase, eNOS, Eot, eotaxin1, EpCAM, Ephrin B2/EphB4, EPO, ERCC, E-selectin, ET-1, Factor IIa, Factor VII, Factor VIIIc, Factor IX, fibroblast activation protein (FAP), Fas, FcR1, FEN-1, Ferritin, FGF, FGF-19, FGF-2, FGF3, FGF-8, FGFR, FGFR-3, Fibrin, FL, FLIP, Flt-3, Flt-4, Follicle stimulating hormone, Fractalkine, FZD1, FZD2, FZD3, FZD4, FZD5, FZD6, FZD7, FZD8, FZD9, FZD10, G250, Gas 6, GCP-2, GCSF, GD2, GD3, GDF, GDF-1, GDF-3 (Vgr-2), GDF-5 (BMP-14, CDMP-1), GDF-6 (BMP-13, CDMP-2), GDF-7 (BMP-12, CDMP-3), GDF-8 (Myostatin), GDF-9, GDF-15 (MIC-1), GDNF, GDNF, GFAP, GFRa-1, GFR-alpha1, GFR-alpha2, GFR-alpha3, GITR, GLP1, GLP2, Glucagon, Glut 4, glycoprotein IIb/IIIa (GP IIb/IIIa), GM-CSF, gp130, gp72, GRO, GnRH, Growth hormone releasing factor, Hapten (NP-cap or NIP-cap), HB-EGF, HCC, HCMV gB envelope glycoprotein, HCMV) gH envelope glycoprotein, HCMV UL, Hemopoietic growth factor (HGF), Hep B gp120, heparanase, Her2, Her2/neu (ErbB-2), Her3 (ErbB-3), Her4 (ErbB-4), herpes simplex virus (HSV) gB glycoprotein, HSV gD glycoprotein, HGFA, High molecular weight melanoma-associated antigen (HMW-MAA), HIV gp120, HIV IIIB gp120 V3 loop, HLA, HLA-DR, HM1.24, HMFG PEM, HRG, Hrk, cardiac myosin, cytomegalovirus (CMV), growth hormone (GH), HVEM, 1-309, IAP, ICAM, ICAM-1, ICAM-3, ICE, ICOS, IFNg, Ig, IgA receptor, IgE, IGF, IGF binding proteins, IGF-1R, IGFBP, IGF-I, IGF-II, IL, IL-1, IL-1R, IL-2, IL-2R, IL-4, IL-4R, IL-5, IL-5R, IL-6, IL-6R, IL-8, IL-9, IL-10, IL-12, IL-13, IL-15, IL-17, IL-18, IL-18R, IL-21, IL-22, IL-23, IL-25, IL-31, IL-33, interleukin receptor (e.g., IL-1R, IL-2R, IL-4R, IL-5R, IL-6R, IL-8R, IL-9R, IL-10R, IL-12R, IL-13R, IL-15R, IL-17R, IL-18R, IL-21R, IL-22R, IL-23R, IL-25R, IL-31R, IL-33R), interferon (INF)-alpha, INF-beta, INF-gamma, Inhibin, iNOS, Insulin A-chain, Insulin B-chain, Insulin-like growth factor 1, integrin alpha2, integrin alpha3, integrin alpha4, integrin alpha4/beta1, integrin alpha4/beta7, integrin alpha5 (alphaV), integrin alpha5/beta1, integrin alpha5/beta3, integrin alpha6, integrin beta1, integrin beta2, interferon gamma, IP-10, I-TAC, JE, Kallikrein 2, Kallikrein 5, Kallikrein 6, Kallikrein 11, Kallikrein 12, Kallikrein 14, Kallikrein 15, Kallikrein L1, Kallikrein L2, Kallikrein L3, Kallikrein L4, KC, KDR, Keratinocyte Growth Factor (KGF), laminin 5, LAMP, LAP, LAP (TGF-1), Latent TGF-1, Latent TGF-1 bp1, LBP, LDGF, LECT2, Lefty, Lewis-Y antigen, Lewis-Y related antigen, LFA-1, LFA-3, Lfo, LIF, LIGHT, lipoproteins, LIX, LKN, Lptn, L-Selectin, LT-a, LT-b, LTB4, LTBP-1, Lung surfactant, Luteinizing hormone, Lymphotoxin Beta Receptor, Mac-1, MAdCAM, MAG, MAP2, MARC, MCAM, MCAM, MCK-2, MCP, M-CSF, MDC, Mer, METALLOPROTEASES, MGDF receptor, MGMT, MHC(HLA-DR), MIF, MIG, MIP, MIP-1-alpha, MK, MMAC1, MMP, MMP-1, MMP-10, MMP-11, MMP-12, MMP-13, MMP-14, MMP-15, MMP-2, MMP-24, MMP-3, MMP-7, MMP-8, MMP-9, MPIF, Mpo, MSK, MSP, mucin (Muc1), MUC18, Muellerian-inhibitin substance, Mug, MuSK, NAIP, NAP, NAV 1.7, NCAD, N-Cadherin, NCA 90, NCAM, NCAM, Neprilysin, Neurotrophin-3, -4, or -6, Neurturin, Neuronal growth factor (NGF), NGFR, NGF-beta, nNOS, NO, NOS, Npn, NRG-3, NT, NTN, OB, OGG1, OPG, OPN, OSM, OX40L, OX40R, p150, p95, PADPr, Parathyroid hormone, PARC, PARP, PBR, PBSF, PCAD, P-Cadherin, PCNA, PD1, PDL1, PDGF, PDGF, PDK-1, PECAM, PEM, PF4, PGE, PGF, PGI2, PGJ2, PIN, PLA2, placental alkaline phosphatase (PLAP), P1GF, PLP, PP14, Proinsulin, Prorelaxin, Protein C, PS, PSA, PSCA, prostate specific membrane antigen (PSMA), PTEN, PTHrp, Ptk, PTN, R51, RANK, RANKL, RANTES, RANTES, Relaxin A-chain, Relaxin B-chain, renin, respiratory syncytial virus (RSV) F, RSV Fgp, Ret, Rheumatoid factors, RLIP76, RPA2, RSK, S100, SCF/KL, SDF-1, SERINE, Serum albumin, sFRP-3, Shh, SIGIRR, SK-1, SLAM, SLPI, SMAC, SMDF, SMOH, SOD, SPARC, Stat, STEAP, STEAP-II, TACE, TACI, TAG-72 (tumor-associated glycoprotein-72), TARC, TCA-3, T-cell receptors (e.g., T-cell receptor alpha/beta), TdT, TECK, TEM1, TEM5, TEM7, TEM8, TERT, testicular PLAP-like alkaline phosphatase, TfR, TGF, TGF-alpha, TGF-beta, TGF-beta Pan Specific, TGF-beta R1 (ALK-5), TGF-beta R11, TGF-beta RIIb, TGF-beta RIII, TGF-beta1, TGF-beta2, TGF-beta3, TGF-beta4, TGF-beta5, Thrombin, Thymus Ck-1, Thyroid stimulating hormone, Tie, TIMP, TIQ, Tissue Factor, TMEFF2, Tmpo, TMPRSS2, TNF, TNF-alpha, TNF-alpha beta, TNF-beta2, TNFc, TNF-RI, TNF-RII, TNFRSF10A (TRAIL R1Apo-2, DR4), TNFRSF10B (TRAIL R2DR5, KILLER, TRICK-2A, TRICK-B), TNFRSF10C (TRAIL R3DcR1, LIT, TRID), TNFRSF10D (TRAIL R4 DcR2, TRUNDD), TNFRSF11A (RANK ODF R, TRANCE R), TNFRSF11B (OPG OCIF, TR1), TNFRSF12 (TWEAK R FN14), TNFRSF13B (TACI), TNFRSF13C (BAFF R), TNFRSF14 (HVEM ATAR, HveA, LIGHT R, TR2), TNFRSF16 (NGFR p75NTR), TNFRSF17 (BCMA), TNFRSF18 (GITR AITR), TNFRSF19 (TROY TAJ, TRADE), TNFRSF19L (RELT), TNFRSF1A (TNF R1CD120a, p55-60), TNFRSF1B (TNF RII CD120b, p75-80), TNFRSF26 (TNFRH3), TNFRSF3 (LTbR TNF RIII, TNFC R), TNFRSF4 (OX40 ACT35, TXGP1 R), TNFRSF5 (CD40 p50), TNFRSF6 (Fas Apo-1, APT1, CD95), TNFRSF6B (DcR3M68, TR6), TNFRSF7 (CD27), TNFRSF8 (CD30), TNFRSF9 (4-11B CD137, ILA), TNFRSF21 (DR6), TNFRSF22 (DCTRAIL R2 TNFRH2), TNFRST23 (DCTRAIL RITNFRH1), TNFRSF25 (DR3Apo-3, LARD, TR-3, TRAMP, WSL-1), TNFSF10 (TRAIL Apo-2 Ligand, TL2), TNFSF11 (TRANCE/RANK Ligand ODF, OPG Ligand), TNFSF12 (TWEAK Apo-3 Ligand, DR3Ligand), TNFSF13 (APRIL TALL2), TNFSF13B (BAFF BLYS, TALL1, THANK, TNFSF20), TNFSF14 (LIGHT HVEM Ligand, LTg), TNFSF15 (TL1A/VEGI), TNFSF18 (GITR Ligand AITR Ligand, TL6), TNFSF1A (TNF-a Conectin, DIF, TNFSF2), TNFSF1B (TNF-b LTa, TNFSF1), TNFSF3 (LTb TNFC, p33), TNFSF4 (OX40 Ligand gp34, TXGP1), TNFSF5 (CD40 Ligand CD154, gp39, HIGM1, IMD3, TRAP), TNFSF6 (Fas Ligand Apo-1 Ligand, APT1 Ligand), TNFSF7 (CD27 Ligand CD70), TNFSF8 (CD30 Ligand CD153), TNFSF9 (4-1BB Ligand CD137 Ligand), TP-1, t-PA, Tpo, TRAIL, TRAIL R, TRAIL-R1, TRAIL-R2, TRANCE, transferring receptor, TRF, Trk (e.g., TrkA), TROP-2, TSG, TSLP, tumor-associated antigen CA 125, tumor-associated antigen expressing Lewis Y related carbohydrate, TWEAK, TXB2, Ung, UPAR, uPAR-1, Urokinase, VCAM, VCAM-1, VECAD, VE-Cadherin, VE-cadherin-2, VEFGR-1 (fit-1), VEGF, VEGFR, VEGFR-3 (flt-4), VEGI, VIM, Viral antigens, VLA, VLA-1, VLA-4, VNR integrin, von Willebrands factor, WIF-1, WNT1, WNT2, WNT2B/13, WNT3, WNT3A, WNT4, WNT5A, WNT5B, WNT6, WNT7A, WNT7B, WNT8A, WNT8B, WNT9A, WNT9A, WNT9B, WNT10A, WNT10B, WNT11, WNT16, XCL1, XCL2, XCR1, XCR1, XEDAR, XIAP, XPD, and receptors for hormones and growth factor.

In some embodiments, the binding domain specifically binds to one or more therapeutic targets or antigens in the livestock animal, such as, but are not limited to, ACE, ACE-2, Activin, Activin A, Activin AB, Activin B, Activin C, Activin RIA, Activin RIA ALK-2, Activin RIB ALK-4, Activin RIIA, Activin RIIB, ADAM, ADAM10, ADAM12, ADAM15, ADAM17/TACE, ADAMS, ADAM9, ADAMTS, ADAMTS4, ADAMTS5, ANG, Ang, Angiotensin type 1 (AT1) receptor, Angiotensin type 2 (AT2) receptor, Atrial natriuretic factor, av/b3 integrin, b-ECGF, CD19, CD20, CD30, CD34, CD40, CD40L, CD47, COX, CTLA-4, EGFR (ErbB-1), EPO, Follicle stimulating hormone, GDF-8 (Myostatin), GLP1, GLP2, GnRH, Growth hormone releasing factor, IgE, IL, IL-1, IL-1R, IL-2, IL-2R, IL-4, IL-4R, IL-5, IL-5R, IL-6, IL-6R, IL-8, IL-9, IL-10, IL-12, IL-13, IL-15, IL-17, IL-18, IL-18R, IL-21, IL-22, IL-23, IL-25, IL-31, IL-33, interleukin receptor (e.g., IL-1R, IL-2R, IL-4R, IL-5R, IL-6R, IL-8R, IL-9R, IL-10R, IL-12R, IL-13R, IL-15R, IL-17R, IL-18R, IL-21R, IL-22R, IL-23R, IL-25R, IL-31R, IL-33R), LAP (TGF-1), Latent TGF-1, Latent TGF-1 bp1, LFA-1, Neuronal growth factor (NGF), NGFR, NGF-beta, OX40L, OX40R, PD1, PDL1, TGF, TGF-alpha, TGF-beta, TGF-beta Pan Specific, TGF-beta R1 (ALK-5), TGF-beta R11, TGF-beta RIIb, TGF-beta RIII, TGF-beta1, TGF-beta2, TGF-beta3, TGF-beta4, TGF-beta5, TNF, TNF-alpha, TNF-alpha beta, TNF-beta2, TNFc, TNF-RI, TNF-RII, TNFRSF16 (NGFR p75NTR), TNFRSF9 (4-1BB CD137, ILA), VEFGR-1 (fit-1), VEGF, VEGFR, and VEGFR-3 (flt-4).

In some embodiments, the polypeptide or polypeptides can comprise a protein, wherein the protein is a therapeutic protein, e.g., EPO, CTLA4, LFA3, VEGFR1/VEGFR3, IL-1R, IL-4R, GLP-1 receptor agonist, or Thrombopoietin binding peptide. In some embodiments, the therapeutic protein is ACE, ACE-2, Activin, Activin A, Activin AB, Activin B, Activin C, Activin RIA, Activin RIA ALK-2, Activin RIB ALK-4, Activin RIIA, Activin RIIB, ADAM, ADAM10, ADAM12, ADAM15, ADAM17/TACE, ADAMS, ADAM9, ADAMTS, ADAMTS4, ADAMTS5, ANG, Ang, Angiotensin type 1 (AT1) receptor, Angiotensin type 2 (AT2) receptor, Atrial natriuretic factor, av/b3 integrin, b-ECGF, CD19, CD20, CD30, CD34, CD40, CD40L, CD47, COX, CTLA-4, EGFR (ErbB-1), EPO, Follicle stimulating hormone, GDF-8 (Myostatin), GLP1, GLP2, GnRH, Growth hormone releasing factor, IgE, IL, IL-1, IL-1R, IL-2, IL-2R, IL-4, IL-4R, IL-5, IL-5R, IL-6, IL-6R, IL-8, IL-9, IL-10, IL-12, IL-13, IL-15, IL-17, IL-18, IL-18R, IL-21, IL-22, IL-23, IL-25, IL-31, IL-33, interleukin receptor (e.g., IL-1R, IL-2R, IL-4R, IL-5R, IL-6R, IL-8R, IL-9R, IL-10R, IL-12R, IL-13R, IL-15R, IL-17R, IL-18R, IL-21R, IL-22R, IL-23R, IL-25R, IL-31R, IL-33R), LAP (TGF-1), Latent TGF-1, Latent TGF-1 bp1, LFA-1, Neuronal growth factor (NGF), NGFR, NGF-beta, OX40L, OX40R, PD1, PDL1, TGF, TGF-alpha, TGF-beta, TGF-beta Pan Specific, TGF-beta R1 (ALK-5), TGF-beta R11, TGF-beta RIIb, TGF-beta RIII, TGF-beta1, TGF-beta2, TGF-beta3, TGF-beta4, TGF-beta5, TNF, TNF-alpha, TNF-alpha beta, TNF-beta2, TNFc, TNF-RI, TNF-RII, TNFRSF16 (NGFR p75NTR), TNFRSF9 (4-1BB CD137, ILA), VEFGR-1 (fit-1), VEGF, VEGFR, or VEGFR-3 (flt-4).

### Pharmaceutical Compositions

To prepare pharmaceutical or sterile compositions of a polypeptide or polypeptides described herein, the polypeptide or polypeptides can be admixed with a pharmaceutically acceptable carrier or excipient. (See, e.g., Remington's Pharmaceutical Sciences and U.S. Pharmacopeia: National Formulary, Mack Publishing Company, Easton, Pa. (1984)).

Formulations of therapeutic and diagnostic agents may be prepared by mixing with acceptable carriers, excipients, or stabilizers in the form of, e.g., lyophilized powders, slurries, aqueous solutions or suspensions (see, e.g., Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.). In some embodiments, the polypeptide or polypeptides of the present invention are diluted to an appropriate concentration in a sodium acetate solution pH 5-6, and NaCl or sucrose is added for tonicity. Additional agents, such as polysorbate 20 or polysorbate 80, may be added to enhance stability.

Toxicity and therapeutic efficacy of the polypeptide compositions, administered alone or in combination with another agent, can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index (LD₅₀/ED₅₀). In particular aspects, a polypeptide or polypeptides exhibiting high therapeutic indices are desirable. The data obtained from these cell culture assays and animal studies can be used in formulating a range of dosage for use in the livestock animal. The dosage of such compounds lies preferably within a range of circulating concentrations that include the ED₅₀ with little or no toxicity. The dosage may vary within this range depending upon the dosage form employed and the route of administration.

The mode of administration can vary. Suitable routes of administration include oral, rectal, transmucosal, intestinal, parenteral; intramuscular, subcutaneous, intradermal, intramedullary, intrathecal, direct intraventricular, intravenous, intraperitoneal, intranasal, intraocular, inhalation, insufflation, topical, cutaneous, transdermal, or intra-arterial. In some embodiments, the polypeptide or polypeptides can be administered by an invasive route such as by injection. In further embodiments, the polypeptide or polypeptides is administered intravenously, subcutaneously, intramuscularly, intraarterially, intratumorally, or by inhalation, aerosol delivery.

The pharmaceutical compositions disclosed herein may also be administered by infusion. Examples of well-known implants and modules form administering pharmaceutical compositions include: U.S. Pat. No. 4,487,603, which discloses an implantable micro-infusion pump for dispensing medication at a controlled rate; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments. Many other such implants, delivery systems, and modules are well known to those skilled in the art.

Alternatively or additionally, one may also administer the polypeptide or polypeptides in a local manner, for example, via injection of the antibody directly into an arthritic joint or pathogen-induced lesion characterized by immunopathology, often in a depot or sustained release formulation. Furthermore, one may administer the polypeptide or polypeptides in a targeted drug delivery system, for example, in a liposome coated with a tissue-specific antibody, targeting, for example, arthritic joint or pathogen-induced lesion characterized by immunopathology. The liposomes will be targeted to and taken up selectively by the afflicted tissue.

The administration regimen depends on several factors, including, without limitation, the age, weight, and physical condition of the livestock animal to be or being treated, the serum or tissue turnover rate of the therapeutic antibody, the level of symptoms, the immunogenicity of the therapeutic polypeptide or polypeptides, and the accessibility of the target cells in the biological matrix. In some implementations, the administration regimen delivers sufficient therapeutic polypeptide or polypeptides to effect improvement in the target disease state, while simultaneously minimizing undesired side effects. Accordingly, the amount of biologic delivered depends in part on the particular therapeutic polypeptide or polypeptides and the severity of the condition being treated. Guidance in selecting appropriate doses of therapeutic antibodies is available (see, e.g., Wawrzynczak Antibody Therapy, Bios Scientific Pub. Ltd, Oxfordshire, U K (1996); Milgrom et al. New Engl. J. Med. 341:1966-1973 (1999); Slamon et al. New Engl. J. Med. 344:783-792 (2001); Beniaminovitz et al. New Engl. J. Med. 342:613-619 (2000); Ghosh et al. New Engl. J. Med. 348:24-32 (2003); Lipsky et al. New Engl. J. Med. 343:1594-1602 (2000)).

Determination of the appropriate dose of the polypeptide or polypeptides is made by one skilled in the art, e.g., using parameters or factors known or suspected in the art to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced.

### Nucleic Acids, Vectors, Host Cells, and Methods of Making

The disclosure also encompasses nucleic acid or nucleic acids encoding the polypeptide or polypeptides described herein, a vector or vectors comprising the nucleic acid or nucleic acids, and host cells comprising the nucleic acid or nucleic acids or the vector or vectors.

The polypeptide or polypeptides described herein may be produced in bacterial or eukaryotic cells. Some polypeptides, e.g., Fab's, can be produced in bacterial cells, e.g., E. coli cells. Polypeptides can also be produced in eukaryotic cells such as transformed cell lines (e.g., CHO, 293E, COS, 293T, Hela). In addition, polypeptides (e.g., scFv's) can be expressed in a yeast cell such as Pichia (see, e.g., Powers et al., J Immunol Methods. 251:123-35 (2001)), Hanseula, or Saccharomyces. To produce the antibody of interest, a polynucleotide or polynucleotides encoding the polypeptide or polypeptides is/are constructed, introduced into an expression vector or expression vectors, and then expressed in suitable host cells. To improve expression, the nucleotide sequences of the genes can be recoded without changing (or minimally changing—e.g., removal of a C-terminal residue of the heavy or light chain) the amino acid sequence. The areas for potential recoding include those associated with translation initiation, codon usage, and possible unintended mRNA splicing. Polynucleotides encoding an Fc region variant described herein would be readily envisioned by the ordinarily skilled artisan.

Standard molecular biology techniques can be used to prepare the recombinant expression vector(s), transfect the host cells, select for transformants, culture the host cells, and recover the polypeptide (e.g., antibody).

If the polypeptide or polypeptides is to be expressed in bacterial cells (e.g., E. coli), the expression vector should have characteristics that permit amplification of the vector in the bacterial cells. Additionally, when E. coli such as JM109, DH5α, HB101, or XL1-Blue is used as a host, the vector must have a promoter, for example, a lacZ promoter (Ward et al., 341:544-546 (1989), araB promoter (Better et al., Science, 240:1041-1043 (1988)), or T7 promoter that can allow efficient expression in E. coli. Examples of such vectors include, for example, M13-series vectors, pUC-series vectors, pBR322, pBluescript, pCR-Script, pGEX-5X-1 (Pharmacia), “QIAEXPRESS® system” (QIAGEN), pEGFP, and pET (when this expression vector is used, the host is preferably BL21 expressing T7 RNA polymerase). The expression vector may contain a signal sequence for antibody secretion. For production into the periplasm of E. coli, the pelB signal sequence (Lei et al., J. Bacteriol., 169:4379 (1987)) may be used as the signal sequence for antibody secretion. For bacterial expression, calcium chloride methods or electroporation methods may be used to introduce the expression vector into the bacterial cell.

If the polypeptide or polypeptides is to be expressed in animal cells such as CHO, COS, and NIH3T3 cells, the expression vector includes a promoter necessary for expression in these cells, for example, an SV40 promoter (Mulligan et al., Nature, 277:108 (1979)) (e.g., early simian virus 40 promoter), MMLV-LTR promoter, EF1α promoter (Mizushima et al., Nucleic Acids Res., 18:5322 (1990)), or CMV promoter (e.g., human cytomegalovirus immediate early promoter). In addition to the nucleic acid sequence encoding the Fc region variant, the recombinant expression vectors may carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216, 4,634,665 and 5,179,017). For example, typically the selectable marker gene confers resistance to drugs, such as G418, hygromycin, or methotrexate, on a host cell into which the vector has been introduced. Examples of vectors with selectable markers include pMAM, pDR2, pBK-RSV, pBK-CMV, pOPRSV, and pOP13.

In some embodiments, the polypeptide or polypeptides are produced in mammalian cells. Exemplary mammalian host cells for expressing polypeptide or polypeptides include Chinese Hamster Ovary (CHO cells) (including dhfr− CHO cells, described in Urlaub and Chasin (1980) Proc. Natl. Acad. Sci. USA 77:4216-4220, used with a DHFR selectable marker, e.g., as described in Kaufman and Sharp (1982) Mol. Biol. 159:601 621), human embryonic kidney 293 cells (e.g., 293, 293E, 293T), COS cells, NIH3T3 cells, lymphocytic cell lines, e.g., NS0 myeloma cells and SP2 cells, and a cell from a transgenic animal, e.g., a transgenic mammal. For example, the cell is a mammary epithelial cell.

In an exemplary system for antibody expression, a recombinant expression vector encoding both the antibody heavy chain and the antibody light chain of the antibody is introduced into dhfr− CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and the antibody is recovered from the culture medium.

### Methods of Treatment

The polypeptide or polypeptides disclosed herein can be used to treat or prevent any disease or disorder in a livestock animal in need thereof. This invention is particularly helpful in the treatment of chronic conditions where repeated dosing is required. Because of the increased half-life of the protein therapeutic, less frequent dosing and/or reduced dose levels may be possible.

In some embodiments, the disease, disorder, condition or symptoms being treated or prevented is an allergic disease, a chronic pain, an acute pain, an inflammatory disease, an autoimmune disease, an endocrine disease, a gastrointestinal disease, a skeletal/musculoskeletal disease, a cardiovascular disease, a neurological disease, a renal disease, a metabolic disease, a immunological disease, a genetic/inherited disease, a fertility related disorder, an infectious disease or a cancer. In certain embodiments, the disease or disorder being treated or prevented is atopic dermatitis, allergic dermatitis, food allergy, osteoarthritic pain, perioperative pain, dental pain, cancer pain, arthritis, anemia, obesity, or diabetes.

Antibodies may not only be used to treat or prevent disease but also to modulate normal biological function, for example, to manage fertility or behavior.

### Diagnosis

The polypeptide or polypeptides disclosed herein can also be used for various diagnostic purposes, for example, to determine whether the livestock animal has any particular disease or disorder. In some embodiments, the polypeptide or polypeptides may comprise a binding domain. The binding domain can specifically bind to a protein, subunit, domain, motif, and/or epitope as described herein (e.g., a maker for cancer cells). In some embodiments, the polypeptide or polypeptides further comprises a labeling group. In general, label groups fall into a variety of classes, depending on the assay in which they are to be detected: a) isotopic labels, which may be radioactive or heavy isotopes; b) magnetic labels (e.g., magnetic particles); c) redox active moieties; d) optical dyes; enzymatic groups (e.g. horseradish peroxidase, β-galactosidase, luciferase, alkaline phosphatase); e) biotinylated groups; and f) predetermined polypeptide epitopes recognized by a secondary reporter (e.g., leucine zipper pair sequences, binding sites for secondary antibodies, metal binding domains, epitope tags, etc.). In some embodiments, the labelling group is coupled to the antibody via spacer arms of various lengths to reduce potential steric hindrance. Various methods for labelling proteins are known in the art and may be used in performing the present invention.

In some embodiments, the labeling group is a probe, a dye (e.g., a fluorescent dye), or a radioactive isotope (e.g., ³H, ¹⁴C, ²²Na, ³⁶Cl, ³⁵S, ³³P, or ¹²⁵I).

Specific labels can also include optical dyes, including, but not limited to, chromophores, phosphors and fluorophores, with the latter being specific in many instances. Fluorophores can be either “small molecule” fluores, or proteinaceous fluores.

The fluorescent label can be any molecule that may be detected via its inherent fluorescent properties. Suitable fluorescent labels include, but are not limited to, fluorescein, rhodamine, tetramethylrhodamine, eosin, erythrosin, coumarin, methyl-coumarins, pyrene, Malachite green, stilbene, Lucifer Yellow, CASCADE BLUEJ®, Texas Red, IAEDANS, EDANS, BODIPY FL, LC Red 640, Cy 5, Cy 5.5, LC Red 705, Oregon green, the ALEXA FLUOR® dyes (ALEXA FLUOR® 350, ALEXA FLUOR® 430, ALEXA FLUOR® 488, ALEXA FLUOR® 546, ALEXA FLUOR® 568, ALEXA FLUOR® 594, ALEXA FLUOR® 633, ALEXA FLUOR® 660, ALEXA FLUOR® 680), CASCADE BLUE®, CASCADE YELLOW® and R-phycoerythrin (PE) (Molecular Probes, Eugene, Oreg.), FITC, Rhodamine, and Texas Red (Pierce, Rockford, Ill.), Cy5, Cy5.5, Cy7 (Amersham Life Science, Pittsburgh, Pa.). Suitable optical dyes, including fluorophores, are described in Molecular Probes Handbook by Richard P. Haugland, which is incorporated by reference in its entirety.

Suitable proteinaceous fluorescent labels also include, but are not limited to, green fluorescent protein, including a Renilla, Ptilosarcus, or Aequorea species of green fluorescent protein (GFP, Chalfie et al., 1994, Science 263:802-805), enhanced GFP (EGFP, Clontech Laboratories, Inc., GENBANK® Accession Number U55762), blue fluorescent protein (BFP, Quantum Biotechnologies, Inc. 1801 de Maisonneuve Blvd. West, 8th Floor, Montreal, Quebec, Canada H3H1J9; Stauber, 1998, Biotechniques 24:462-471; Heim et al., 1996, Curr. Biol. 6:178-182), enhanced yellow fluorescent protein (EYFP, Clontech Laboratories, Inc.), luciferase (Ichiki et al., 1993, J. Immunol. 150:5408-5417), R galactosidase (Nolan et al., 1988, Proc. Natl. Acad. Sci. U.S.A. 85:2603-2607) and Renilla (WO92/15673, WO95/07463, WO98/14605, WO98/26277, WO99/49019, U.S. Pat. Nos. 5,292,658, 5,418,155, 5,683,888, 5,741,668, 5,777,079, 5,804,387, 5,874,304, 5,876,995, 5,925,558). All of the above-cited references in this paragraph are expressly incorporated herein by reference in the entirety.

### Assays

### FcγRI and FcγRIII Binding:

Binding to FcγRI and FcγRIII is a measure of the ability of an antibody to mediate antibody-dependent cellular cytotoxicity (ADCC). In order to assess this property for an antibody an assay to measure binding of the antibody to FcγRI and FcγRIII can be conducted using methods known in the art.

### C1q Binding:

Binding to the first component of complement, C1q, is a measure of the ability of an antibody to mediate complement-dependent cytotoxicity (CDC). In order to assess this property for an antibody, an assay to measure binding of the antibody to C1q can be conducted using methods known in the art.

### Half-Life:

Methods of measuring half-life of an antibody are well known in the art. See, e.g., Booth et al., MAbs, 10(7):1098-1110 (2018). As an example, the half-life of an antibody can be measured by injection of the antibody into the species from which the Fc is derived (target species) and measuring levels of the antibody in the serum over a certain period of time. Alternatively a transgenic mouse model could be used that is null for murine FcRn alpha chain and express the target species FcRn alpha transgene. The target species FcRn alpha chain could either be paired with mouse P2-microglobulin protein or the mouse P2-microglobulin gene could be substituted with the target species P2-microglobulin gene thereby forming a native FcRn alpha chain-β2-microglobulin heterodimer protein.

### EXAMPLES

### Example 1: Generation of NNK Saturation Mutagenesis Libraries at Selected Positions and Analysis of Individual Variants

The wild-type (wt) sequences of the CH2 and CH3 domains of porcine, bovine, equine, ovine and caprine IgG (as shown in SEQ ID NOs:1 to 27) are synthesized and used as templates for NNK mutagenesis. The NNK saturation mutagenesis method is an effective strategy to generate all 20 possible amino acids at a desired position (Hogrefe et al., Biotechniques. 33: 1158-1165 [2002]; the content of which is incorporated herein by reference in its entirety). Individual NNK libraries at positions 250, 252, 254, 256, 286, 309, 311, 426, 428, 434 and 436 (EU numbering) are generated. NNK (N=A/C/G/T, K=G/T) primers at the specified position are used with the QuikChange Site-Directed Mutagenesis Kit (Agilent). The PCR-product is subcloned into the GenScript FASEBA plasmid, transformed into E. coli and sequenced verified for the presence of the variant. Downstream of the CH2 domain is the SASA (single-domain antibody against serum albumin) tag (Zhang, J.; Wu, S.; Liu, J. Methods and systems for increasing protein stability. Patent application no: US 2013/0129727 A1; the content of which is incorporated herein by reference in its entirety), which has pM affinity for albumin. The SASA antibody enables the capture of the Fc to the sensor chip surface described below. The PelB (pectate lyase B) signal peptide is at the N-terminus to facilitate secretion of the Fc into the medium. The expression of CH2-CH3 protein is regulated by the Lac promoter. The supernatants from conditioned medium are analyzed for binding to the FcRn of the target livestock animal/species at pH 6.0 for variants using surface plasmon resonance (SPR).

The supernatants from multiple individual transformants from each library are assayed for binding to the FcRn of the livestock animal/species at pH 6.0 using the BIACORE™ method, as described below.

For the SPR analyses using the BIACORE™ 8K, bovine serum albumin (BSA) is immobilized to CM5 sensor chip. The sensor chip surface of flow cells 1 and 2 are activated by freshly mixed 50 mmol/L N-Hydroxysuccinimide and 200 mmol/L 1-ethyl-3-(3-dimethylaminopropyl) carbodiimide hydrochloride for 420s (10 μL/min). Afterwards, BSA diluted in 10 mM sodium acetate (pH 4.5) is injected into the flow cell 2 to achieve conjugation, while flow cell 1 is set as blank. After the amine coupling reaction, the remaining active coupling sites on chip surface are blocked with 420 s injection of 1 mM ethanolamine hydrochloride. The running buffer for the binding experiment is HBS-EP (10 mM HEPES, 500 mM NaCl, 3 mM EDTA, 0.05% TWEEN® 20, pH 5.5) and run at 25° C. Supernatants from the variants are injected over chip surface and captured via the SASA tag onto the immobilized BSA for 60 sec. FcRn of the livestock animal/species at 200 nM is injected for 120 sec and the dissociation is complete with running buffer for 120 sec. The flow rate for the immobilization phase of BSA is 10 μL/min and the flow rate for the association and dissociation phase is 30 μL/min. All of the data is processed using the BIACORE™ 8K evaluation software version 1.1. The results will show that the IgG Fc variants tested possess increased binding affinity for an FcRn of the corresponding livestock animal/species at pH 6.0 when compared to wild type IgG Fc of the same livestock animal/species.

### Example 2: Scanning Mutagenesis of IgG Fc

A phage display library approach is used to identify IgG1 Fc variants that possess increased affinity to an FcRn of the same livestock animal or species at pH 6.0. The IgG Fc (comprising the CH2+CH3 domains of SEQ ID NO: 1-27) are synthesized by Twist Bioscience to produce multiple variants with amino acid mutations (substitutions) at one or more positions. At each of the mutated positions, eight possible amino acids are substituted. These amino acids are arginine and lysine (positively charged side chain), aspartic acid and glutamic acid (negatively charged side chain), threonine and glutamine (polar uncharged side chain), and leucine and valine (hydrophobic side chain). The Fc DNA library is designed to have an average of two variants per Fc molecule. The number of combinations of modifications can be calculated using the formula: nCr=n!/r!*(n−r)!, where n represents the number of sites, and r represents the number of variants per molecule. The Fc variants with the desired site-specific mutations are printed as mutagenic oligonucleotides on Twist's silicon-based platform.

The oligonucleotides are then assembled to create a full-length Fc gene fragment pool using assembly PCR. The assembled Fc gene fragment pool is then cloned into the pADL-22c phagemid vector from Antibody Design Labs into the Sfi cut-sites. The cloned DNA library is transformed into electrocompetent TG1 E. coli cells to create an experimental diversity of typically >10¹⁰ variants. The phagemid transformed E. coli cells are then co-transfected with M13K07 helper phage to generate a phage pool that is used for protein-based panning. The library is resuspended into 20 mM MES buffer, pH 6.0, 0.05% TWEEN® 20 and 3% milk.

The quality of the library is determined by picking 96 random phage clones and sequenced by the Sanger method.

For the first phage selection, a Protein A capture step is used to eliminate any Fc variants that have lost Protein A binding. For this selection, the phage library is captured onto Protein A beads and washed with phosphate-buffered saline (PBS), pH 7.4. The phage are eluted with 0.1 M glycine, pH 2.7 and the pH is immediately neutralized with 1 M Tris-HCl, pH 7.5. The neutralized phage is precipitated with polyethylene glycol/NaCl and centrifuged. The pelleted phage are resuspended in 20 mM MES, pH 6.0, 0.05% TWEEN® 20, 3% milk.

The next phage selections are based on the protocol described by Borrok et al., 2015, J Biol. Chem., 290:4282, the content of which is incorporated herein by reference in its entirety. Briefly, NUNC™ 96 multi-well plates are coated with Neutravidin and then blocked with 5% bovine serum albumin, PBS, pH 7.4. Biotinylated FcRn of the target livestock animal or species is immobilized in the well at a concentration of about 0.30 μg/ml in PBS, pH 6.0. The phage library in PBS, pH 6.0 is incubated with the immobilized FcRn and then washed with PBS, pH 6.0, 0.05% TWEEN® 20, 0.3 M NaCl. The phage is eluted with PBS, pH 7.4 by incubating at 37° C. for 30 minutes. The eluted phage are depleted with about 0.30 μg/ml of the FcRn of the same livestock animal or species at pH 7.4. The unbound phage is amplified in TG1 cells.

The clones are then sequenced by next generation sequencing using the Illumina MISEQ™.

Unique variants may be reformatted into IgG and mini-prep plasmid DNA transfected into EXPI293™ cells with EXPIFECTAMINE™ 293 transfection reagent. The Ig variants can then be purified from the conditioned medium with Protein A chromatography and formulated into 43 mM sodium citrate, 130 mM sodium bicarbonate, pH 6.0.

For determining the affinities of the IgG variants to the FcRn of the corresponding livestock animal/species, a CARTERRA® instrument can be used to determine the binding kinetics. With this approach, the antibodies (˜5 μg/ml) are typically amine-coupled to the HC30M sensor chip by EDC/NHS activation, followed by ethanolamine HCl quenching.

Different concentrations (333 nM, 111 nM, 37 nM, 12.3 nM, 4.1 nM, 1.37 nM, 0.45 nM) of FcRn is flowed over the sensor chip in HBSTE (10 mM HEPES, 150 mM NaCl, 3 mM EDTA, 0.05% TWEEN® 20), 0.5% bovine serum albumin, pH 6.0 to determine the kinetics at pH 6.0. This same strategy can be used to determine the binding kinetics to FcRn at pH 7.4 except the pH of the HBSTE buffer is adjusted to 7.4.

A list of the amino acid substitutions that increase the binding of the IgG Fc variant to their corresponding FcRN of the same livestock animal/species is provided in Table 6, below:

TABLE 6
Summary of the amino acid substitutions for livestock
animal IgG Fc variants that increase binding affinity
to FcRn of the corresponding livestock animal

| EU | Pig | Cattle | Horse | Sheep | Goat | Amino acid |
|---|---|---|---|---|---|---|
| Number | [11] | [3] | [7] | [3] | [3] | substitution(s) |
| 250 | T, I | T | T, A, V | T, S | T, S | Q, E |
| 252 | M | T, M | M, K | M, T | T, M | Y, W, F |
| 254 | S | S, T | T, S | S, T | S, T | T, R, K, H * |
| 256 | T | T | T, M, K | T | T, K | A, D, E |
| 286 | T | T | T, S | T | T | Y, F, W, L, |
| | | | | | | D, E |
| 309 | Q | Q | Q | Q | Q | D, E, V |
| 311 | Q, E | Q | Q, K | Q, D | Q, D, K | V, K, R, L, |
| | | | | | | H ** |
| 426 | A, V | V, A | G, A, V | V | V, A | Y, F, H |
| 428 | M, H | M | M | M | M | L, Y, F *** |
| 434 | N, H | N | N, H | N | N | A, H, F, S, |
| | | | | | | W, Y |
| 436 | Y, T | Y | Y, F, V | Y | Y | H |

[n] denotes the number of different IgG subclasses identified in that species
* not T at that position for ovine or caprine IgG Fc
** not K at this position for equine IgG4 Fc and IgG7 Fc
*** no substitution at this position for bovine IgG Fc

### Example 3: Binding Kinetics of IgG Fc Variants to FcRn

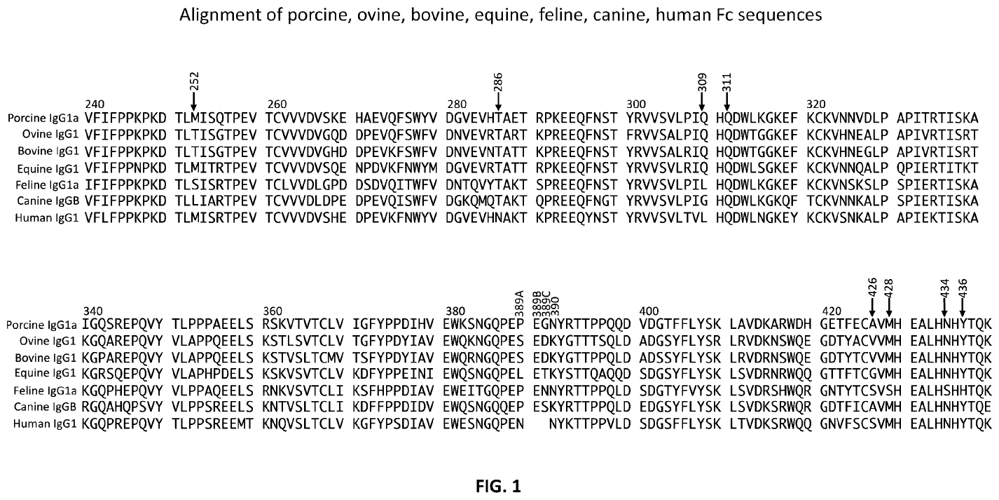

A set of Fc variants was expressed and purified for porcine, ovine, bovine, and equine IgGs. The variable domain for the heavy and light chain for each of the IgGs was previously described by Gearing et al. (2016, J Vet Intern Med, 30:1129). The heavy and light chain constant domains used for the different species' IgGs were as follows: for porcine it was IgG1a (GENBANK® AAA52219.1 SEQ ID NO: 1) and the porcine kappa chain (GENBANK® AHB17990.1), for ovine it was IgG1 (GENBANK® X69797, SEQ ID NO: 22) and the ovine kappa chain (GENBANK® AU45094.1), for bovine it was IgG1 (GENBANK® ABE68619.1, SEQ ID NO: 12) and the bovine kappa chain (GENBANK® AEM45004.1), and for equine it was IgG1 (GENBANK® CAC44760.1, SEQ ID NO: 15) and the equine kappa chain (GENBANK® CAA53284.1). The variants were made in the Fc of each species at positions 252, 286, 309, 311, 426, 428, 434, and 436 (FIG. 1) (EU numbering). The synthesized DNAs for each variant and wild-type were subcloned into PCDNA™ 3.4 expression vector (Life Technologies) and expressed in HEK293 cells. The conditioned medium for each variant and wild-type were purified with the resins shown in Table 7 and the purified antibodies were formulated in PBS, pH 7.2. All of the resins were acquired from Cytiva with the exception of ROBOCOLUMN ESHMUNO® A which was purchased from EMD Millipore.

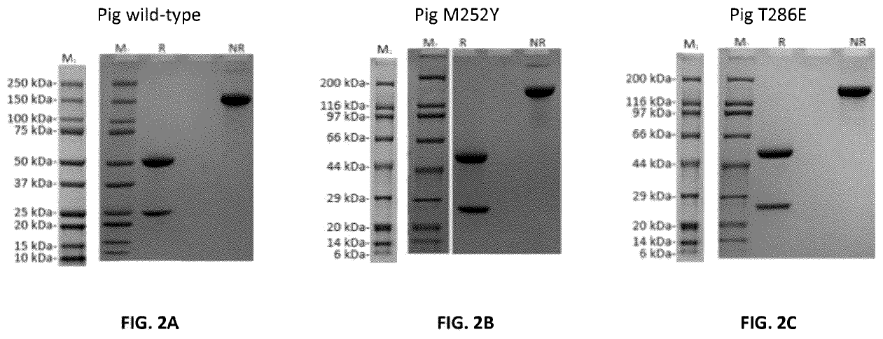

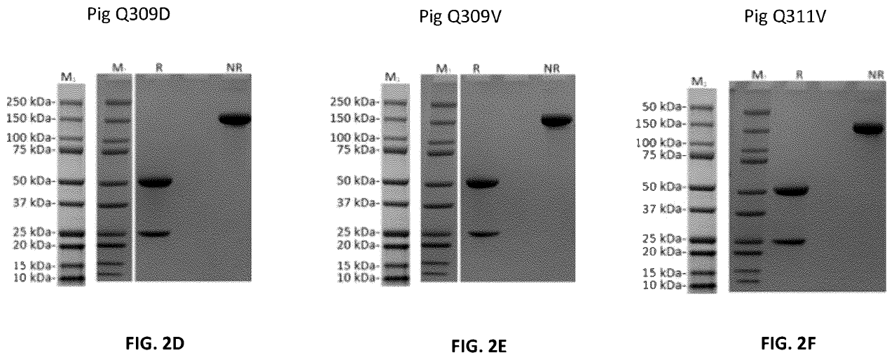

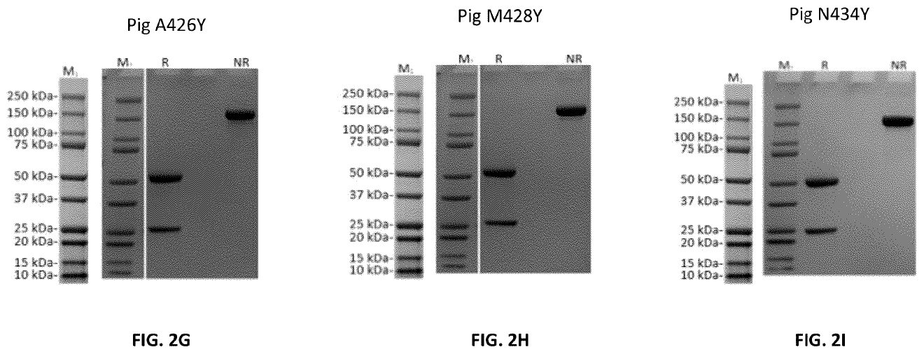

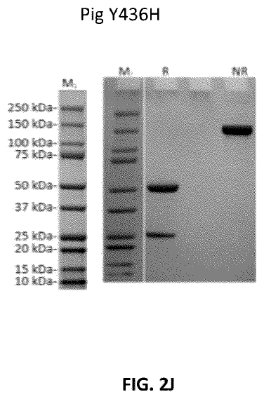

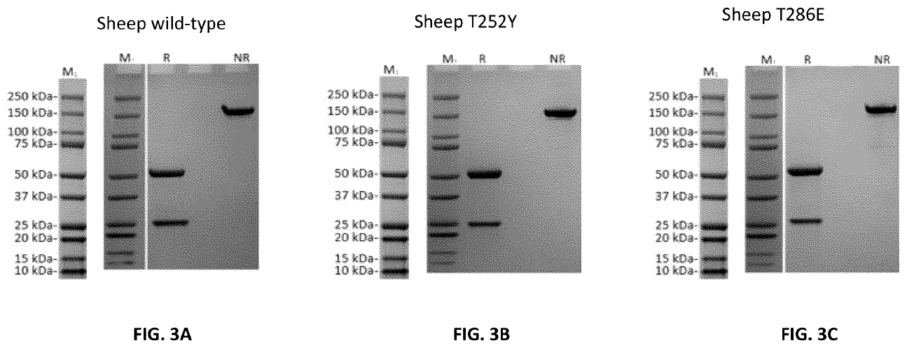

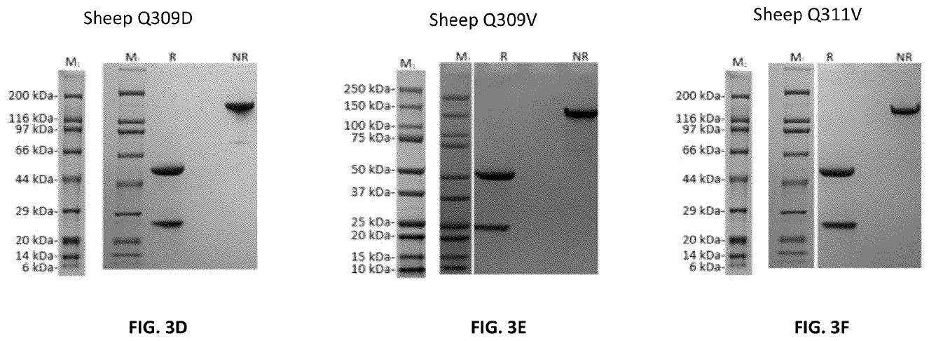

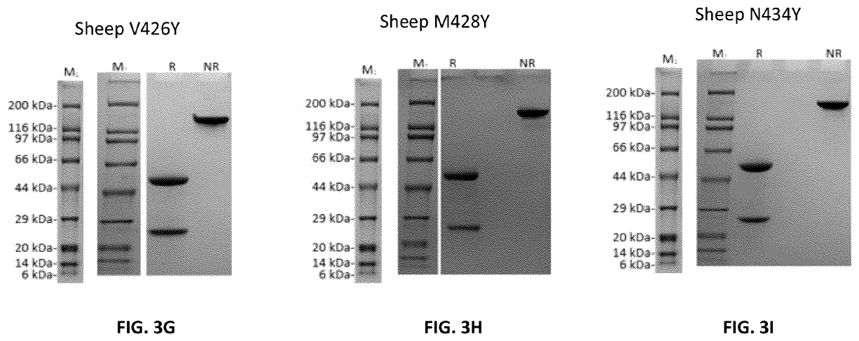

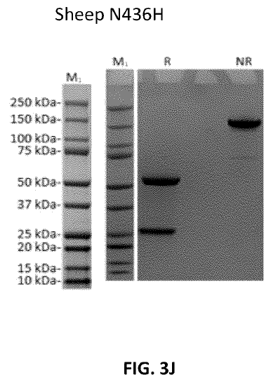

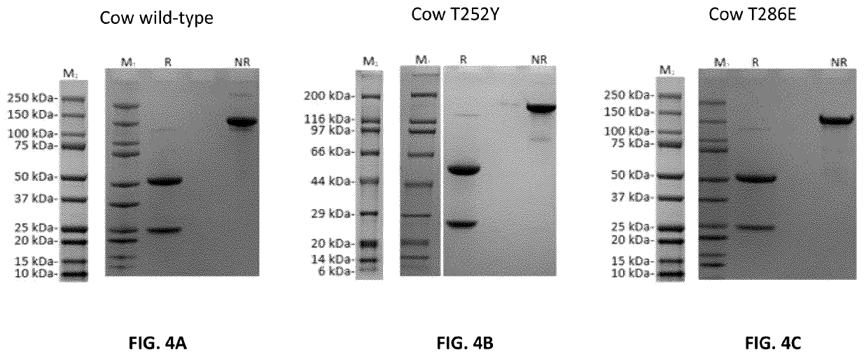

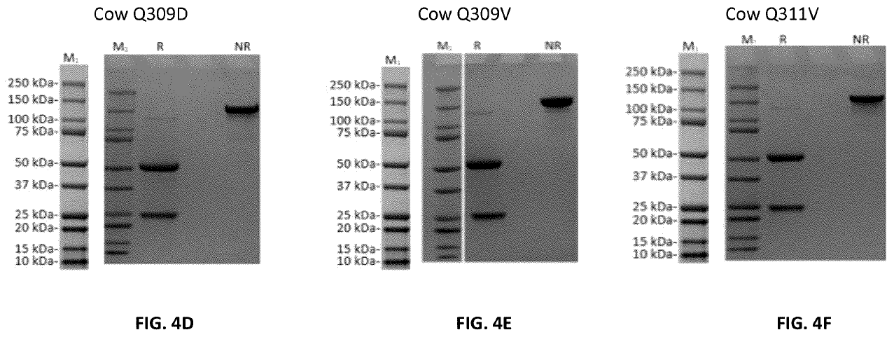

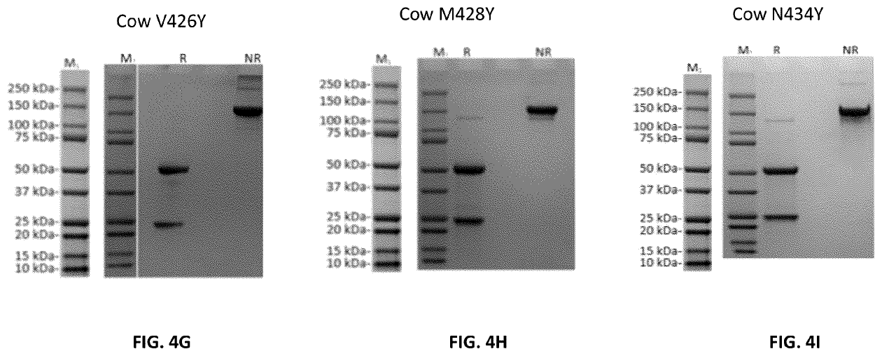

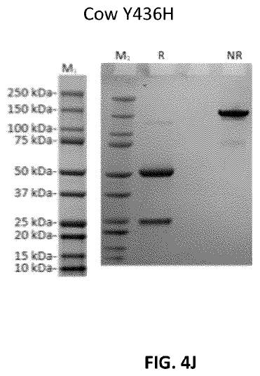

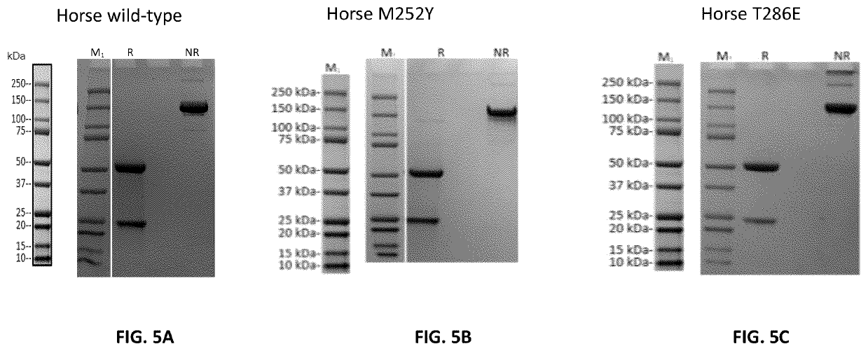

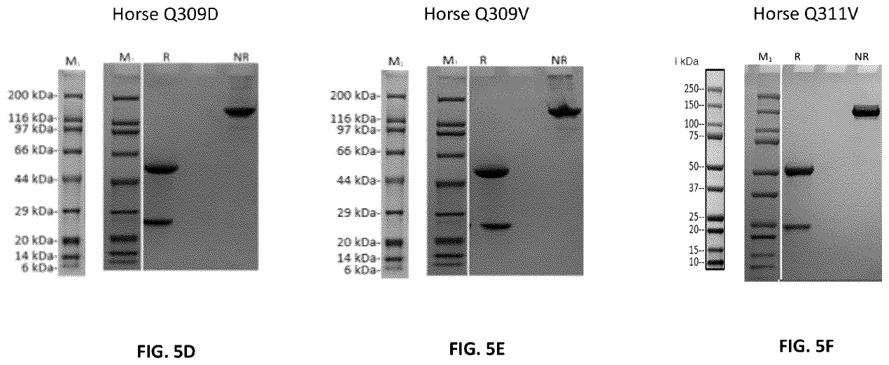

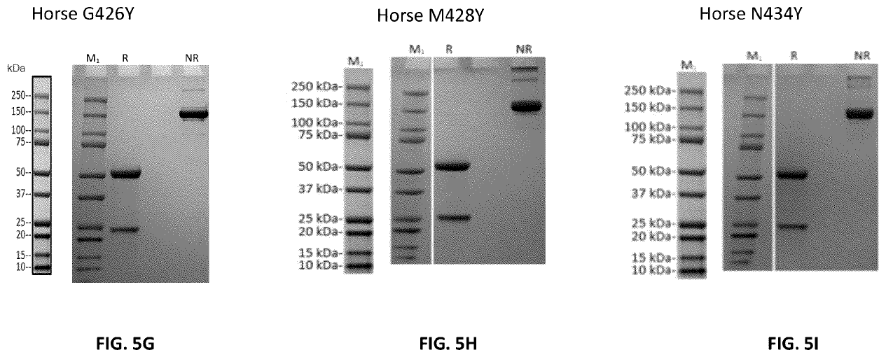

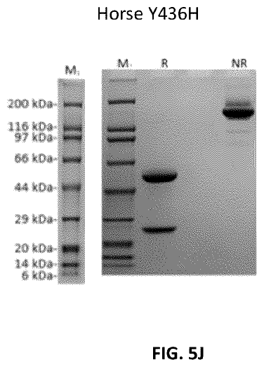

The purity of the antibodies was determined by scanning densitometry of Coomassie blue-stained SDS/PAGE gels and each was >90% (FIGS. 2A-2J, 3A-3J, 4A-4J, 5A-5J).

TABLE 7
Resins for purification of IgG Fc variants and wild-type IgG

| Name | Purification |
|---|---|
| Pig wild-type | PreDictor RoboColumn MabSelect Sure |
| Pig M252Y | PreDictor RoboColumn MabSelect Sure |
| Pig T286E | RoboColumn Eshmuno A 0.6 ml |
| Pig Q309D | PreDictor RoboColumn MabSelect Sure |
| Pig Q309V | PreDictor RoboColumn MabSelect Sure |
| Pig Q311V | PreDictor RoboColumn MabSelect Sure |
| Pig A426Y | PreDictor RoboColumn MabSelect Sure |
| Pig M428Y | PreDictor RoboColumn MabSelect Sure |
| Pig N434Y | PreDictor RoboColumn MabSelect Sure |
| Pig Y436H | PreDictor RoboColumn MabSelect Sure |
| Sheep wild-type | PreDictor RoboColumn MabSelect Sure |
| Sheep T252Y | PreDictor RoboColumn MabSelect Sure |
| Sheep T286E | HiTrap Protein G HP |
| Sheep Q309D | PreDictor RoboColumn MabSelect Sure |
| Sheep Q309V | HiTrap Protein G HP |
| Sheep Q311V | PreDictor RoboColumn MabSelect Sure |
| Sheep V426Y | PreDictor RoboColumn MabSelect Sure |
| Sheep M428Y | RoboColumn Eshmuno A 0.6 ml |
| Sheep N434Y | RoboColumn Eshmuno A 0.6 ml |
| Sheep N436H | HiTrap Protein G HP |
| Cow wild-type | PreDictor RoboColumn MabSelect Sure |
| Cow T252Y | RoboColumn Eshmuno A 0.6 ml |
| Cow T286E | PreDictor RoboColumn MabSelect Sure |
| Cow Q309D | HiTrap Protein G HP |
| Cow Q309V | RoboColumn Eshmuno A 0.6 ml |
| Cow Q311V | RoboColumn Eshmuno A 0.6 ml |
| Cow V426Y | RoboColumn Eshmuno A 0.6 ml |
| Cow M428Y | RoboColumn Eshmuno A 0.6 ml |
| Cow N434Y | RoboColumn Eshmuno A 0.6 ml |
| Cow Y436H | RoboColumn Eshmuno A 0.6 ml |
| Horse wild-type | RoboColumn Eshmuno A 0.6 ml |
| Horse M252Y | RoboColumn Eshmuno A 0.6 ml |
| Horse T286E | RoboColumn Eshmuno A 0.6 ml |
| Horse Q309D | RoboColumn Eshmuno A 0.6 ml |
| Horse Q309V | RoboColumn Eshmuno A 0.6 ml |
| Horse Q311V | RoboColumn Eshmuno A 0.6 ml |
| Horse G426Y | RoboColumn Eshmuno A 0.6 ml |
| Horse M428Y | RoboColumn Eshmuno A 0.6 ml |
| Horse N434Y | RoboColumn Eshmuno A 0.6 ml |
| Horse Y436H | RoboColumn Eshmuno A 0.6 ml |

The FcRn complex consists of a large subunit p51 and beta-2-microglobulin. The Avi-tag sequence (SEQ ID NO: 28, GLNDIFEAQKIEWHE) and an 8× His tag was fused to the C-terminus of the extracellular domain of the large subunit p51 (porcine: GENBANK® E9LK24; ovine: GENBANK® Q8HZV2; bovine: GENBANK® AAF60956.1; equine: GENBANK® XP_023505908.1) and subcloned into PCDNA™ 3.4 expression vector (Life Technologies). The FLAG™ tag (SEQ ID NO: 29, DYKDDDDK) was fused to the C-terminus of the beta-2-microglobulin (porcine: GENBANK® L13854.1; ovine: GENBANK® AY549962.1; bovine: GENBANK® X69084.1; equine: GENBANK® X69083.1) and subcloned into the PCDNA™ 3.4 expression vector. For each species, the large subunit p51 expression vector was co-transfected with the beta-2-microglobulin into HEK293 cells. The FcRn complexes were purified from conditioned medium using HISTRAP™ fast flow chromatography (Cytiva) and the proteins were then formulated into PBS, pH 7.2. The proteins were analyzed by SDS/PAGE and analytical SEC (size exclusion chromatography) using a TSKGEL® G3000SWxl column. The purity of the proteins was >95% by SDS/PAGE and >95% of the FcRn complex was a heterodimer of the large subunit p51 and beta-2-microglobulin by analytical SEC.

The FcRn binding experiments were completed on a BIACORE™ 8K+ system at 25° C. A polyclonal antibody against the His tag was coupled to the CM5 biosensor chip using the His Capture Kit (Cytiva, Cat 28995056) following the manufacturer's instructions. The His-tagged FcRn from the different species were flowed on the anti-His polyclonal biosensor chip at 10 μl/min until approximately 100 RU were captured. The different porcine, ovine, bovine, and equine IgG variants and wild-type were the analytes for the pig, cow, horse, and sheep FcRn proteins biosensor chips, respectively. Five concentrations of IgGs were injected for 90 seconds at 30 μl/min with a 30 second dissociation time. For the wild-type IgGs of each species, the concentrations of IgG were 6.2 nM, 18 nM, 55 nM, 167 nM, and 500 nM. For the variant IgGs of each species, the concentrations of IgG were 1.2 nM, 3.7 nM, 11 nM, 33 nM, and 100 nM. The running buffer was 1×PBS-P+ (Cytiva, Cat #28995084) adjusted to pH 5.9. The surface was regenerated with 10 mM glycine, pH 1.5 at 30 μl/min for 30 seconds. Data were evaluated using Insight Evaluation Software by fitting to a 1:1 kinetic interaction model, or by fitting to steady state affinity.

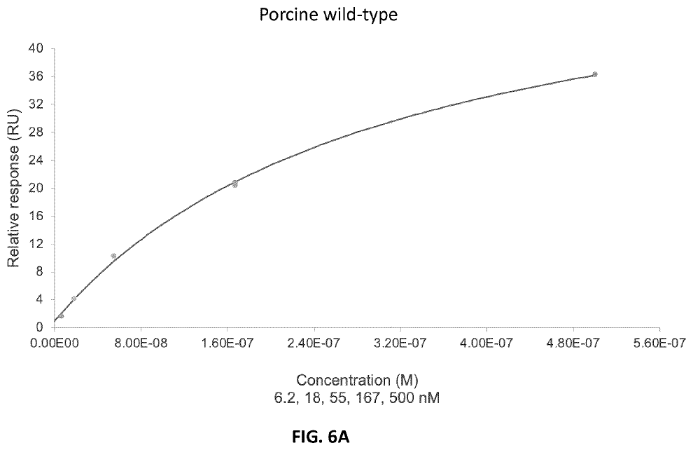

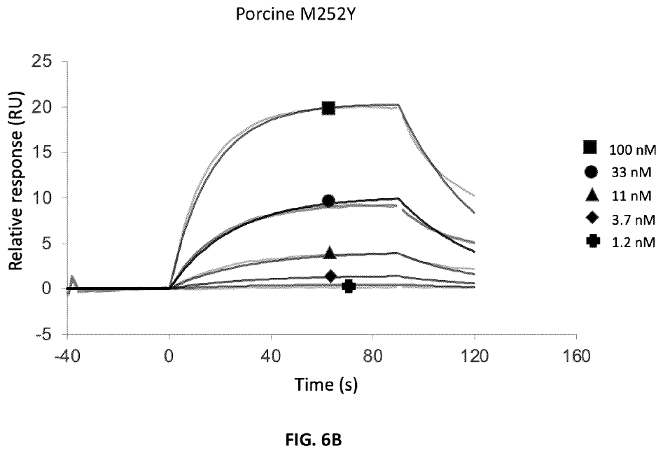

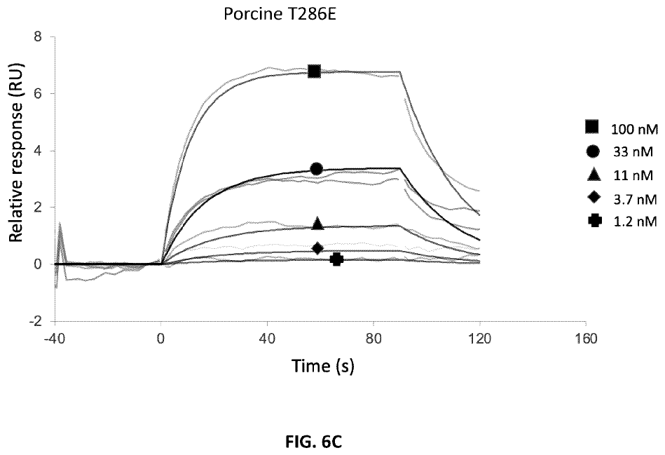

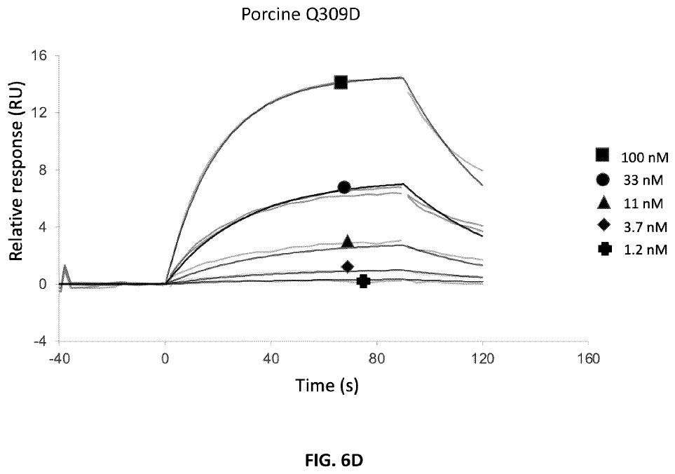

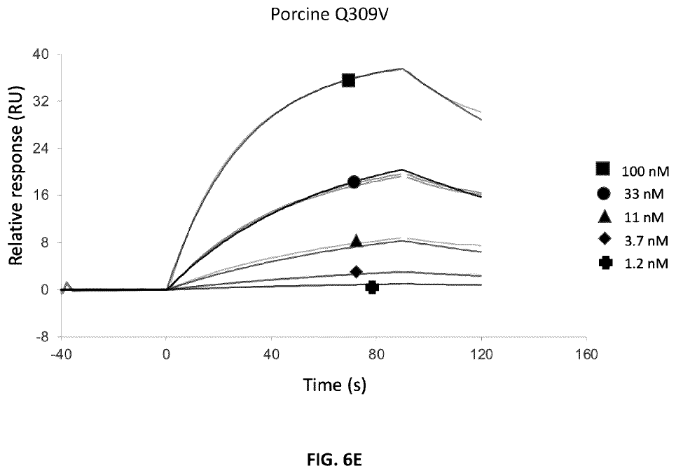

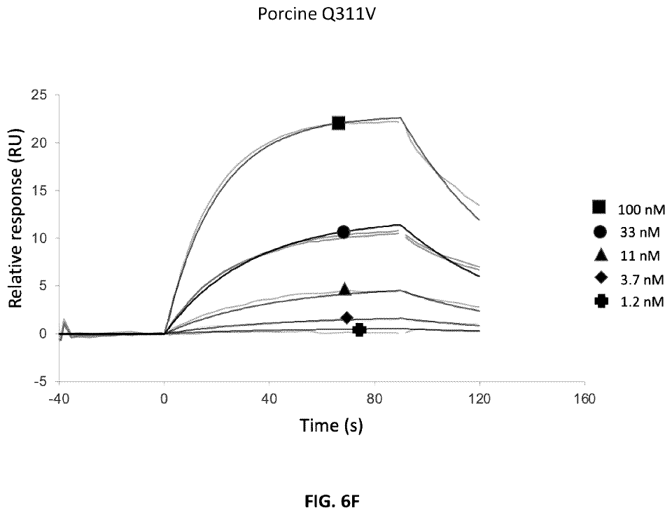

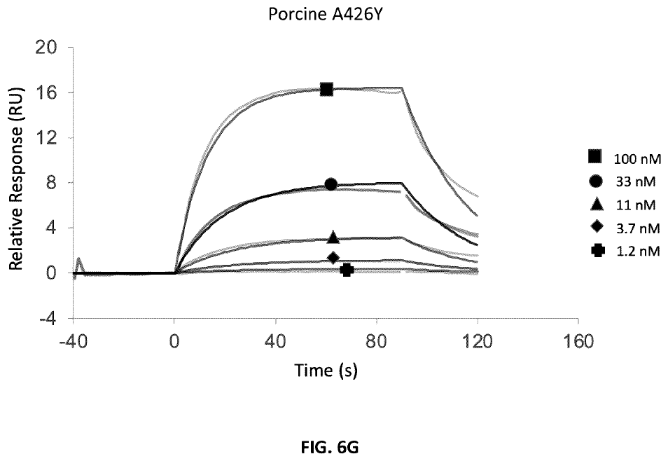

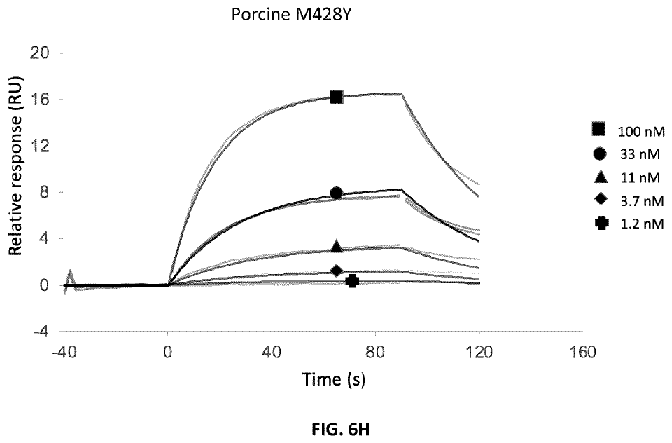

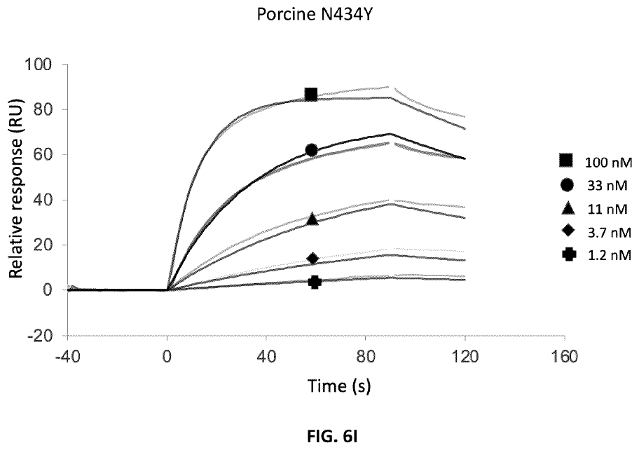

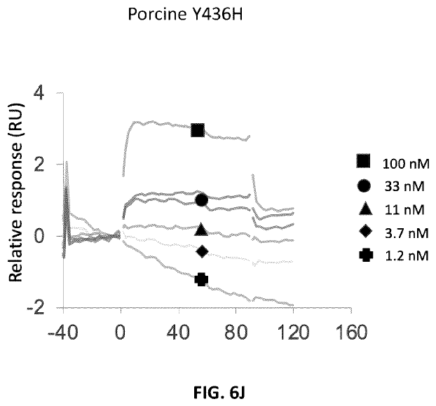

The kinetic binding data for the porcine IgG variants and wild-type are shown in Table 8 and the sensorgrams for the binding data are shown in FIGS. 6A-6J.

TABLE 8
Porcine IgG Fc variants and FcRn binding kinetics

| | | | | | Fold increase over |
|---|---|---|---|---|---|
| Porcine Variant | ka | kd | KD | Method for fitting data | wild-type KD |
| Porcine wild-type | | | 3.17E−07 | Steady state affinity | 1-fold |
| Porcine M252Y | 3.07E+05 | 2.99E−02 | 9.72E−08 | 1:1 kinetic interaction model | 3.3-fold |
| Porcine T286E | 4.77E+05 | 4.58E−02 | 9.61E−08 | 1:1 kinetic interaction model | 3.3-fold |
| Porcine Q309D | 2.64E+05 | 2.47E−02 | 9.36E−08 | 1:1 kinetic interaction model | 3.4-fold |
| Porcine Q309V | 2.57E+05 | 8.78E−03 | 3.41E−08 | 1:1 kinetic interaction model | 9.3-fold |
| Porcine Q311V | 2.81E+05 | 2.15E−02 | 7.64E−08 | 1:1 kinetic interaction model | 4.1-fold |
| Porcine A426Y | 3.74E+05 | 3.93E−02 | 1.05E−07 | 1:1 kinetic interaction model | 3.0-fold |
| Porcine M428Y | 3.00E+05 | 2.61E−02 | 8.69E−08 | 1:1 kinetic interaction model | 3.6-fold |
| Porcine N434Y | 7.35E+05 | 5.83E−03 | 7.93E−09 | 1:1 kinetic interaction model | 40-fold |
| Porcine Y436H | | | No binding | | |

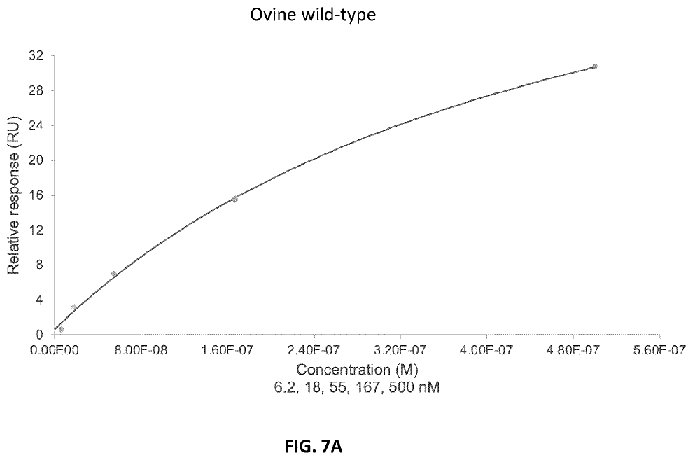

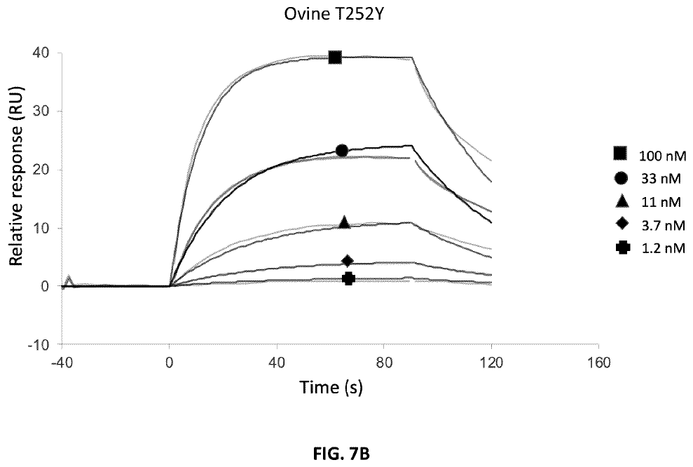

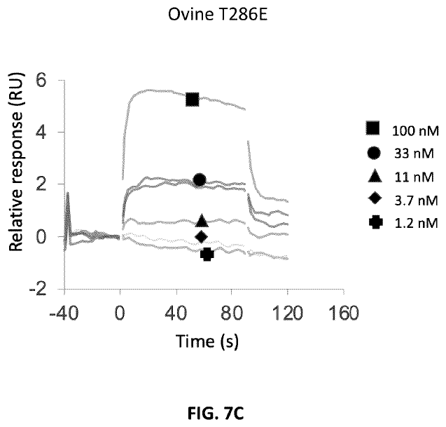

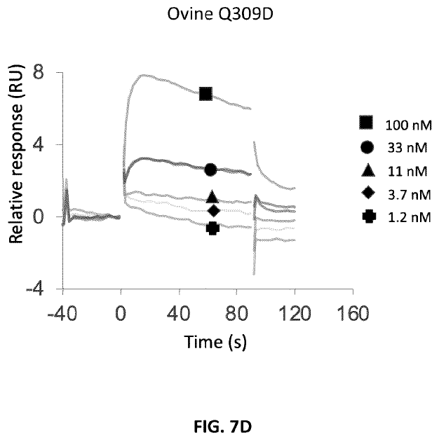

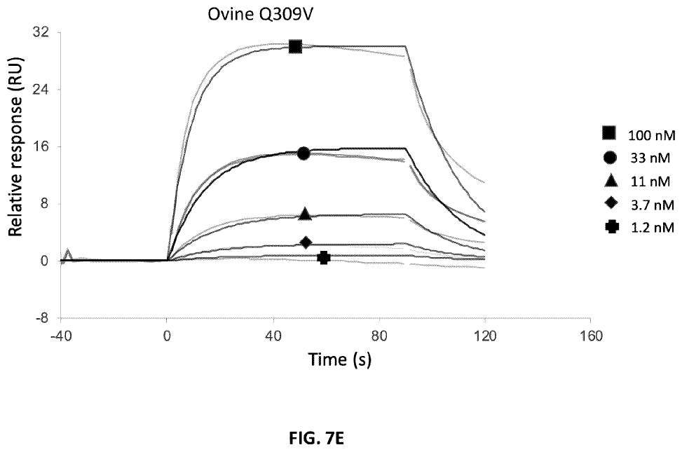

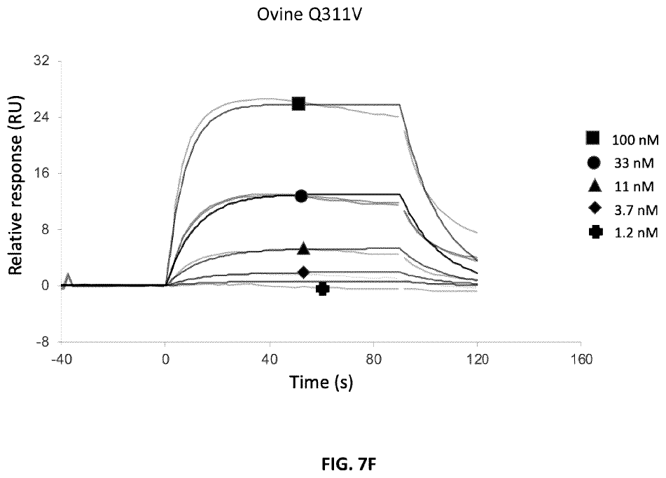

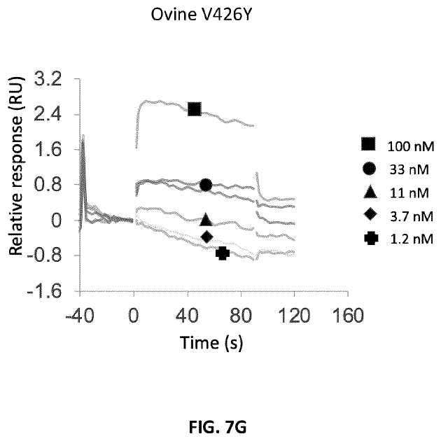

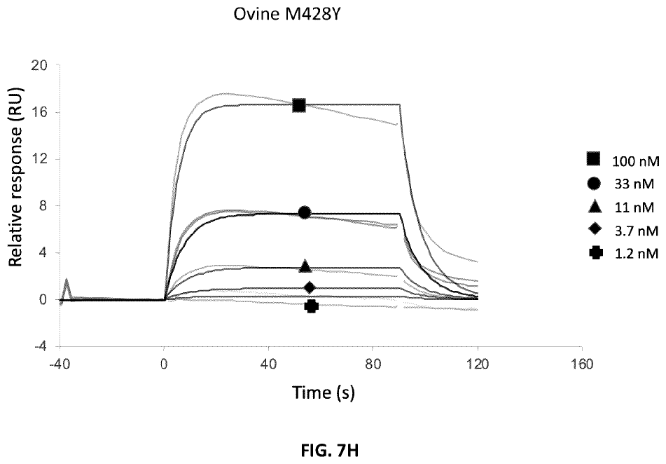

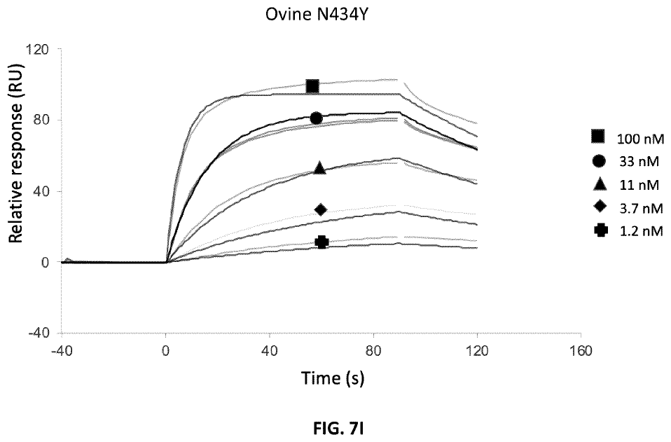

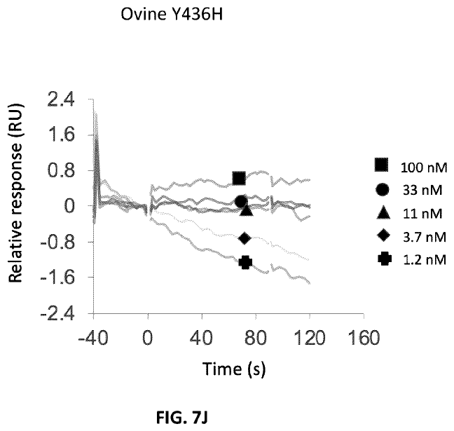

The kinetic binding data for the ovine IgG variants and wild-type are shown in Table 9 and the sensorgrams for the binding data are shown in FIGS. 7A-7J.

TABLE 9
Ovine IgG Fc variants and FcRn binding kinetics

| | | | | | Fold increase over |
|---|---|---|---|---|---|
| Ovine Variant | ka | kd | KD | Method for fitting data | wild-type KD |
| Ovine wild-type | | | 4.97E−07 | Steady state affinity | 1-fold |
| Ovine T252Y | 6.12E+05 | 2.64E−02 | 4.31E−08 | 1:1 kinetic interaction model | 11-fold |
| Ovine T286E | | | No binding | | |
| Ovine Q309D | | | No binding | | |
| Ovine Q309V | 6.28E+05 | 4.98E−02 | 7.93E−08 | 1:1 kinetic interaction model | 6.3-fold |
| Ovine Q311V | 7.08E+05 | 6.57E−02 | 9.27E−08 | 1:1 kinetic interaction model | 5.4-fold |
| Ovine V426Y | | | No binding | | |
| Ovine M428Y | 1.58E+06 | 2.63E−01 | 1.66E−07 | 1:1 kinetic interaction model | 3.0-fold |
| Ovine N434Y | 1.58E+06 | 9.60E−03 | 6.09E−09 | 1:1 kinetic interaction model | 81-fold |
| Ovine Y436H | | | No binding | | |

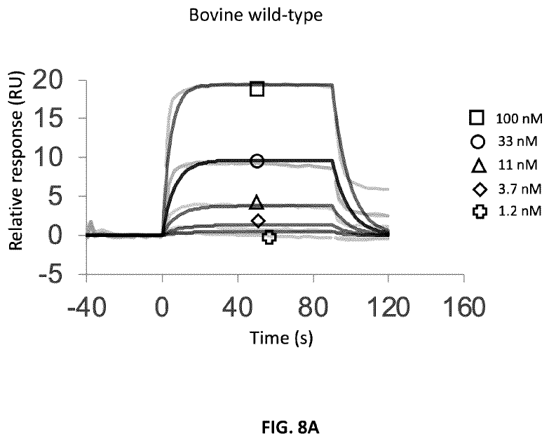

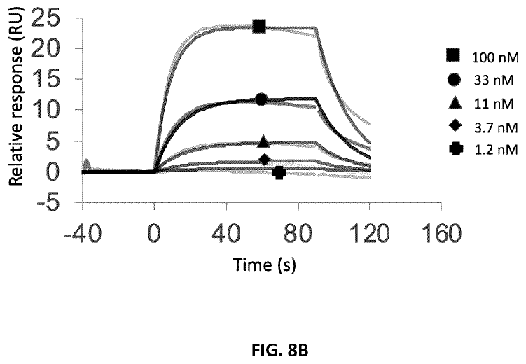

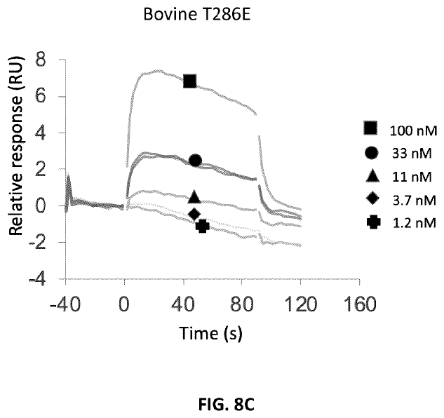

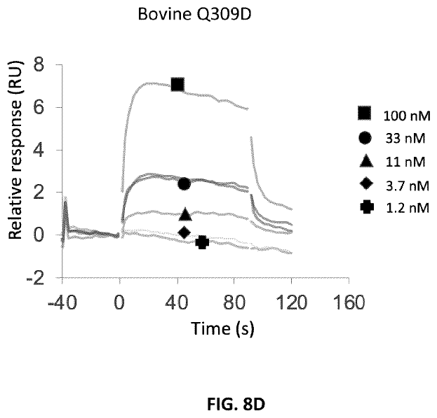

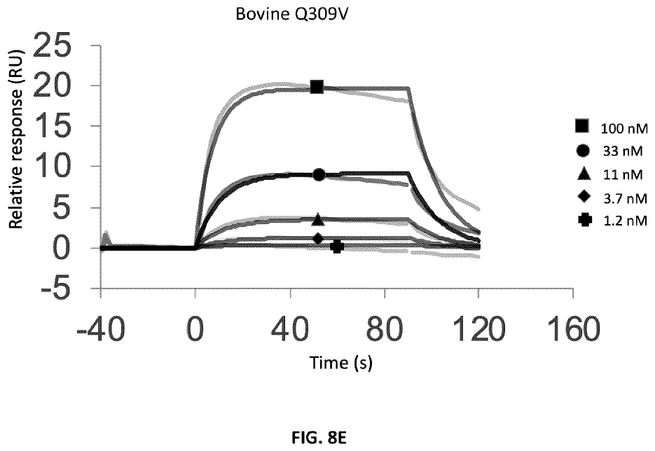

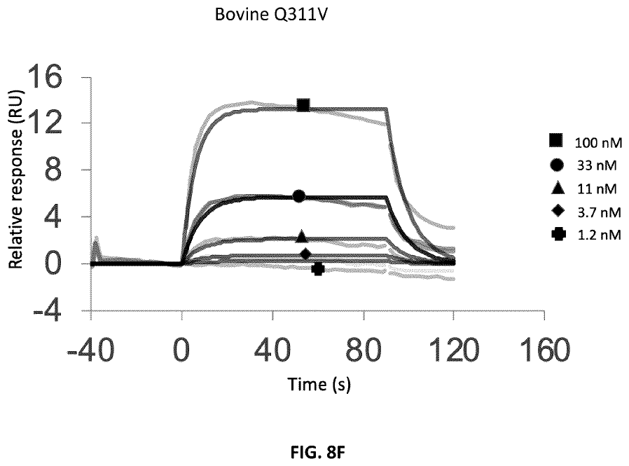

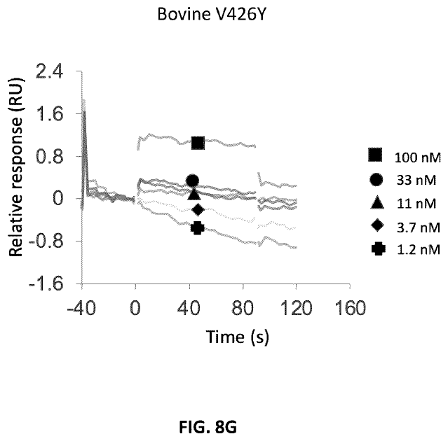

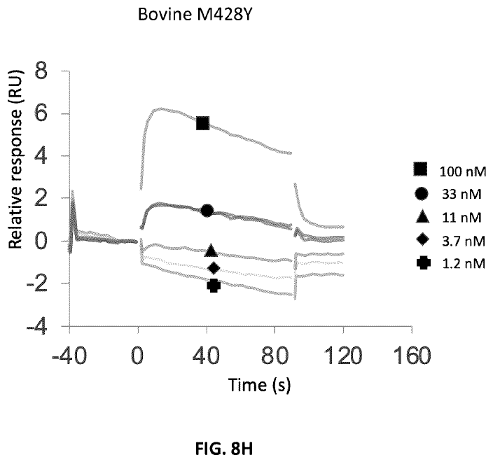

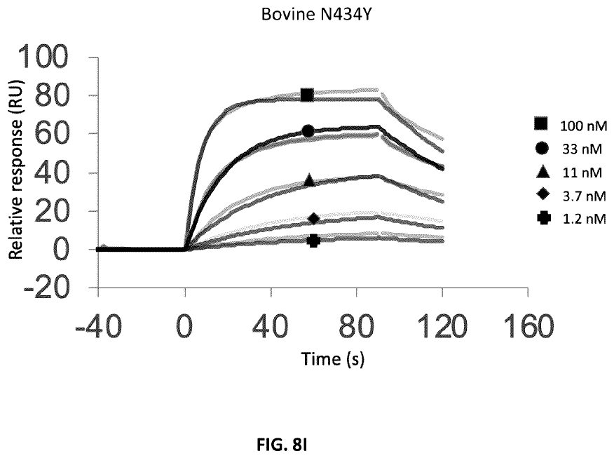

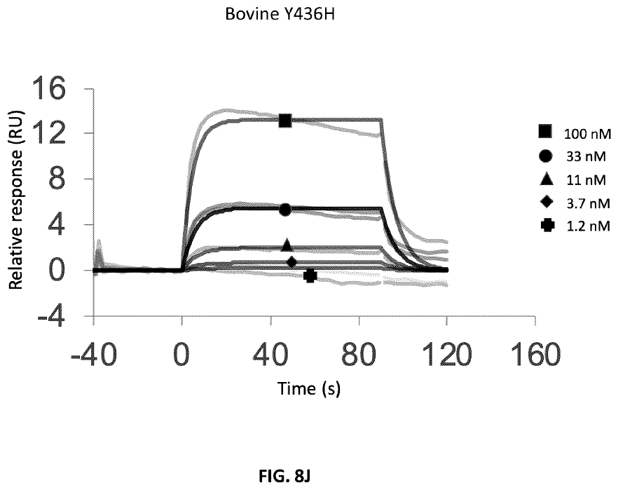

The kinetic binding data for the bovine IgG variants and wild-type are shown in Table 10 and the sensorgrams for the binding data are shown in FIGS. 8A-8J.

TABLE 10
Bovine IgG Fc variants and FcRn binding kinetics

| | | | | | Fold increase over |
|---|---|---|---|---|---|
| Bovine Variant | ka | kd | KD | Method for fitting data | wild-type KD |
| Bovine wild-type | 2.25E+05 | 1.14E−01 | 5.05E−07 | 1:1 kinetic interaction model | 1-fold |
| Bovine T252Y | 2.33E+05 | 1.66E−02 | 7.14E−08 | 1:1 kinetic interaction model | 7.1-fold |
| Bovine T286E | | | No binding | | |
| Bovine Q309D | | | No binding | | |
| Bovine Q309V | 5.94E+05 | 7.68E−02 | 1.29E−07 | 1:1 kinetic interaction model | 3.9-fold |
| Bovine Q311V | 5.97E+05 | 1.09E−07 | 1.82E−07 | 1:1 kinetic interaction model | 2.8-fold |
| Bovine V426Y | | | No binding | | |
| Bovine M428Y | | | No binding | | |
| Bovine N434Y | 1.19E+06 | 1.41E−02 | 1.19E−08 | 1:1 kinetic interaction model | 42-fold |
| Bovine Y436H | 6.77E+05 | 1.52E−01 | 2.24E−07 | 1:1 kinetic interaction model | 2.3-fold |

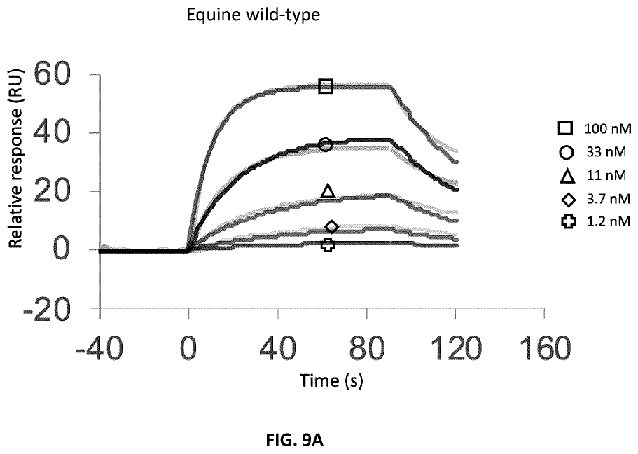

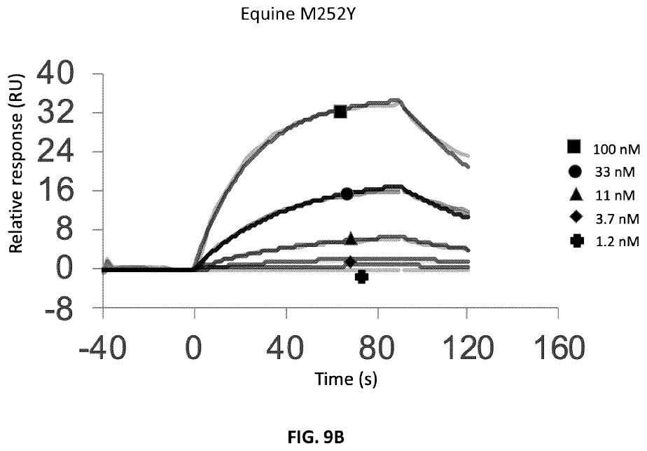

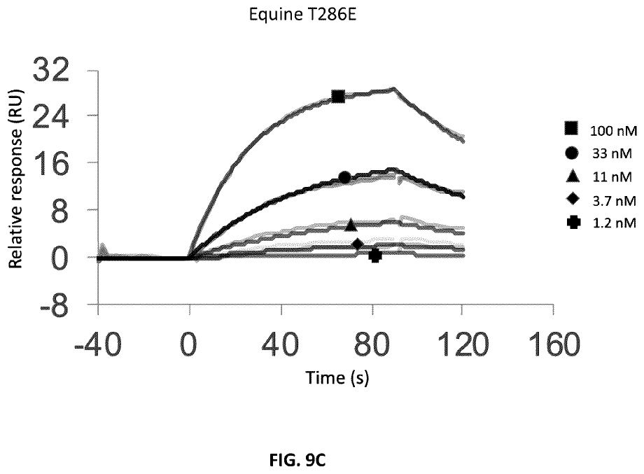

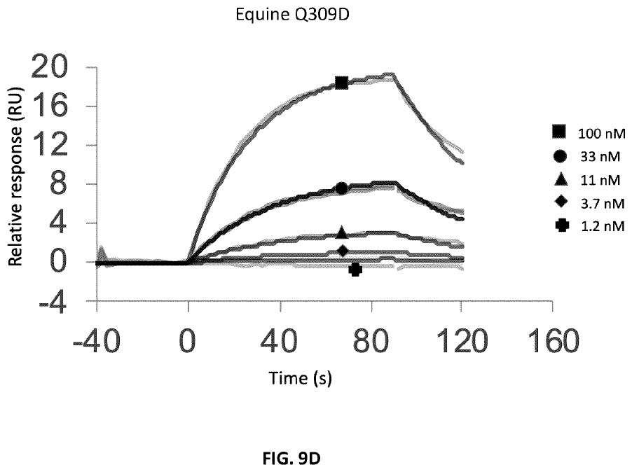

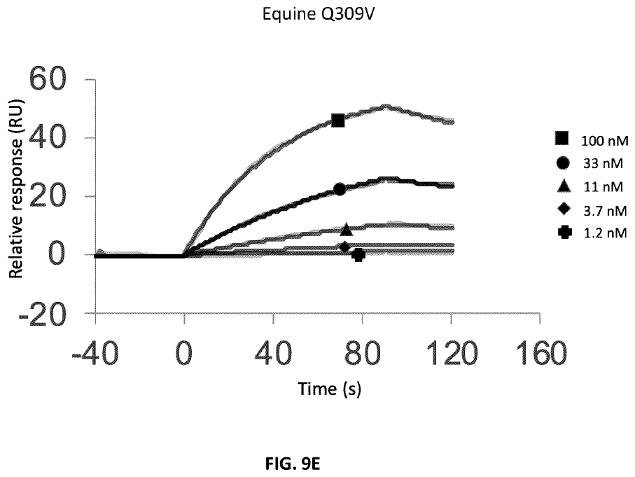

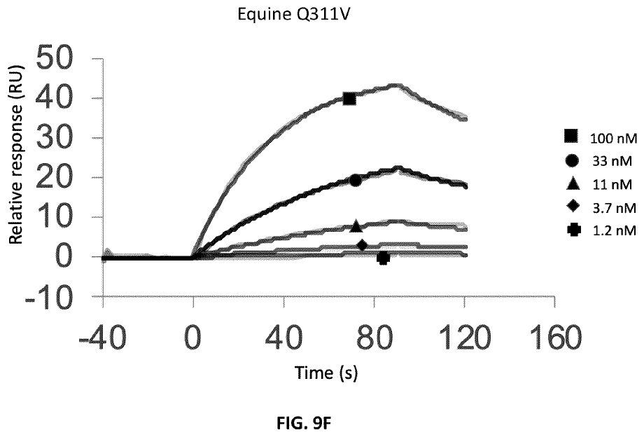

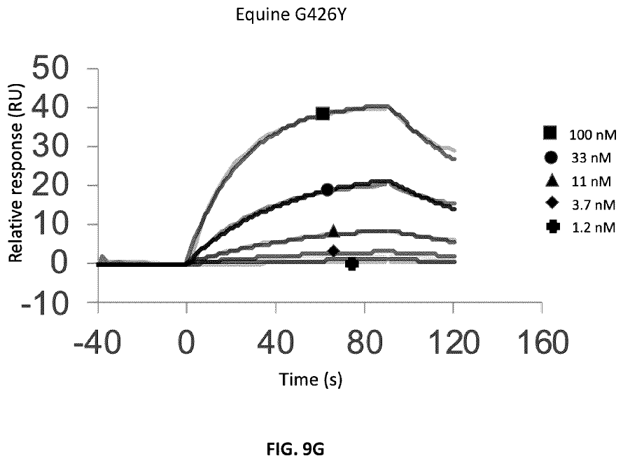

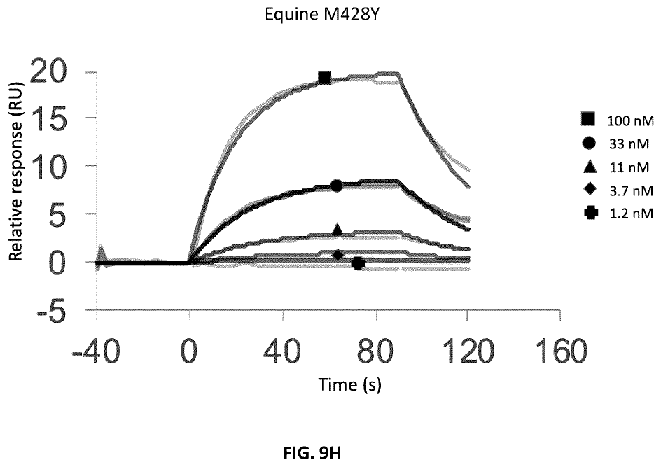

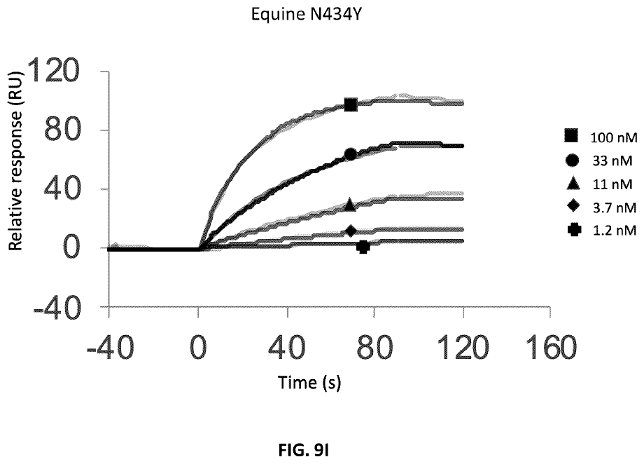

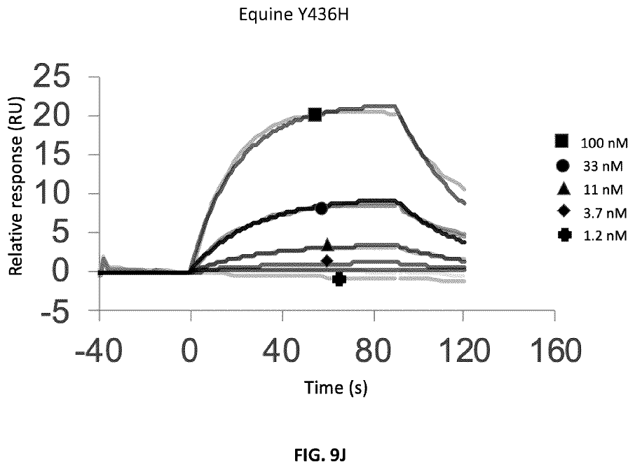

The kinetic binding data for the equine IgG variants and wild-type are shown in Table 11 and the sensorgrams for the binding data are shown in FIGS. 9A-9J.

TABLE 11
Equine IgG Fc variants and FcRn binding kinetics

| | | | | | Fold increase over |
|---|---|---|---|---|---|
| Equine Variant | ka | kd | KD | Method for fitting data | wild-type KD |
| Equine wild-type | 1.47E−05 | 2.10E−02 | 1.43E−07 | 1:1 kinetic interaction model | |
| Equine M252Y | 2.33E+05 | 1.66E−02 | 7.14E−08 | 1:1 kinetic interaction model | 2.0-fold |
| Equine T286E | 2.55E+05 | 1.22E−02 | 4.80E−08 | 1:1 kinetic interaction model | 3.0-fold |
| Equine Q309D | 1.51E+05 | 2.17E−02 | 1.44E−07 | 1:1 kinetic interaction model | 1.0-fold |
| Equine Q309V | 1.98E+05 | 4.00E−03 | 2.02E−08 | 1:1 kinetic interaction model | 7.2-fold |
| Equine Q311V | 2.16E+05 | 7.71E−03 | 3.57E−08 | 1:1 kinetic interaction model | 4.0-fold |
| Equine G426Y | 2.69E+05 | 1.41E−02 | 5.23E−08 | 1:1 kinetic interaction model | 2.8-fold |
| Equine M428Y | 2.01E+05 | 3.03E−02 | 1.51E−07 | 1:1 kinetic interaction model | 0.9-fold |
| Equine N434Y | 4.16E+05 | 9.78E−04 | 2.35E−09 | 1:1 kinetic interaction model | 61-fold |
| Equine Y436H | 1.91E+05 | 2.98E−02 | 1.56E−07 | 1:1 kinetic interaction model | 0.9-fold |

Table 12 shows a species comparison of the fold increase of each Fc variant compared to the wild-type Fc.

TABLE 12
Comparison of IgG Fc variants and FcRn binding among different species

| Species | 252Y | 286E | 309D | 309V | 311V | 426Y | 428Y | 434Y | 436H |
|---|---|---|---|---|---|---|---|---|---|
| Porcine | 3.3-fold | 3.3-fold | 3.4-fold | 9.3-fold | 4.1-fold | 3.0-fold | 3.6-fold | 40-fold | No binding |
| Ovine | 11-fold | No binding | No binding | 6.3-fold | 5.4-fold | No binding | 3.0-fold | 81-fold | No binding |
| Bovine | 7.1-fold | No binding | No binding | 3.9-fold | 2.8-fold | No binding | No binding | 42-fold | 2.3-fold |
| Equine | 2.0-fold | 3.0-fold | 1.0-fold | 7.2-fold | 4.0-fold | 2.8-fold | 0.9-fold | 61-fold | 0.9-fold |

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A polypeptide comprising an equine IgG Fc region variant, or an equine FcRn-binding region thereof, wherein the polypeptide comprises Val at a position that corresponds to amino acid position 309 of a wild type equine IgG, wherein the amino acid position is based on EU numbering, and wherein the polypeptide has increased binding affinity to equine FcRn when compared to a control polypeptide, wherein the control polypeptide is identical to the polypeptide except for having the wild type equine IgG Fc region or a FcRn-binding region thereof in place of the equine IgG Fc region variant or the equine FcRn-binding region thereof.

2. The polypeptide of claim 1, further comprising Glu at a position that corresponds to amino acid position 286 of the wild type equine IgG Fc region.

3. The polypeptide of claim 1, wherein the wild type equine IgG is selected from the group consisting of an equine IgG1, IgG2, IgG3, IgG4, IgG5, IgG6, and IgG7.

4. The polypeptide of claim 1, wherein the wild type equine IgG Fc is:
(a) an equine IgG1 Fc comprising the amino acid sequence of SEQ ID NO: 15;
(b) an equine IgG2 Fc comprising the amino acid sequence of SEQ ID NO: 16;
(c) an equine IgG3 Fc comprising the amino acid sequence of SEQ ID NO: 17;
(d) an equine IgG4 Fc comprising the amino acid sequence of SEQ ID NO: 18;
(e) an equine IgG5 Fc comprising the amino acid sequence of SEQ ID NO: 19;
(f) an equine IgG6 Fc comprising the amino acid sequence of SEQ ID NO: 20; or
(g) an equine IgG1 Fc comprising the amino acid sequence of SEQ ID NO: 21.

5. The polypeptide of claim 1, wherein the polypeptide further comprises a binding domain.

6. The polypeptide of claim 5, wherein the binding domain comprises (i) six complementarity determining regions (CDRs) of an immunoglobulin molecule; (ii) a ligand binding domain of an equine receptor protein, (iii) a nanobody, or (iv) an extracellular domain of an equine receptor protein.

7. The polypeptide of claim 5, wherein the binding domain specifically binds to an antigen selected from the group consisting of NGF, TrkA, ADAMTS, IL-1, IL-2, IL-4, IL-4R, Angiotensin type 1 (AT1) receptor, Angiotensin type 2 (AT2) receptor, IL-5, IL-12, IL-13, IL-31, IL-33, CD3, CD20, CD47, CD52, and complement system complex.

8. The polypeptide of claim 1, further comprising a protein selected from the group consisting of EPO, CTLA4, LFA3, VEGFR1, VEGFR3, IL-1R, IL-4R, GLP-1 receptor agonist, and Thrombopoietin binding peptide.

9. The polypeptide of claim 1, wherein the polypeptide binds to an equine FcRn at a higher level at an acidic pH than at a neutral pH.

10. The polypeptide of claim 9, wherein the polypeptide binds to an equine FcRn at a higher level at pH 5.5 than at pH 7.4.

11. The polypeptide of claim 9, wherein the polypeptide binds to an equine FcRn at a higher level at pH 6.0 than at pH 7.4.

12. A pharmaceutical composition comprising (i) the polypeptide of claim 1 and (ii) a pharmaceutically acceptable excipient.

13. A nucleic acid or nucleic acids encoding the polypeptide of claim 1.

14. An expression vector or expression vectors comprising the nucleic acid or nucleic acids of claim 13.

15. A host cell comprising the expression vector or expression vectors of claim 14.

16. A method of making a polypeptide, the method comprising:
(a) providing the nucleic acid or nucleic acids of claim 13;
(b) expressing the nucleic acid or nucleic acids in a host cell culture, thereby producing the polypeptide; and
(c) collecting the polypeptide produced in (b) from the host cell culture.

17. The method of claim 16, further comprising formulating the polypeptide as a pharmaceutical formulation.

18. A polypeptide comprising an equine IgG Fc region variant, or an equine FcRn-binding region thereof, wherein the polypeptide comprises Val at a position that corresponds to amino acid position 309 and Glu at a position that corresponds to amino acid position 286 of a wild type equine IgG, wherein the amino acid positions are based on EU numbering, and wherein the polypeptide has increased binding affinity to equine FcRn when compared to a control polypeptide, wherein the control polypeptide is identical to the polypeptide except for having the wild type equine IgG Fc region or a FcRn-binding region thereof in place of the equine IgG Fc region variant or the equine FcRn-binding region thereof.


SEQUENCE LISTING (36 sequences)

SEQ ID NO: 1 - protein, 218 aa, Sus scrofa
GCEVAGPSVFIFPPKPKDTLMISQTPEVTCVVVDVSKEHAEVQFSWYVDGVEVHTAETRPKEEQFNSTYRVVSVLPIQHQDWLKGKEFKCKVNNVDLPAPITRTISKAIGQSREPQVYTLPPPAEELSRSKVTVTCLVIGFYPPDIHVEWKSNGQPEPEGNYRTTPPQQDVDGTFFLYSKLAVDKARWDHGETFECAVMHEALHNHYTQKSISKTQGK

SEQ ID NO: 2 - protein, 218 aa, Sus scrofa
GCEVAGPSVFIFPPKPKDTLMISQTPEVTCVVVDVSKEHAEVQFSWYVDGVEVHTAETRPKEEQFNSTYRVVSVLPIQHQDWLKGKEFKCKVNNVDLPAPITRTISKAIGQSREPQVYTLPPPAEELSRSKVTLTCLVIGFYPPDIHVEWKSNGQPEPENTYRTTPPQQDVDGTFFLYSKLAVDKARWDHGDKFECAVMHEALHNHYTQKSISKTQGK

SEQ ID NO: 3 - protein, 218 aa, Sus scrofa
ACESPGPSVFIFPPKPKDTLMISRTPQVTCVVVDVSQENPEVQFSWYVDGVEVHTAQTRPKEEQFNSTYRVVSVLPIQHQDWLNGKEFKCKVNNKDLPAPITRIISKAKGQTREPQVYTLPPHAEELSRSKVSITCLVIGFYPPDIDVEWQRNGQPEPEGNYRTTPPQQDVDGTYFLYSKFSVDKASWQGGGIFQCAVMHEALHNHYTQKSISKTPGK

SEQ ID NO: 4 - protein, 218 aa, Sus scrofa; identical to SEQ ID NO: 3

SEQ ID NO: 5 - protein, 219 aa, Sus scrofa
AAEVLGAPSVFLFPPKPKDILMISRTPKVTCVVVDVSQEEAEVQFSWYVDGVQLYTAQTRPMEEQFNSTYRVVSVLPIQHQDWLKGKEFKCKVNNKDLLSPITRTISKATGPSRVPQVYTLPPAWEELSKSKVSITCLVTGFYPPDIDVEWQSNGQQEPEGNYRTTPPQQDVDGTYFLYSKLAVDKVRWQRGDLFQCAVMHEALHNHYTQKSISKTQGK

SEQ ID NO: 6 - protein, 218 aa, Sus scrofa
ACEGPGPSAFIFPPKPKDTLMISRTPKVTCVVVDVSQENPEVQFSWYVDGVEVHTAQTRPKEEQFNSTYRVVSVLPIQHQDWLNGKEFKCKVNNKDLPAPITRIISKAKGQTREPQVYTLPPPTEELSRSKVTLTCLVTGFYPPDIDVEWQRNGQPEPEGNYRTTPPQQDVDGTYFLYSKLAVDKASWQRGDTFQCAVMHEALHNHYTQKSIFKTPGK

SEQ ID NO: 7 - protein, 208 aa, Sus scrofa
ACEGPGPSAFIFPPKPKDTLMISRTPKVTCVVVDVSQENPEVQFSWYVDGVEVHTAQTRPKEEQFNSTYRVVSVLLIQHQDWLNGKEFKCKVNNKDLPAPITRIISKAKGQTREPQVYTLPPPTEELSRSKVTLTCLVTGFYPPDIDVEWQRNGQPEPEGNYRTTPPQQDVDGTYFLYSKLAVDKASWQRGDTFQCAVMHEALHNHYT

SEQ ID NO: 8 - protein, 218 aa, Sus scrofa
GCEVAGPSVFIFPPKPKDILMISRTPEVTCVVVDVSKEHAEVQFSWYVDGEEVHTAETRPKEEQFNSTYRVVSVLPIQHEDWLKGKEFECKVNNEDLPGPITRTISKAKGVVRSPEVYTLPPPAEELSKSIVTLTCLVKSIFPPFIHVEWKINGKPEPENAYRTTPPQEDEDRTYFLYSKLAVDKARWDHGETFECAVMHEALHNHYTQKSISKTQGK

SEQ ID NO: 9 - protein, 217 aa, Sus scrofa
ICPVAGPSVFIFPPKPKDILMISRTPEVTCVVVDVSKEHAEVQFSWYVDGEEVHTAETRPKEEQFNSTYRVVSVLPIQHEDWLKGKEFECKVNNEDLPGPITRTISKAKGVVRSPEVYTLPPPAEELSKSIVTLTCLVKSFFPPFIHVEWKINGKPEPENAYRTTPPQEDEDGTYFLYSKFSVEKFRWHSGGIHCAVMHEALHNHYTEKSVSQTPGK

SEQ ID NO: 10 - protein, 218 aa, Sus scrofa
ACEGPGPSAFIFPPKPKDTLMISRTPKVTCVVVDVSQENPEVQFSWYVDGVEVHTAQTRPKEEQFNSTYRVVSVLPIQHQDWLNGKEFKCKVNNKDLPAPITRIISKAKGQTREPQVYTLPPPTEELSRSKLSVTCLITGFYPPDIDVEWQRNGQPEPEGNYRTTPPQQDVDGTYFLYSKLAVDKASWQRGDPFQCAVMHEALHNHYTQKSIFKTPGN

SEQ ID NO: 11 - protein, 214 aa, Sus scrofa
ACEGNGPSVFIFPPKPKDTLMISRTPEVTCVVVDVSQENPEVQFSWYVDGEEVHTAETRPKEEQFNSTYRVVSVLPIQHQDWLKGKEFECKVNNKDLPAPITRIISKAKGPSREPQVYTLSPSAEELSRSKVSITCLVTGFYPPDIDVEWKSNGQPEPEGNYRSTPPQEDEDGTYFLYSKLAVDKARLQSGGIHCAVMHEALHNHYTQKSISKT

SEQ ID NO: 12 - protein, 217 aa, Bos taurus
PPELPGGPSVFIFPPKPKDTLTISGTPEVTCVVVDVGHDDPEVKFSWFVDNVEVNTATTKPREEQFNSTYRVVSALRIQHQDWTGGKEFKCKVHNEGLPAPIVRTISRTKGPAREPQVYVLAPPQEELSKSTVSLTCMVTSFYPDYIAVEWQRNGQPESEDKYGTTPPQLDADSSYFLYSKLRVDRNSWQEGDTYTCVVMHEALHNHYTQKSTSKSA

SEQ ID NO: 13 - protein, 216 aa, Bos taurus
CVREPSVFIFPPKPKDTLMITGTPEVTCVVVNVGHDNPEVQFSWFVDDVEVHTARTKPREEQFNSTYRVVSALPIQHQDWTGGKEFKCKVNIKGLSASIVRIISRSKGPAREPQVYVLDPPKEELSKSTVSVTCMVIGFYPEDVDVEWQRDRQTESEDKYRTTPPQLDADRSYFLYSKLRVDRNSWQRGGTYTCVVMHEALHNHYMQKSTSKSAGK

SEQ ID NO: 14 - protein, 217 aa, Bos taurus
EPLGGLSVFIFPPKPKDTLTISGTPEVTCVVVDVGQDDPEVQFSWFVDDVEVHTARTKPREEQFNSTYRVVSALRIQHQDWLQGKEFKCKVNNKGLPAPIVRTISRTKGQAREPQVYVLAPPREELSKSTLSLTCLITGFYPEEIDVEWQRNGQPESEDKYHTTAPQLDADGSYFLYSKLRVNKSSWQEGDHYTCAVMHEALRNHYKEKSISRSPGK

SEQ ID NO: 15 - protein, 219 aa, Equus caballus
APELLGGPSVFIFPPNPKDTLMITRTPEVTCVVVDVSQENPDVKFNWYMDGVEVRTATTRPKEEQFNSTYRVVSVLRIQHQDWLSGKEFKCKVNNQALPQPIERTITKTKGRSQEPQVYVLAPHPDELSKSKVSVTCLVKDFYPPEINIEWQSNGQPELETKYSTTQAQQDSDGSYFLYSKLSVDRNRWQQGTTFTCGVMHEALHNHYTQKNVSKNPGK

SEQ ID NO: 16 - protein, 213 aa, Equus caballus
GPSVFIFPPNPKDALMISRTPVVTCVVVNLSDQYPDVQFSWYVDNTEVHSAITKQREAQFNSTYRVVSVLPIQHQDWLSGKEFKCSVTNVGVPQPISRAISRGKGPSRVPQVYVLPPHPDELAKSKVSVTCLVKDFYPPDISVEWQSNRWPELEGKYSTTPAQLDGDGSYFLYSKLSLETSRWQQVESFTCAVMHEALHNHFTKTDISESLGK

SEQ ID NO: 17 - protein, 219 aa, Equus caballus
APELLGGPSVFIFPPKPKDVLMITRMPEVTCLVVDVSHDSSDVLFTWYVDGTEVKTAKTMPNEEQNNSTYRVVSVLRIQHQDWLNGKKFKCKVNNQALPAPVERTISKATGQTRVPQVYVLAPHPDELSKNKVSVTCLVKDFYPTDITVEWQSNEHPEPEGKYRTTEAQKDSDGSYFLYSKLTVEKDRWQQGTTFTCVVMHEALHNHVMQKNISKNPGK

SEQ ID NO: 18 - protein, 218 aa, Equus caballus
ECLQVGPSVFIFPPKPKDVLMISRTPTVTCVVVDVGHDFPDVQFNWYVDGVETHTATTEPKQEQFNSTYRVVSVLPIQHKDWLSGKEFKCKVNNKALPAPVERTISKPTGQPREPQVYVLAPHRDELSKNKVSVTCLVKDFYPTDIDIEWKSNGQPEPETKYSTTPAQLDSDGSYFLYSKLTVETNRWQQGTTFTCAVMHEALHNHYTEKSVSKSPGK

SEQ ID NO: 19 - protein, 219 aa, Equus caballus
APELPGGPSVFIFPPKPKDVLKISRKPEVTCVVVDLGHDDPDVQFTWFVDGVETHTATTEPKEEQFNSTYRVVSVLPIQHQDWLSGKEFKCSVTNKALPAPVERTTSKAKGQLRVPQVYVLAPHPDELAKNTVSVTCLVKDFYPPEIDVEWQSNEHPEPEGKYSTTPAQLNSDGSYFLYSKLSVETSRWKQGESFTCGVMHEAVENHYTQKNVSHSPGK

SEQ ID NO: 20 - protein, 219 aa, Equus caballus
DSKFLGRPSVFIFPPNPKDTLMISRTPEVTCVVVDVSQENPDVKFNWYVDGVEAHTATTKAKEKQDNSTYRVVSVLPIQHQDWRRGKEFKCKVNNRALPAPVERTITKAKGELQDPKVYILAPHREEVTKNTVSVTCLVKDFYPPDINVEWQSNEEPEPEVKYSTTPAQLDGDGSYFLYSKLTVETDRWEQGESFTCVVMHEAIRHTYRQKSITNFPGK

SEQ ID NO: 21 - protein, 218 aa, Equus caballus
ECLSVGPSVFIFPPKPKDVLMISRTPTVTCVVVDVGHDFPDVQFNWYVDGVETHTATTEPKQEQNNSTYRVVSILAIQHKDWLSGKEFKCKVNNQALPAPVQKTISKPTGQPREPQVYVLAPHRDELSKNKVSVTCLVKDFYPTDIDIEWKSNGQPEPETKYSTTPAQLDSDGSYFLYSKLTVETNRWQQGTTFTCAVMHEALHNHYTEKSVSKSPGK

SEQ ID NO: 22 - protein, 218 aa, Ovis aries
PELPGGPSVFIFPPKPKDTLTISGTPEVTCVVVDVGQDDPEVQFSWFVDNVEVRTARTKPREEQFNSTFRVVSALPIQHQDWTGGKEFKCKVHNEALPAPIVRTISRTKGQAREPQVYVLAPPQEELSKSTLSVTCLVTGFYPDYIAVEWQKNGQPESEDKYGTTTSQLDADGSYFLYSRLRVDKNSWQEGDTYACVVMHEALHNHYTQKSISKPPGK

SEQ ID NO: 23 - protein, 215 aa, Ovis aries
VSRPSVFIFPPKPKDSLMITGTPEVTCVVVDVGQGDPEVQFSWFVDNVEVRTARTKPREEQFNSTFRVVSALPIQHDHWTGGKEFKCKVHSKGLPAPIVRTISRAKGQAREPQVYVLAPPQEELSKSTLSVTCLVTGFYPDYIAVEWQRARQPESEDKYGTTTSQLDADGSYFLYSRLRVDKSSWQRGDTYACVVMHEALHNHYTQKSISKPPGK

SEQ ID NO: 24 - protein, 218 aa, Ovis aries
PEPLGGLSVFIFPPKPKDTLTISGTPEVTCVVVDVGQDDPEVQFSWFVDNVEVRTARTKPREEQFNSTFRVVSALPIQHQDWLRGKEIKCKVHNKGLPAPIVRTISRTKGQAREPQVYVLAPPQEELSKSTLSVTCLVTGFYPDYIAVEWQKNGQPESEDKYGTTTSQLDADGSYFLYSRLRVDKNSWQEGDTYACVVMHEALHNHYTQKSISKPPGK

SEQ ID NO: 25 - protein, 218 aa, Capra hircus
PELPGGPSVFIFPPKPKDTLTISGKPEVTCVVVDVGQDDPEVQFSWFVDNVEVHTARTKPREEQFNSTFRVVSALPIQHQDWTGGKEFKCKVHNEGLPAPIVRTISRTKGQAREPQVYVLAPPQEELSKSTLSVTCLVTGFYPDYIAVEWQRNGQPESEDKYGTTTSQLDADGSYFLYSRLRVNKSSWQEGDTYACVVMHEALHNHYTQKSISKPPGK

SEQ ID NO: 26 - protein, 215 aa, Capra hircus
VRGPSVFIFPPKPKDSLMITGTPEVTCVVVDVGQDDPEVQFSWFVDNVEVHTARTKPREEQFNSTFRVVSALPIQHDHWTGGKEFKCKVNNKALPAPIVRTISRDKGQAREPQVYVLAPPQEELSKSTLSVTCLVTGFYPDYIAVEWQRARQPESEDKYGTTTSQLDADGSYFLYSRLRVDKSSWQEGDTYACVVMHEALHNHYTQKSISKPPGK

SEQ ID NO: 27 - protein, 218 aa, Capra hircus
PEPLGGLSVFIFPPKPKDTLTISGTPEVTCVVVDVGQDDPEVQFSWFMDNVEVHTARTTPREEQFNSTFRVVSALPIQHKDWLQGKEFKCKVHNEGLPAPIIRTISRAKGQAREPQVYVLAPPREELSKSTLSVTCLITGFYPEEVDVEWQRDGQPESEDKYHTAPPQLDADGSYFLYSRLRVNKSSWQEGDTYTCAVMHEALRNHYKEKSISKSPGK

SEQ ID NO: 28 - protein, 15 aa, synthetic construct
GLNDIFEAQKIEWHE

SEQ ID NO: 29 - protein, 8 aa, synthetic construct
DYKDDDDK

SEQ ID NO: 30 - protein, 202 aa, Sus scrofa
VFIFPPKPKDTLMISQTPEVTCVVVDVSKEHAEVQFSWYVDGVEVHTAETRPKEEQFNSTYRVVSVLPIQHQDWLKGKEFKCKVNNVDLPAPITRTISKAIGQSREPQVYTLPPPAEELSRSKVTVTCLVIGFYPPDIHVEWKSNGQPEPEGNYRTTPPQQDVDGTFFLYSKLAVDKARWDHGETFECAVMHEALHNHYTQK

SEQ ID NO: 31 - protein, 202 aa, Ovis aries
VFIFPPKPKDTLTISGTPEVTCVVVDVGQDDPEVQFSWFVDNVEVRTARTKPREEQFNSTFRVVSALPIQHQDWTGGKEFKCKVHNEALPAPIVRTISRTKGQAREPQVYVLAPPQEELSKSTLSVTCLVTGFYPDYIAVEWQKNGQPESEDKYGTTTSQLDADGSYFLYSRLRVDKNSWQEGDTYACVVMHEALHNHYTQK

SEQ ID NO: 32 - protein, 202 aa, Bos taurus
VFIFPPKPKDTLTISGTPEVTCVVVDVGHDDPEVKFSWFVDNVEVNTATTKPREEQFNSTYRVVSALRIQHQDWTGGKEFKCKVHNEGLPAPIVRTISRTKGPAREPQVYVLAPPQEELSKSTVSLTCMVTSFYPDYIAVEWQRNGQPESEDKYGTTPPQLDADSSYFLYSKLRVDRNSWQEGDTYTCVVMHEALHNHYTQK

SEQ ID NO: 33 - protein, 202 aa, Equus caballus
VFIFPPNPKDTLMITRTPEVTCVVVDVSQENPDVKFNWYMDGVEVRTATTRPKEEQFNSTYRVVSVLRIQHQDWLSGKEFKCKVNNQALPQPIERTITKTKGRSQEPQVYVLAPHPDELSKSKVSVTCLVKDFYPPEINIEWQSNGQPELETKYSTTQAQQDSDGSYFLYSKLSVDRNRWQQGTTFTCGVMHEALHNHYTQK

SEQ ID NO: 34 - protein, 202 aa, Felis catus
IFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQK

SEQ ID NO: 35 - protein, 202 aa, Canis lupus
  source 1..202: note = subspecies familiaris
VFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQE

SEQ ID NO: 36 - protein, 199 aa, Homo sapiens
VFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQK